(19) United States
(12) Patent Application Publication
(10) Pub. No.: US 2023/0286950 A1
(43) Pub. Date: Sep. 14, 2023

(54) METHODS OF SYNTHESIZING DEUTERATED SUBSTITUTED PYRIDINONE-PYRIDINYL COMPOUNDS

(71) Applicant: ACLARIS THERAPEUTICS, INC., Wayne, PA (US)

(72) Inventors: Gary A. DECRESCENZO, Parkville, MO (US); John Robert SPRINGER, Wentzville, MO (US); Jon P. LAWSON, Wildwood, MO (US)

(21) Appl. No.: 18/164,286

(22) Filed: Feb. 3, 2023

Related U.S. Application Data
(60) Provisional application No. 63/306,692, filed on Feb. 4, 2022.
(60) Provisional application No. 63/344,834, filed on May 23, 2022.

(51) Int. Cl. C07D 401/14

(52) U.S. Cl. CPC C07D 401/14; C07B 2200/05; C07B 2200/13

(57) ABSTRACT

The present disclosure provides methods of synthesizing a compound of Formula (P)-I and a compound of Formula (P)-II. The method proceeds through several different key pathways including a novel chiral separation as well as through various halide containing intermediates. Also disclosed are single enantiomers of the compound of Formula (P)-II.

### SUMMARY

The present disclosure includes embodiments directed to methods of synthesizing a compound of Formula (P)-I and synthesizing a compound of Formula (P)-II, having the structures:

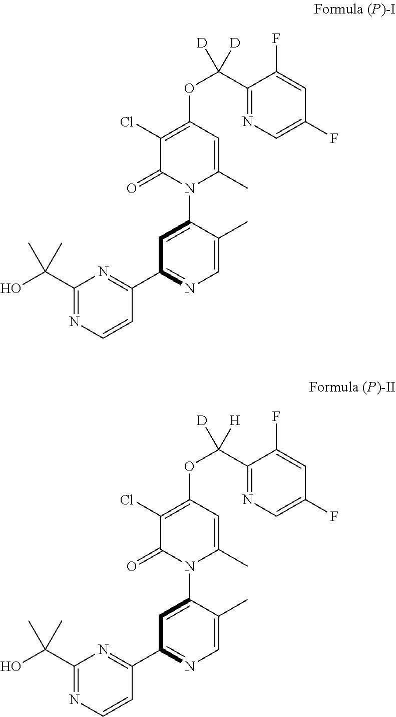

The methods include a chiral separation to produce the compound of Formula (P)-I or the compound of Formula (P)-II.

### Definitions

Before the present compositions and methods are described, it is to be understood that this invention is not limited to the particular processes, formulations, compositions, or methodologies described, as these may vary. It is also to be understood that the terminology used in the description is for the purpose of describing the particular versions or embodiments only, and is not intended to limit the scope of embodiments herein which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of embodiments herein, the preferred methods, devices, and materials are now described. All publications mentioned herein are incorporated by reference in their entirety. Nothing herein is to be construed as an admission that embodiments herein are not entitled to antedate such disclosure by virtue of prior invention.

It must also be noted that as used herein and in the appended claims, the singular forms “a,” “an,” and “the” include plural reference unless the context clearly dictates otherwise.

The transitional term “comprising,” which is synonymous with “including,” “containing,” or “characterized by,” is inclusive or open-ended and does not exclude additional, unrecited elements or method steps.

In embodiments or claims where the term “comprising” is used as the transition phrase, such embodiments can also be envisioned with replacement of the term “comprising” with the terms “consisting of” or “consisting essentially of.”

As used herein, the term “consists of” or “consisting of” means that the composition, formulation or the method includes only the elements, steps, or ingredients specifically recited in the particular claimed embodiment or claim.

As used herein, the term “consisting essentially of” or “consists essentially of” means that the composition, formulation or the method includes only the elements, steps or ingredients specifically recited in the particular claimed embodiment or claim and may optionally include additional elements, steps or ingredients that do not materially affect the basic and novel characteristics of the particular embodiment or claim. For example, the only active ingredient(s) in the formulation or method that treats the specified condition (e.g., nutrient depletion) is the specifically recited therapeutic(s) in the particular embodiment or claim.

As used herein, two embodiments are “mutually exclusive” when one is defined to be something which is different from the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean plus or minus 10% of the numerical value of the number with which it is being used. Therefore, about 50% means in the range of 45%-55%.

In embodiments or claims the “X” in the term “MeMgX” is a halogen.

The term “chiral separation,” as used herein, refers to the separation of racemic compounds into their single or enriched atropisomers or enantiomers.

The term “substantially free” as used herein, is used interchangeably with, the term “substantially pure”, refers to a compound which is free from all other compounds within the limits of detection as measured by any means including nuclear magnetic resonance (NMR), gas chromatography/mass spectroscopy (GC/MS), or liquid chromatography/mass spectroscopy (LC/MS). In some embodiments, substantially free may be less than about 1.0%, less than about 0.5%, less than about 0.4%, less than about 0.3%, less than about 0.2%, less than about 0.1%, less than about 0.05%, or less than about 0.01%.

The term “interconversion” or “conformational interconversion” refers to any change between the atropisomers of this disclosure, including but not limited to equilibration.

The term “equilibration” refers to a chemical reaction in which the forward and reverse ratio rates cancel out. Equilibration can be dynamic or static. A reaction in equilibrium need not contain equal parts reactant and product. When referring to atropisomeric compounds, the term “equilibration” refers to when the rate of interconversion cancels out. Atropisomers in equilibrium need not contain equal parts of each single atropisomer and encompasses racemic mixtures of atropisomers, enriched mixtures of atropisomers, as well as single atropisomers.

Also provided are embodiments wherein any embodiment herein may be combined with any one or more of the other embodiments, unless otherwise stated and provided the combination is not mutually exclusive.

Atropisomers are stereoisomers resulting from hindered rotation about single bonds where the steric strain barrier to rotation is high enough to allow for the isolation of the conformers. Oki (Oki, M; Topics in Stereochemistry 1983, 1) defined atropisomers as conformers that interconvert with a half-life of more than 1000 seconds at a given temperature. The scope of embodiments herein as described and claimed encompasses the racemic forms of the compounds as well as the individual atropisomers (an atropisomer “substantially free” of its corresponding atropisomer) and stereoisomer-enriched mixtures, i.e. mixtures of atropisomers. Separation of atropisomers is possibly by chiral separation methods such as selective crystallization. In an atropo-enantioselective or atroposelective synthesis one atropisomer is formed at the expense of the other. Atroposelective synthesis may be carried out by use of chiral auxiliaries like a Corey-Bakshi-Shibata (CBS) catalyst (asymmetric catalyst derived from proline) in the total synthesis of knipholone or by approaches based on thermodynamic equilibration when an isomerization reaction favors one atropisomer over the other.

The term “atropisomerism” refers to a type of isomerism resulting from hindered rotation around a single bond due to steric strain of the substituents. This phenomenon creates stereoisomers which display axial chirality.

The following scheme illustrates “atropisomerism” with reference to specific pyridinone-pyridine compounds of the invention:

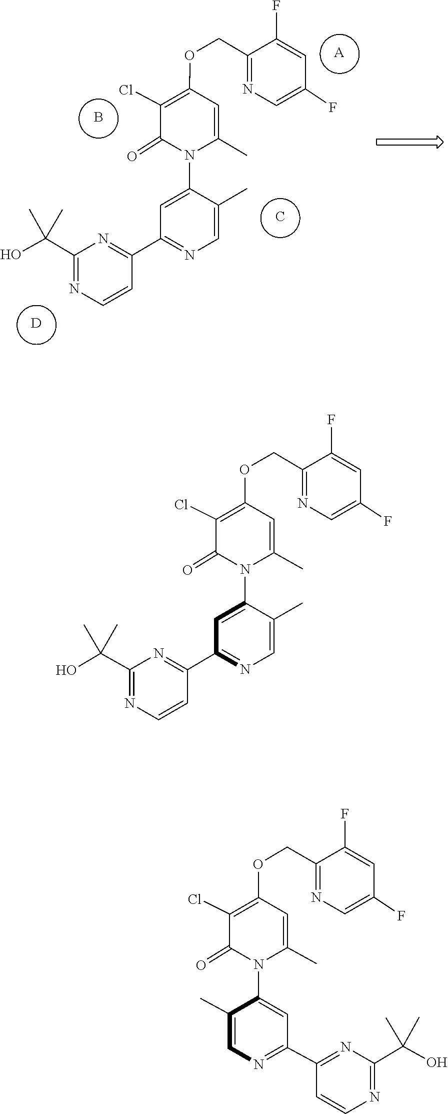

The bond between the B and C rings of the title compounds is hindered and does not allow for facile rotation. The steric strain barrier to rotation is sufficiently high such that individual conformers can be isolated. The compounds of the invention may also exist as atropisomers, i.e., chiral rotational isomers. The invention encompasses racemates, resolved atropisomers, and mixtures thereof. Atropisomers may be separated by a variety of chromatographic methods, including by not limited to supercritical fluid chromatography using a mobile phase of carbon dioxide and ethanol/methanol as well as simulated moving bed (SMB) chromatography with a chiral stationary phase and a mobile phase.

Atropisomers are generally stable but can often be equilibrated thermally. Atropisomers will have the same but opposite optical rotation. Each atropisomers may have different properties when bound to an enzyme or receptor with one isomer often being more potent than the other. Atropisomers are frequently used as pharmaceutical agents. Known examples include Vancomycin and derivatives.

The configuration of atropisomers can be described using the nomenclature (M)- and (P)- to describe the relative position of substituents as described in Bringmann, G. et. al., Angew. Chem. Int. Ed. 2005, 44, 5384 and references cited therein. Structures are designated as drawn but it is understood that either (P)- or (M)-isomers may be desirable and the methods described would be useful for the interconversion of either (P)- or (M)-stereoisomers.

### DETAILED DESCRIPTION

The present disclosure includes embodiments directed to methods of synthesizing a compound of Formula (P)-I, having the structure:

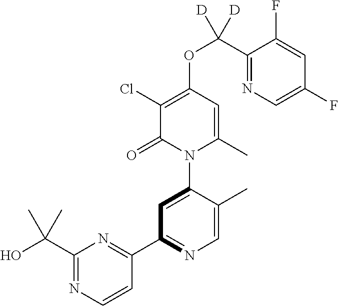

### (P)-3-chloro-4-((3,5-difluoropyridin-2-yl)methoxy-d₂)-2′-(2-(2-hydroxypropan-2-yl)pyrimidin-4-yl)-5′,6-dimethyl-2H-[1,4′-bipyridin]-2-one

Scheme 1 outlines a route for synthesizing a compound of Formula (P)-I. The route features a chiral separation as the last step of the synthesis of a compound of Formula (P)-I.

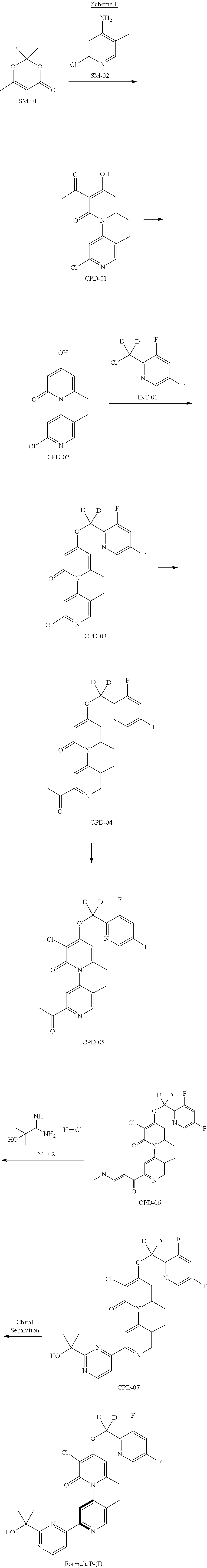

Scheme 2 outlines a route for synthesizing a compound of Formula (P)-I. The routes feature chiral separation of an intermediate and then carrying forward a single or enriched atropisomer through the remainder of the synthesis of a compound of Formula (P)-I.

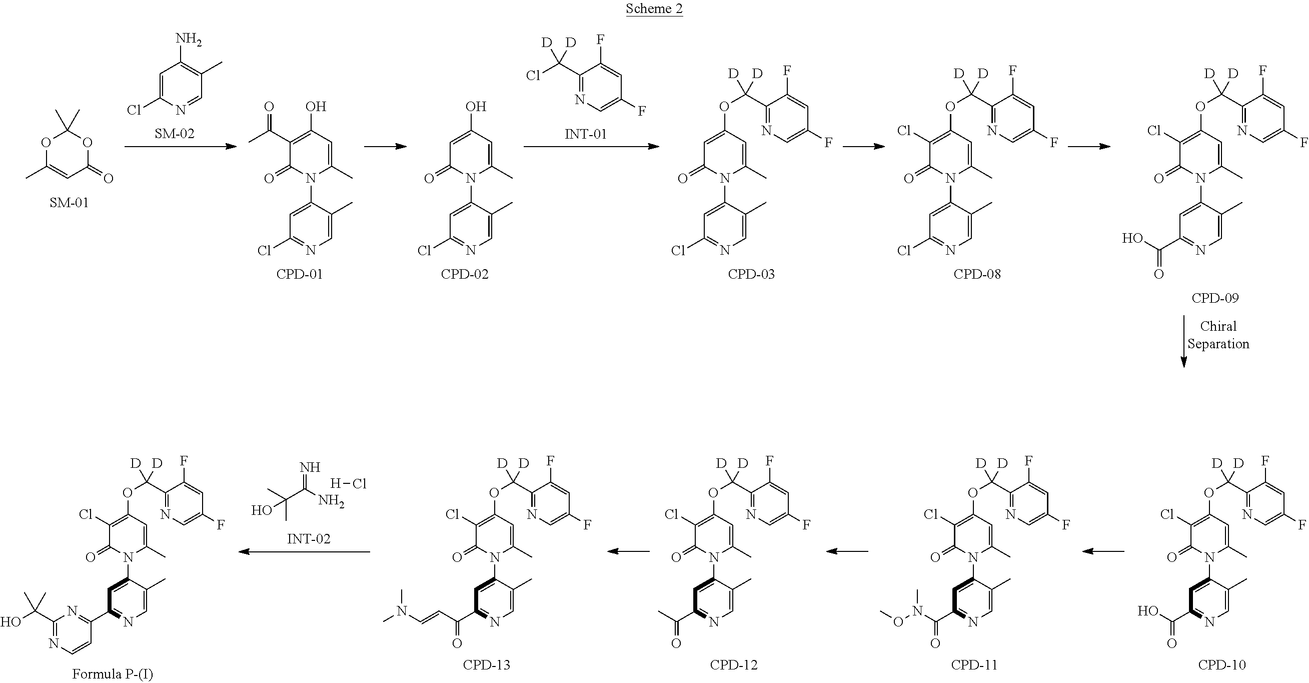

The present disclosure includes embodiments directed to methods of synthesizing a compound of Formula (P)-II, having the structure:

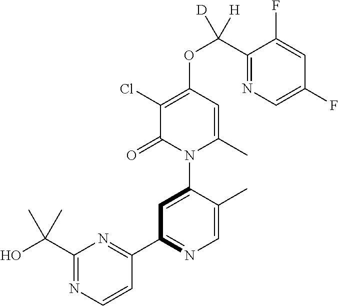

### (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one

Scheme 3 outlines a route for synthesizing a compound of Formula (P)-II. The route features a chiral separation as the last step of the synthesis of a compound of Formula (P)-II.

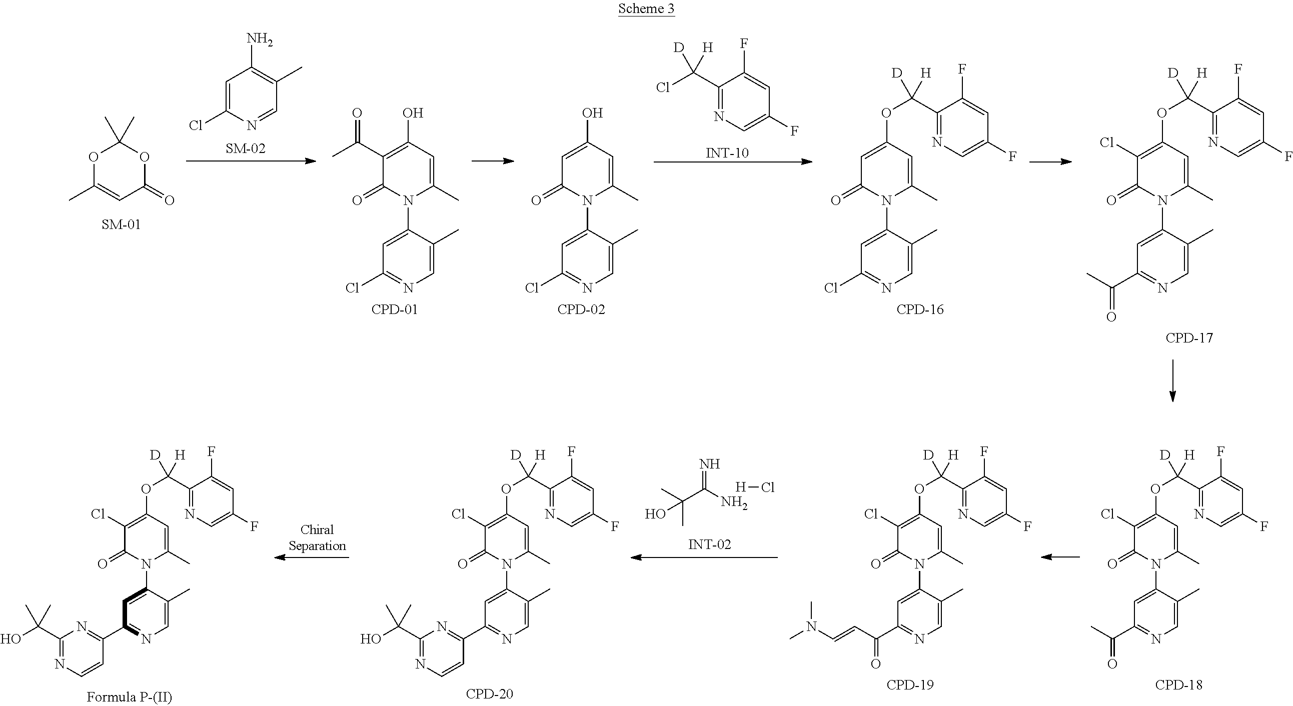

Scheme 4 outlines a route for synthesizing a compound of Formula (P)-II. The routes feature chiral separation of an intermediate and then carrying forward a single or enriched atropisomer through the remainder of the synthesis of a compound of Formula (P)-II.

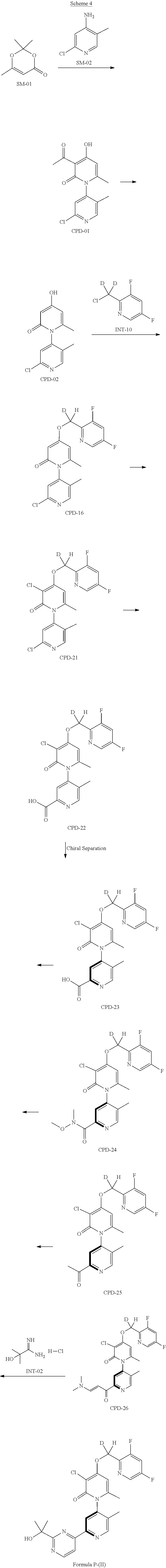

Some embodiments of the present application describe a process for the preparation of compound of Formula (P)-I having the structure:

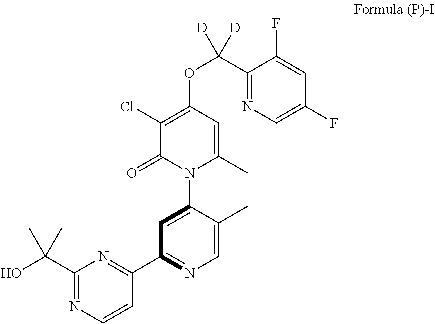

comprising the steps of:
(a) contacting the compound

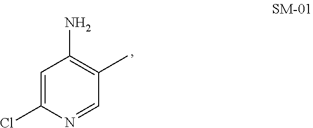

with the compound

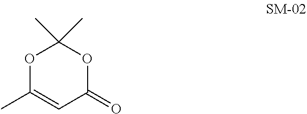

in the presence of dimethylacetemide (DMAc) to form a mixture; and
(b) contacting the mixture of (a) with an alcoholic HCl solution
to form the compound

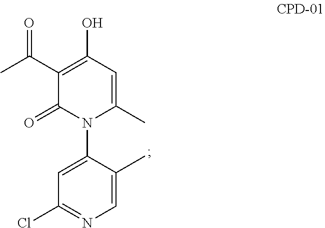 [SMILES: CC(=O)c1c(O)cc(C)n(-c2cc(Cl)ncc2C)c1=O]

and
(c) converting CPD-01 to Formula (P)-I.

In some embodiments of the forming of CPD-01, the alcoholic HCl solution is selected from the group consisting of an isopropyl alcohol HCl solution or p-toluenesulfonic acid in dimethylacetamide (DMAc).

In some embodiments of the forming of CPD-01, the alcoholic HCl solution is an isopropyl alcohol HCl solution.

In some embodiments of the process for the preparation of Formula (P)-I, the process further comprises contacting the compound CPD-01 with H₂SO₄ to form the compound

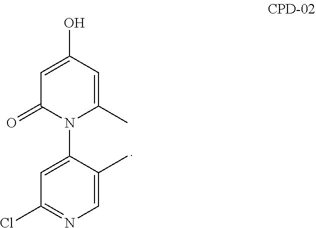 [SMILES: Cc1cnc(Cl)cc1-n1c(C)cc(O)cc1=O]

In some embodiments of the process for the preparation of Formula (P)-I, the process further comprises contacting the compound CPD-02 with the compound

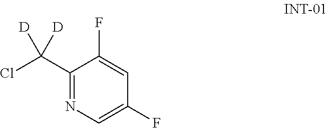 [SMILES: [2H]C([2H])(Cl)c1ncc(F)cc1F]

and a base to form the compound

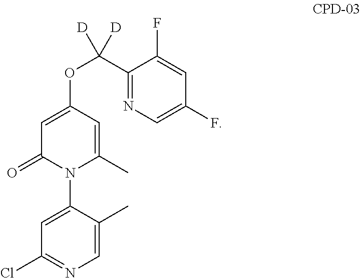 [SMILES: [2H]C([2H])(Oc1cc(C)n(-c2cc(Cl)ncc2C)c(=O)c1)c1ncc(F)cc1F]

In some embodiments of forming CPD-03, the base is selected from the group consisting of K₂CO₃, NaOH, Cs₂CO₃, and NaHCO₃.

In some embodiments, the base used to form CPD-03 is selected from the group consisting of K₂CO₃ and Cs₂CO₃.

In some embodiments, the base used to form CPD-03 is K₂CO₃.

In some embodiments, the base used to form CPD-03 is Cs₂CO₃.

In some embodiments of the process for the preparation of Formula (P)-I, the process further comprises the steps of:
(a) contacting the compound CPD-03 with a vinyl tin reagent in the presence of a copper catalyst and a palladium catalyst to form a mixture; and
(b) contacting the mixture of (a) with HCl
to form the compound

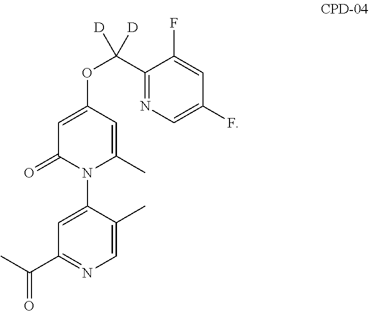 [SMILES: [2H]C([2H])(Oc1cc(C)n(-c2cc(C(C)=O)ncc2C)c(=O)c1)c1ncc(F)cc1F]

In some embodiments of forming CPD-04, the vinyl tin reagent is

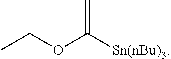

In some embodiments of forming CPD-04, the copper catalyst is CuI.

In some embodiments of forming CPD-04, the palladium catalyst is Pd(dppf)Cl₂.

In some embodiments of the process for the preparation of Formula (P)-I, the process further comprises contacting the compound CPD-02 with HBr to form the compound

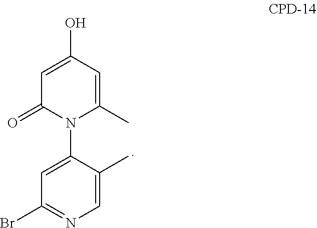 [SMILES: Cc1cnc(Br)cc1-n1c(C)cc(O)cc1=O]

In another embodiment of the process for the preparation of Formula (P)-I, the process further comprises contacting the compound CPD-14 with the compound

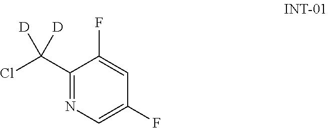 [SMILES: [2H]C([2H])(Cl)c1ncc(F)cc1F]

and a base to form the compound

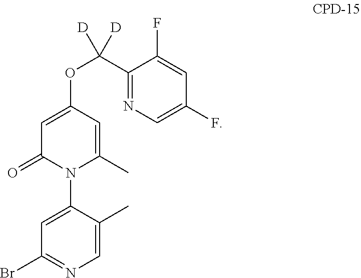 [SMILES: [2H]C([2H])(Oc1cc(C)n(-c2cc(Br)ncc2C)c(=O)c1)c1ncc(F)cc1F]

In some embodiments, the base used to form CPD-15 is selected from the group consisting of K₂CO₃ and Cs₂CO₃.

In some embodiments, the base used to form CPD-15 is K₂CO₃.

In some embodiments, the base used to form CPD-15 is Cs₂CO₃.

In some embodiments of the process for the preparation of Formula (P)-I, the process further comprises the steps of:
(a) contacting the compound CPD-15 with a vinyl tin reagent in the presence of a copper catalyst and a palladium catalyst to form a mixture; and
(b) contacting the mixture of (a) with an acid
to form the compound

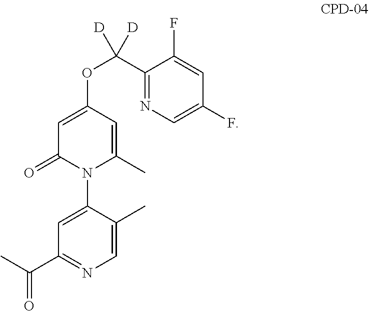 [SMILES: [2H]C([2H])(Oc1cc(C)n(-c2cc(C(C)=O)ncc2C)c(=O)c1)c1ncc(F)cc1F]

In some embodiments of forming CPD-04, the vinyl tin reagent is

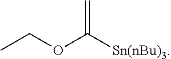

In some embodiments of forming CPD-04, the copper catalyst is CuI.

In some embodiments of forming CPD-04, the palladium catalyst is Pd(dppf)Cl₂.

In some embodiments of forming CPD-04, the acid is HCl.

In some embodiments of the process for the preparation of Formula (P)-I, the process further comprises contacting the compound CPD-04 prepared from any embodiment disclosed herein with a chlorination reagent to form the compound

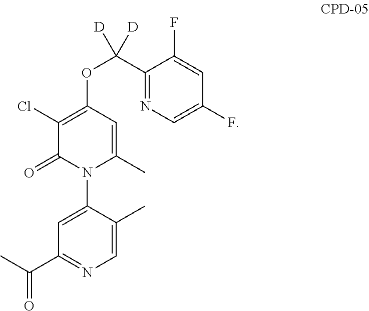 [SMILES: [2H]C([2H])(Oc1cc(C)n(-c2cc(C(C)=O)ncc2C)c(=O)c1Cl)c1ncc(F)cc1F]

In some embodiments of the forming of CPD-05, the chlorination reagent is N-chlorosuccinimide.

In some embodiments, the forming of CPD-05 further comprises contacting CPD-04 with dichloroacetic acid.

In some embodiments of the process for the preparation of Formula (P)-I, the process further comprises condensing the compound CPD-05 with N,N-dimethyl-formamide dimethyl acetal to obtain the compound

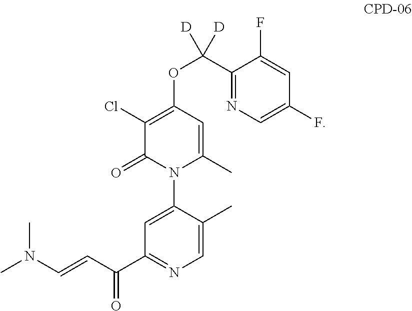 [SMILES: [2H]C([2H])(Oc1cc(C)n(-c2cc(C(=O)/C=C/N(C)C)ncc2C)c(=O)c1Cl)c1ncc(F)cc1F]

In some embodiments of the condensation of the compound CPD-05, the condensing further comprises L-proline.

In some embodiments of the process for the preparation of Formula (P)-I, the process further comprises contacting the compound CPD-06 with

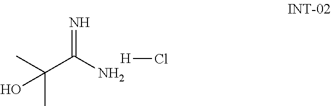

in the presence of a base, and
forming the compound

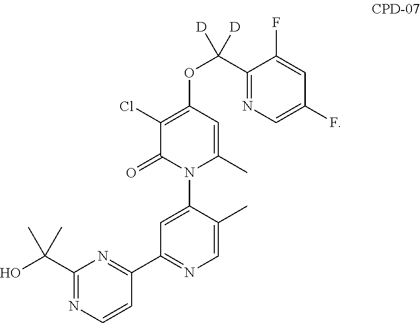 [SMILES: [2H]C([2H])(Oc1cc(C)n(-c2cc(-c3ccnc(C(C)(C)O)n3)ncc2C)c(=O)c1Cl)c1ncc(F)cc1F]

In some embodiments of forming the compound of CPD-07, the base is selected from the group consisting of K₂CO₃, N,N-diisopropylethylamine (DIPEA), triethylamine (TEA), tBuOK, tBuONa, and Cs₂CO₃.

In some embodiments of the forming of CPD-07, the base is K₂CO₃.

In some embodiments of the process for the preparation of Formula (P)-I, the process further comprises subjecting the compound CPD-07 to a chromatographic separation to obtain the compound of Formula (P)-I.

In some embodiments of the process for the preparation of Formula (P)-I, the chromatographic separation comprises supercritical fluid chromatography (SFC) using a SFC mobile phase.

In some embodiments of the process for the preparation of Formula (P)-I, the SFC mobile phase is carbon dioxide and isopropanol.

In some embodiments of the process for the preparation of Formula (P)-I, the chromatographic separation comprises simulated moving bed (SMB) chromatography with a SMB chiral stationary phase and a SMB mobile phase.

In some embodiments of the process for the preparation of Formula (P)-I, the SMB chiral stationary phase is selected from the group consisting of Chiralpak® AD, Chiralpak® AS, Chiralpak® AY, Chiralpak® AZ, Chiralpak® OD, Chiralpak® OZ, Chiralpak® IA, Chiralpak® IB-N, Chiralpak® IC, Chiralpak® ID, Chiralpak® IE, Chiralpak® IF, Chiralpak® IG, and Chiralpak® IH.

In some embodiments of the process for the preparation of Formula (P)-I, the SMB chiral stationary phase is Chiralpak® IB-N.

In some embodiments of the process for the preparation of Formula (P)-I, the SMB mobile phase is selected from the group consisting of acetonitrile, methanol, acetonitrile and methanol, n-heptane and ethanol, n-heptane and dichloromethane, n-heptane and ethylacetate, dichloromethane and methanol, and dichloromethane and acetonitrile.

In some embodiments of the process for the preparation of Formula (P)-I, the SMB mobile phase is dichloromethane and acetonitrile.

In some embodiments of the process for the preparation of Formula (P)-I, the SMB mobile phase is acetonitrile and methanol.

In some embodiments of the process for the preparation of Formula (P)-I, when the SMB mobile phase is in the form of a mixture the mixtures may be in a volumetric ratio of about 1:1, about 2:1, about 3:1, about 4:1, about 5:1, about 6:1, about 7:1, about 8:1, about 9:1, about 10:1, about 7:3, about 1:2, about 1:3, about 1:4, about 1:5, about 1:6, about 1:7, about 1:8, about 1:9, about 1:10, about 3:7, or any ratio in between any two ratios.

In another embodiment of the process for the preparation of Formula (P)-I, the process further comprises contacting the compound CPD-03 with a chlorination reagent to form the compound

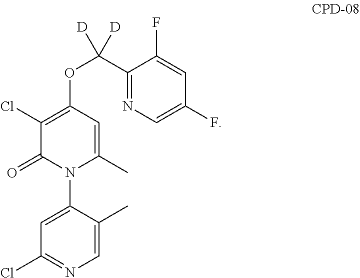 [SMILES: [2H]C([2H])(Oc1cc(C)n(-c2cc(Cl)ncc2C)c(=O)c1Cl)c1ncc(F)cc1F]

In some embodiments of forming CPD-08, the chlorination reagent is N-chlorosuccinimide.

In some embodiments, the forming of CPD-08 further comprises contacting CPD-03 with dichloroacetic acid.

In some embodiments of the process for the preparation of Formula (P)-I, the process further comprises contacting the compound CPD-08 with CO in the presence of a palladium catalyst, a base, and a solvent mixture to form the compound

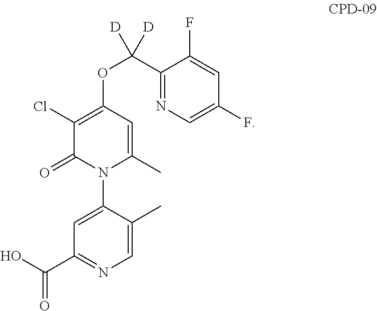 [SMILES: [2H]C([2H])(Oc1cc(C)n(-c2cc(C(=O)O)ncc2C)c(=O)c1Cl)c1ncc(F)cc1F]

In some embodiments of forming CPD-09, the palladium catalyst is Pd(dppf)Cl₂.

In some embodiments of forming CPD-09, the base is Na₂CO₃.

In some embodiments of forming CPD-09, the base is K₂CO₃.

In some embodiments of forming CPD-09, the base is Li₂CO₃.

In some embodiments of forming CPD-09, the forming of CPD-09 further comprising contacting CPD-08 with triethylamine.

In some embodiments of forming CPD-09, the solvent mixture is MeOH/H₂O.

In some embodiments of forming CPD-09, the solvent mixture is acetonitrile/H₂O.

In some embodiments of the process for the preparation of Formula (P)-I, the process further comprises subjecting the compound CPD-09 to chiral separation with a chiral amine and a solvent to obtain the compound

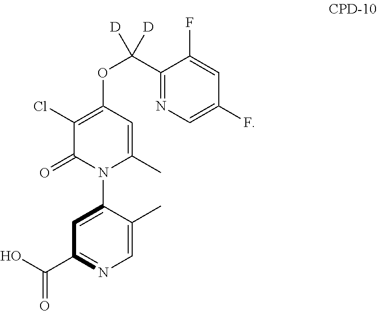

In some embodiments of the chiral separation of the compound CPD-10, the chiral amine is selected from the group consisting of (5)-1-(naphthalen-2-yl)ethan-1-amine and (1S, 2R)-2-amino-1,2-diphenylethan-1-ol.

In some embodiments of the chiral separation of the compound CPD-10, the chiral amine is (5)-1-(naphthalen-2-yl)ethan-1-amine.

In some embodiments of the chiral separation of the compound CPD-10, the chiral amine is (1S, 2R)-2-amino-1,2-diphenylethan-1-ol.

In some embodiments of the chiral separation of the compound CPD-10, the solvent is selected from the group consisting of toluene, ethylbenzene, n-butanol, anisole, DMSO, or a combination thereof.

In some embodiments of the chiral separation of the compound CPD-10, the solvent is toluene.

In some embodiments of the chiral separation of the compound CPD-10, the solvent is ethylbenzene.

In some embodiments of the chiral separation of the compound CPD-10, the solvent is n-butanol.

In some embodiments of the chiral separation of the compound CPD-10, the solvent is anisole.

In some embodiments of the chiral separation of the compound CPD-10, the solvent is anisole and DMSO.

In some embodiments of the process for the preparation of Formula (P)-I, the process further comprises contacting the compound CPD-10 with a solvent, MeNHOMe, an amine base, and a coupling reagent to obtain the compound

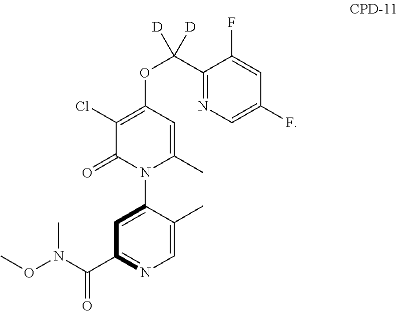

In some embodiments of contacting the compound CPD-10, the solvent is selected from DMF, dichloromethane, or a combination thereof.

In some embodiments of contacting the compound CPD-10, the solvent is DMF.

In some embodiments of contacting the compound CPD-10, the solvent is dichloromethane.

In some embodiments of the contacting the compound CPD-10, the amine base is triethylamine.

In some embodiments of the contacting the compound CPD-10, the coupling reagent is N-(3-Dimethylaminopropyl)-N′-ethylcarbodiimide hydrochloride.

In some embodiments of the process for the preparation of Formula (P)-I, the process further comprises contacting the compound CPD-11 with MeMgX to obtain the compound

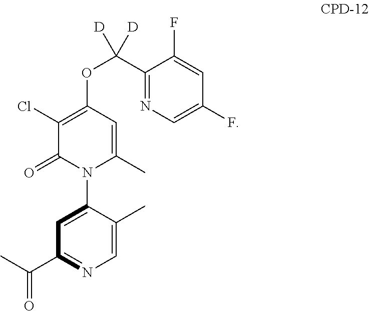

In some embodiments of the contacting the compound CPD-11, the MeMgX is selected from the group consisting of MeMgBr and MeMgCl.

In some embodiments of the contacting the compound CPD-11, the MeMgX is MeMgBr.

In some embodiments of the contacting the compound CPD-11, the MeMgX is MeMgCl.

In some embodiments of the process for the preparation of Formula (P)-I, the process further comprises condensing compound CPD-12 with N,N-dimethyl-formamide dimethyl acetal to obtain the compound

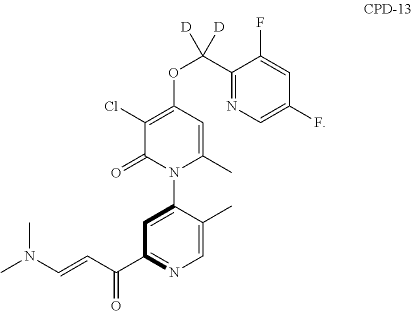

In some embodiments of the process for the preparation of Formula (P)-I, the process further comprises contacting the compound CPD-13 with

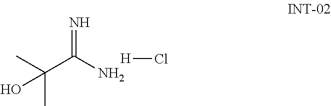

in the presence of a base, and forming the compound of Formula (P)-I.

In some embodiments of the process for the preparation of Formula (P)-I, the process

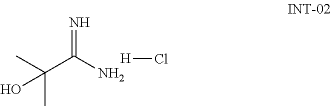

further comprises contacting the compound CPD-13 with in the presence of a base, forming the crude compound of Formula (P)-I, and crystallizing the crude compound of Formula (P)-I with a crystallization solvent mixture to yield a crystallized compound of Formula (P)-I.

In some embodiments of the contacting the compound CPD-13, the base is selected from the group consisting of K₂CO₃, N,N-diisopropylethylamine (DIPEA), triethylamine (TEA), tBuOK, tBuONa, and Cs₂CO₃.

In some embodiments of the contacting the compound CPD-13, the base is K₂CO₃.

In some embodiments of the contacting the compound CPD-13, the crystallization solvent mixture is water and 1-propanol.

In some embodiments of the contacting the compound CPD-26, the crystallization solvent mixture is water and methanol.

In some embodiments of the contacting the compound CPD-13, the base is selected from the group consisting of K₂CO₃, N,N-diisopropylethylamine (DIPEA), triethylamine (TEA), tBuOK, tBuONa, and Cs₂CO₃.

In some embodiments of the contacting the compound CPD-13, the base is K₂CO₃.

Scheme 5 depicts a method of synthesizing INT-01 starting from INT-A. INT-01 produced in this manner may be used in any of the embodiments disclosed herein that utilizes INT-01.

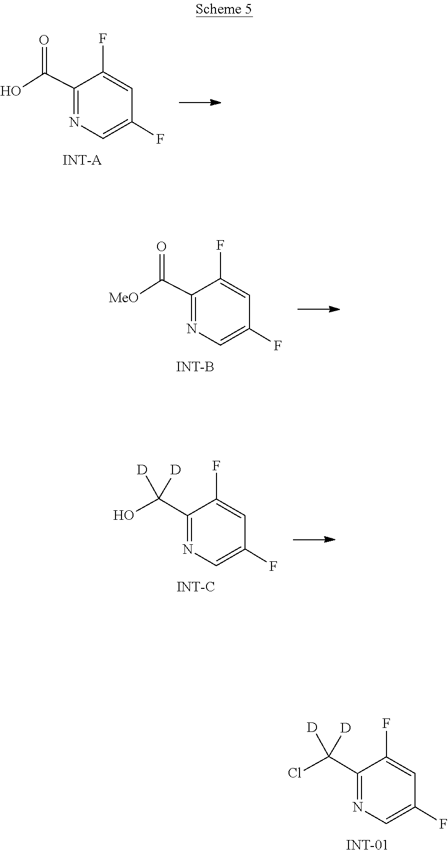

In accordance with Scheme 5, another embodiment of the present application involve a process for the preparation of compound INT-01 comprising contacting the compound INT-A with SOCl₂ and methanol to form the compound INT-B and converting INT-B to INT-01.

In some embodiments of the process for the preparation of compound INT-01, the process further comprises contacting the compound INT-B with NaBD₄, ZnCl₂, and a solvent to form the compound INT-C.

In some embodiments of the contacting the compound INT-B, the solvent is selected from THF and THF-dg.

In some embodiments of the process for the preparation of compound INT-01, the process further comprises contacting the compound INT-C with SOCl₂ and a solvent to form the compound INT-01.

In some embodiments of the contacting the compound INT-C, the solvent is selected from DCM and DCM-d₂.

Some embodiments of the present application describe a process for the preparation of compound of Formula (P)-II having the structure:

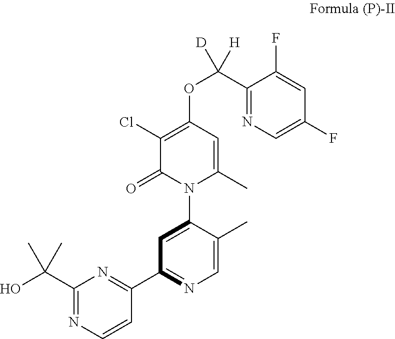

comprising the steps of:
(a) contacting the compound

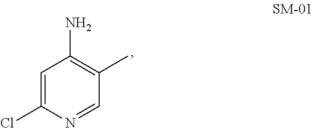

with the compound

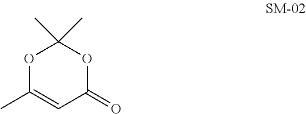

in the presence of dimethylacetemide (DMAc) to form a mixture; and
(b) contacting the mixture of (a) with an alcoholic HCl solution
to form the compound

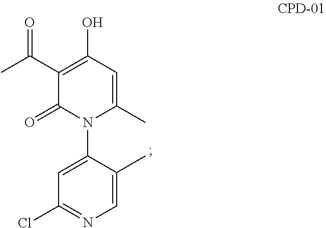 [SMILES: CC(=O)c1c(O)cc(C)n(-c2cc(Cl)ncc2C)c1=O]

and
(c) converting CPD-01 to Formula (P)-II.

In some embodiments of the forming of CPD-01, the alcoholic HCl solution is selected from the group consisting of an isopropyl alcohol HCl solution or p-toluenesulfonic acid in dimethylacetamide (DMAc).

In some embodiments of the forming of CPD-01, the alcoholic HCl solution is an isopropyl alcohol HCl solution.

In some embodiments of the process for the preparation of Formula (P)-II, the process further comprises contacting the compound CPD-01 with H₂SO₄ to form the compound

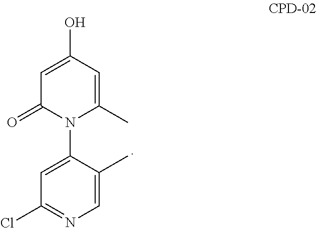 [SMILES: Cc1cnc(Cl)cc1-n1c(C)cc(O)cc1=O]

In some embodiments of the process for the preparation of Formula (P)-II, the process further comprises contacting the compound CPD-02 with the compound

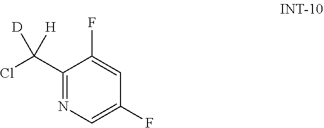 [SMILES: [2H]C(Cl)c1ncc(F)cc1F]

and a base to form the compound

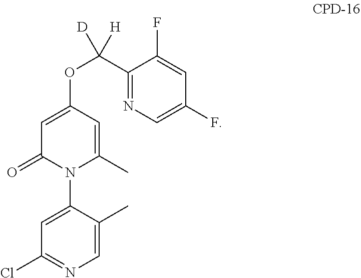 [SMILES: [2H]C(Oc1cc(C)n(-c2cc(Cl)ncc2C)c(=O)c1)c1ncc(F)cc1F]

In some embodiments of forming CPD-16, the base is selected from the group consisting of K₂CO₃, NaOH, Cs₂CO₃, and NaHCO₃.

In some embodiments, the base used to form CPD-16 is selected from the group consisting of K₂CO₃ and Cs₂CO₃.

In some embodiments, the base used to form CPD-16 is K₂CO₃.

In some embodiments, the base used to form CPD-16 is Cs₂CO₃.

In some embodiments of the process for the preparation of Formula (P)-II, the process further comprises the steps of:
(a) contacting the compound CPD-16 with a vinyl tin reagent in the presence of a copper catalyst and a palladium catalyst to form a mixture; and
(b) contacting the mixture of (a) with HCl
to form the compound

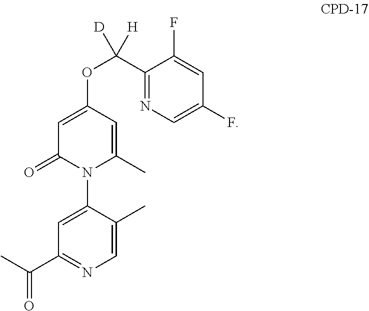 [SMILES: [2H]C(Oc1cc(C)n(-c2cc(C(C)=O)ncc2C)c(=O)c1)c1ncc(F)cc1F]

In some embodiments of forming CPD-17, the vinyl tin reagent is

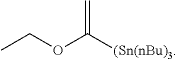

In some embodiments of forming CPD-17, the copper catalyst is CuI.

In some embodiments of forming CPD-17, the palladium catalyst is Pd(dppf)Cl₂.

In some embodiments of the process for the preparation of Formula (P)-II, the process further comprises contacting the compound CPD-02 with HBr to form the compound

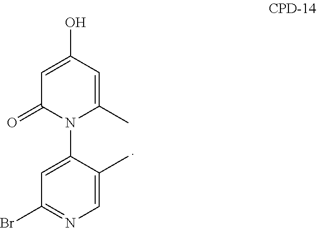 [SMILES: Cc1cnc(Br)cc1-n1c(C)cc(O)cc1=O]

In another embodiment of the process for the preparation of Formula (P)-II, the process further comprises contacting the compound CPD-14 with the compound

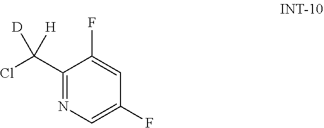 [SMILES: [2H]C(Cl)c1ncc(F)cc1F]

and a base to form the compound

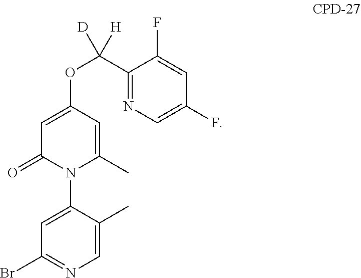 [SMILES: [2H]C(Oc1cc(C)n(-c2cc(Br)ncc2C)c(=O)c1)c1ncc(F)cc1F]

In some embodiments, the base used to form CPD-27 is selected from the group consisting of K₂CO₃ and Cs₂CO₃.

In some embodiments, the base used to form CPD-27 is K₂CO₃.

In some embodiments, the base used to form CPD-27 is Cs₂CO₃.

In some embodiments of the process for the preparation of Formula (P)-II, the process further comprises the steps of:
(a) contacting the compound CPD-27 with a vinyl tin reagent in the presence of a copper catalyst and a palladium catalyst to form a mixture; and
(b) contacting the mixture of (a) with an acid
to form the compound

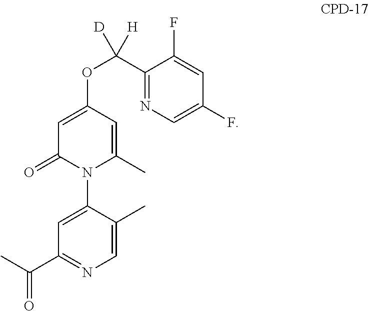 [SMILES: [2H]C(Oc1cc(C)n(-c2cc(C(C)=O)ncc2C)c(=O)c1)c1ncc(F)cc1F]

In some embodiments of forming CPD-17, the vinyl tin reagent is

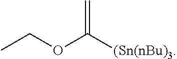

In some embodiments of forming CPD-17, the copper catalyst is CuI.

In some embodiments of forming CPD-17, the palladium catalyst is Pd(dppf)Cl₂.

In some embodiments of forming CPD-17, the acid is HCl.

In some embodiments of the process for the preparation of Formula (P)-II, the process further comprises contacting the compound CPD-17 prepared from any embodiment disclosed herein with a chlorination reagent to form the compound

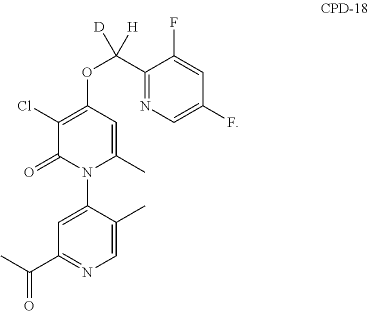 [SMILES: [2H]C(Oc1cc(C)n(-c2cc(C(C)=O)ncc2C)c(=O)c1Cl)c1ncc(F)cc1F]

In some embodiments of the forming of CPD-18, the chlorination reagent is N-chlorosuccinimide.

In some embodiments, the forming of CPD-18 further comprises contacting CPD-17 with dichloroacetic acid.

In some embodiments of the process for the preparation of Formula (P)-II, the process further comprises condensing the compound CPD-18 with N,N-dimethyl-formamide dimethyl acetal to obtain the compound

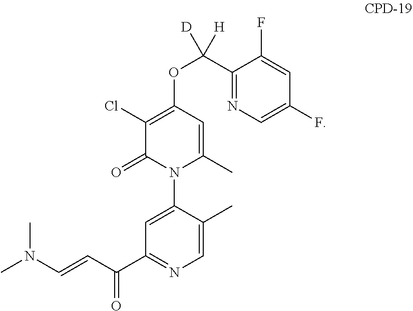 [SMILES: [2H]C(Oc1cc(C)n(-c2cc(C(=O)/C=C/N(C)C)ncc2C)c(=O)c1Cl)c1ncc(F)cc1F]

In some embodiments of the condensation of the compound CPD-18, the condensing further comprises L-proline.

In some embodiments of the process for the preparation of Formula (P)-II, the process further comprises contacting the compound CPD-19 with

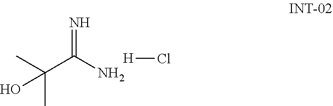

in the presence of a base, and
forming the compound

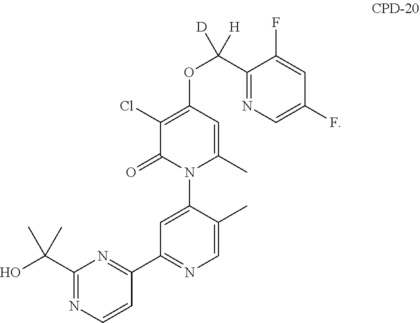 [SMILES: [2H]C(Oc1cc(C)n(-c2cc(-c3ccnc(C(C)(C)O)n3)ncc2C)c(=O)c1Cl)c1ncc(F)cc1F]

In some embodiments of forming the compound of CPD-20, the base is selected from the group consisting of K₂CO₃, N,N-diisopropylethylamine (DIPEA), triethylamine (TEA), tBuOK, tBuONa, and Cs₂CO₃.

In some embodiments of the forming of CPD-20, the base is K₂CO₃.

In some embodiments of the process for the preparation of Formula (P)-II, the process further comprises subjecting the compound CPD-20 to a chromatographic separation to obtain the compound of Formula (P)-II.

In some embodiments of the process for the preparation of Formula (P)-II, the chromatographic separation comprises supercritical fluid chromatography (SFC) using a SFC mobile phase.

In some embodiments of the process for the preparation of Formula (P)-II, the SFC mobile phase is carbon dioxide and isopropanol.

In some embodiments of the process for the preparation of Formula (P)-II, the chromatographic separation comprises simulated moving bed (SMB) chromatography with a SMB chiral stationary phase and a SMB mobile phase.

In some embodiments of the process for the preparation of Formula (P)-II, the SMB chiral stationary phase is selected from the group consisting of Chiralpak® AD, Chiralpak® AS, Chiralpak® AY, Chiralpak® AZ, Chiralpak® OD, Chiralpak® OZ, Chiralpak® IA, Chiralpak® IB-N, Chiralpak® IC, Chiralpak® ID, Chiralpak® IE, Chiralpak® IF, Chiralpak® IG, and Chiralpak® IH.

In some embodiments of the process for the preparation of Formula (P)-II, the SMB chiral stationary phase is Chiralpak® IB-N.

In some embodiments of the process for the preparation of Formula (P)-II, the SMB mobile phase is selected from the group consisting of acetonitrile, methanol, acetonitrile and methanol, n-heptane and ethanol, n-heptane and dichloromethane, n-heptane and ethylacetate, dichloromethane and methanol, and dichloromethane and acetonitrile.

In some embodiments of the process for the preparation of Formula (P)-II, the SMB mobile phase is dichloromethane and acetonitrile.

In some embodiments of the process for the preparation of Formula (P)-II, the SMB mobile phase is acetonitrile and methanol.

In some embodiments of the process for the preparation of Formula (P)-II, when the SMB mobile phase is in the form of a mixture the mixtures may be in a volumetric ratio of about 1:1, about 2:1, about 3:1, about 4:1, about 5:1, about 6:1, about 7:1, about 8:1, about 9:1, about 10:1, about 7:3, about 1:2, about 1:3, about 1:4, about 1:5, about 1:6, about 1:7, about 1:8, about 1:9, about 1:10, about 3:7, or any ratio in between any two ratios.

In another embodiment of the process for the preparation of Formula (P)-II, the process further comprises contacting the compound CPD-16 with a chlorination reagent to form the compound

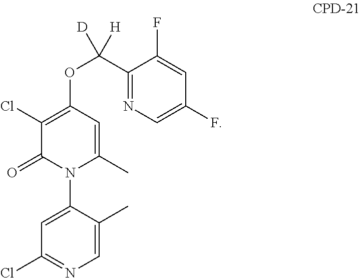 [SMILES: [2H]C(Oc1cc(C)n(-c2cc(Cl)ncc2C)c(=O)c1Cl)c1ncc(F)cc1F]

In some embodiments of forming CPD-21, the chlorination reagent is N-chlorosuccinimide.

In some embodiments, the forming of CPD-21 further comprises contacting CPD-16 with dichloroacetic acid.

In some embodiments of the process for the preparation of Formula (P)-II, the process further comprises contacting the compound CPD-21 with CO in the presence of a palladium catalyst, a base, and a solvent mixture to form the compound

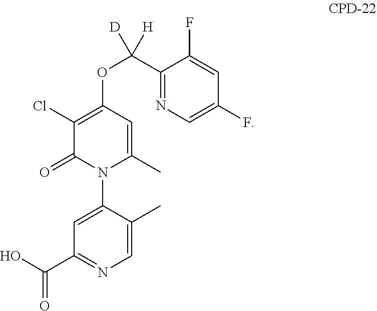 [SMILES: [2H]C(Oc1cc(C)n(-c2cc(C(=O)O)ncc2C)c(=O)c1Cl)c1ncc(F)cc1F]

In some embodiments of forming CPD-22, the palladium catalyst is Pd(dppf)Cl₂.

In some embodiments of forming CPD-22, the base is Na₂CO₃.

In some embodiments of forming CPD-22, the base is K₂CO₃.

In some embodiments of forming CPD-22, the base is Li₂CO₃.

In some embodiments of forming CPD-22, the forming of CPD-22 further comprising contacting CPD-21 with triethylamine.

In some embodiments of forming CPD-22, the solvent mixture is MeOH/H₂O.

In some embodiments of forming CPD-22, the solvent mixture is acetonitrile/H₂O.

In some embodiments of the process for the preparation of Formula (P)-II, the process further comprises subjecting the compound CPD-22 to chiral separation with a chiral amine and a solvent to obtain the compound

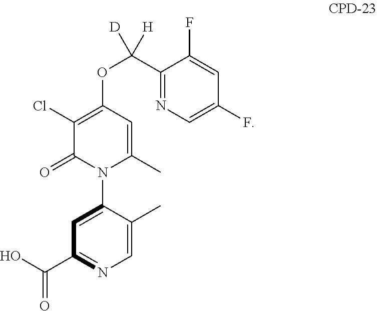

In some embodiments of the chiral separation of the compound CPD-23, the chiral amine is selected from the group consisting of (5)-1-(naphthalen-2-yl)ethan-1-amine and (1S, 2R)-2-amino-1,2-diphenylethan-1-ol.

In some embodiments of the chiral separation of the compound CPD-23, the chiral amine is (5)-1-(naphthalen-2-yl)ethan-1-amine.

In some embodiments of the chiral separation of the compound CPD-23, the chiral amine is (1S, 2R)-2-amino-1,2-diphenylethan-1-ol.

In some embodiments of the chiral separation of the compound CPD-23, the solvent is selected from the group consisting of toluene, ethylbenzene, n-butanol, anisole, DMSO, or a combination thereof.

In some embodiments of the chiral separation of the compound CPD-23, the solvent is toluene.

In some embodiments of the chiral separation of the compound CPD-23, the solvent is ethylbenzene.

In some embodiments of the chiral separation of the compound CPD-23, the solvent is n-butanol.

In some embodiments of the chiral separation of the compound CPD-23, the solvent is anisole.

In some embodiments of the chiral separation of the compound CPD-23, the solvent is anisole and DMSO.

In some embodiments of the process for the preparation of Formula (P)-II, the process further comprises contacting the compound CPD-23 with a solvent, MeNHOMe, an amine base, and a coupling reagent to obtain the compound

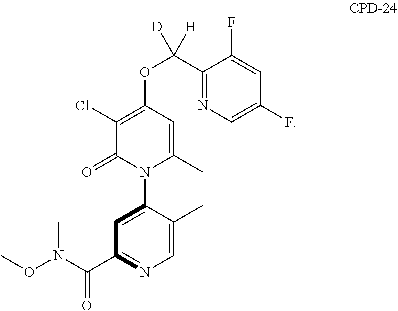

In some embodiments of contacting the compound CPD-23, the solvent is selected from DMF, dichloromethane, or a combination thereof.

In some embodiments of contacting the compound CPD-23, the solvent is DMF.

In some embodiments of contacting the compound CPD-23, the solvent is dichloromethane.

In some embodiments of the contacting the compound CPD-23, the amine base is triethylamine.

In some embodiments of the contacting the compound CPD-23, the coupling reagent is N-(3-Dimethylaminopropyl)-N′-ethylcarbodiimide hydrochloride.

In some embodiments of the process for the preparation of Formula (P)-II, the process further comprises contacting the compound CPD-24 with MeMgX to obtain the compound

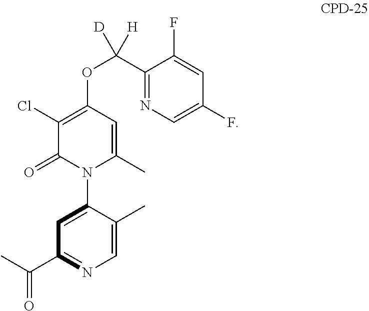

In some embodiments of the contacting the compound CPD-24, the MeMgX is selected from the group consisting of MeMgBr and MeMgCl.

In some embodiments of the contacting the compound CPD-24, the MeMgX is MeMgBr.

In some embodiments of the contacting the compound CPD-24, the MeMgX is MeMgCl.

In some embodiments of the process for the preparation of Formula (P)-II, the process further comprises condensing compound CPD-25 with N,N-dimethyl-formamide dimethyl acetal to obtain the compound

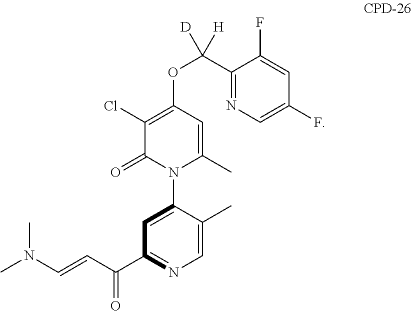

In some embodiments of the process for the preparation of Formula (P)-II, the process further comprises contacting the compound CPD-26 with

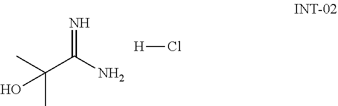

in the presence of a base, and forming the compound of Formula (P)-II.

In some embodiments of the process for the preparation of Formula (P)-II, the process further comprises contacting the compound CPD-26 with

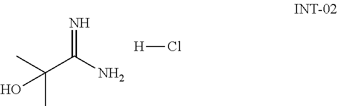

in the presence of a base, forming the crude compound of Formula (P)-II, and crystallizing the crude compound of Formula (P)-II with a crystallization solvent mixture to yield a crystallized compound of Formula (P)-II.

In some embodiments of the contacting the compound CPD-26, the base is selected from the group consisting of K₂CO₃, N,N-diisopropylethylamine (DIPEA), triethylamine (TEA), tBuOK, tBuONa, and Cs₂CO₃.

In some embodiments of the contacting the compound CPD-26, the base is K₂CO₃.

In some embodiments of the contacting the compound CPD-26, the crystallization solvent mixture is water and 1-propanol.

In some embodiments of the contacting the compound CPD-26, the crystallization solvent mixture is water and methanol.

Scheme 6 depicts a method of synthesizing INT-10 starting from INT-A. INT-10 produced in this manner may be used in any of the embodiments disclosed herein that utilizes INT-10.

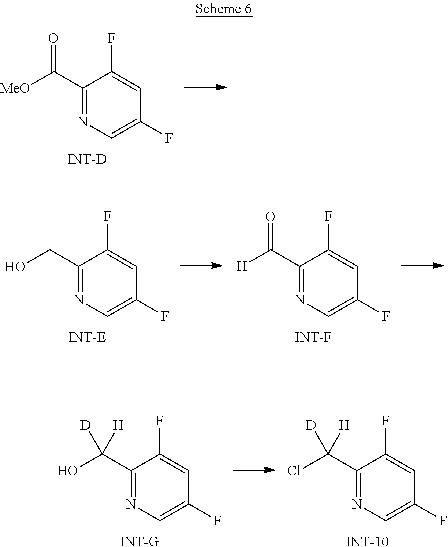

In accordance with Scheme 6, another embodiment of the present application involve a process for the preparation of compound INT-10 comprising contacting the compound INT-D with NaBH₄ and methanol to form the compound INT-E and converting INT-E to INT-10.

In some embodiments of the process for the preparation of compound INT-10, the process further comprises contacting the compound INT-E with Dess-Martin periodinane and DCM to form the compound INT-F.

In some embodiments of the process for the preparation of compound INT-10, the process further comprises contacting the compound INT-F with NaBD₄ and a solvent mixture to form the compound INT-G.

In some embodiments of the contacting the compound INT-F, the solvent mixture is selected from methanol/THF, methanol-d₄/THF, methanol-d₁/THF, methanol/THF-d₈, and methanol-d₁/THF-d₈, and methanol-d₄/THF-d₈.

In some embodiments of the process for the preparation of compound INT-10, the process further comprises contacting the compound INT-G with SOCl₂ and DCM to form the compound INT-10.

Some embodiments are of the present application describe a process for the preparation of compound of Formula (P)-A having the structure:

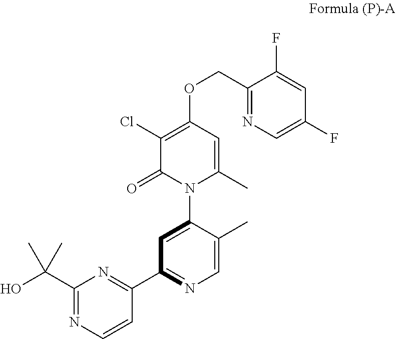

comprising:

contacting the compound

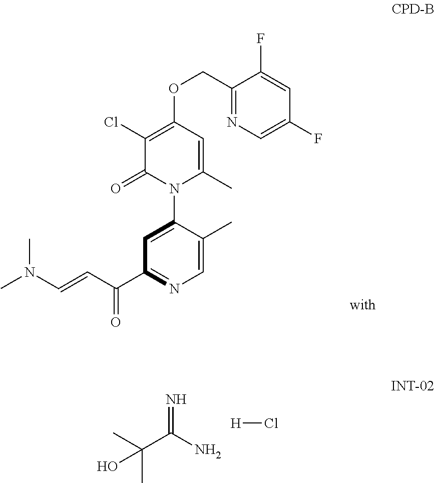

in the presence of a base, forming the crude compound of Formula (P)-A, and crystallizing the crude compound of Formula (P)-A with a crystallization solvent mixture to yield a crystallized compound of Formula (P)-A.

In some embodiments of the process for the preparation of compound of Formula (P)-A, the base is selected from the group consisting of K₂CO₃, N,N-diisopropylethylamine (DIPEA), triethylamine (TEA), tBuOK, tBuONa, and Cs₂CO₃.

In some embodiments of the process for the preparation of compound of Formula (P)-A, the base is K₂CO₃.

In some embodiments process for the preparation of compound of Formula (P)-A, the crystallization solvent mixture is water and 1-propanol.

In some embodiments process for the preparation of compound of Formula (P)-A, the crystallization solvent mixture is water and methanol.

CPD-B may be prepared by the methods disclosed in Example 10 Step 3 or Example 12 Step 3 of US2022/0235025A1, which is hereby incorporated by reference herein.

Some embodiments are directed towards a compound, or a salt thereof, or a co-crystal thereof, of the structure:

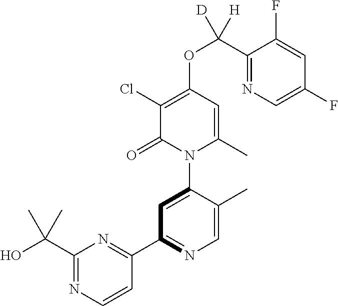

### (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one

Some embodiments are directed towards a compound, or a salt thereof, or a co-crystal thereof, of the structure:

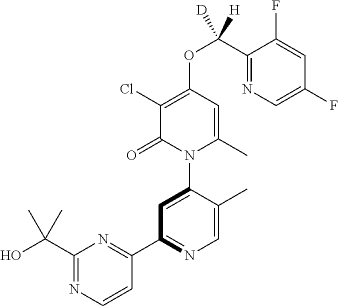

### (P)-(R)-3-chloro-4-((3,5-difluoropyridin-211)methoxy-d)-2′-(2-(2-hydroxypropan-2-yl)pyrimidin-4-yl)-5′,6-dimethyl-2H-[1,4′-bipyridin]-2-one

Some embodiments are directed towards a compound, or a salt thereof, or a co-crystal thereof, of the structure:

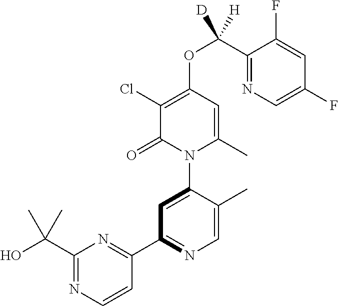

### (P)-(S)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one

Some embodiments are directed towards a compound, or a salt thereof, or a co-crystal thereof, of the structure:

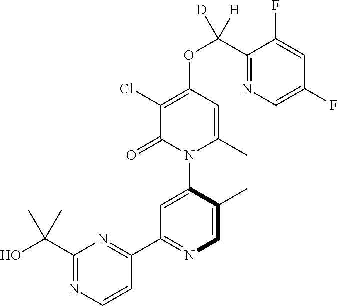

### (M)-3-chloro-4-((3,5-difluoropyridin-2-yl)methoxy-d)-2′-(2-(2-hydroxypropan-211)pyrimidin-411)-5′,6-dimethyl-2H-[1,4′-bipyridin]-2-one

Some embodiments are directed towards a compound, or a salt thereof, or a co-crystal thereof, of the structure:

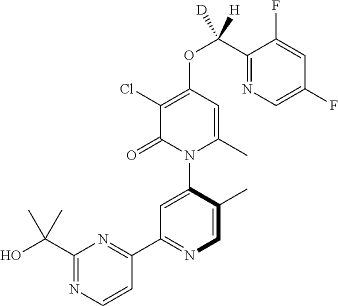

### (M)-(R)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one

Some embodiments are directed towards a compound, or a salt thereof, or a co-crystal thereof, of the structure:

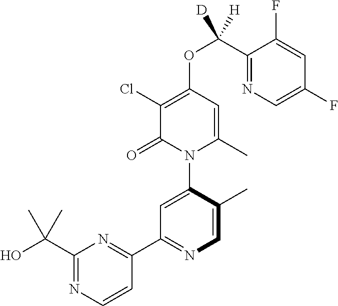

### (M)-(S)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one

Some embodiments of the present application relate to a compound, or a salt thereof, or a co-crystal thereof, selected from the group consisting of:

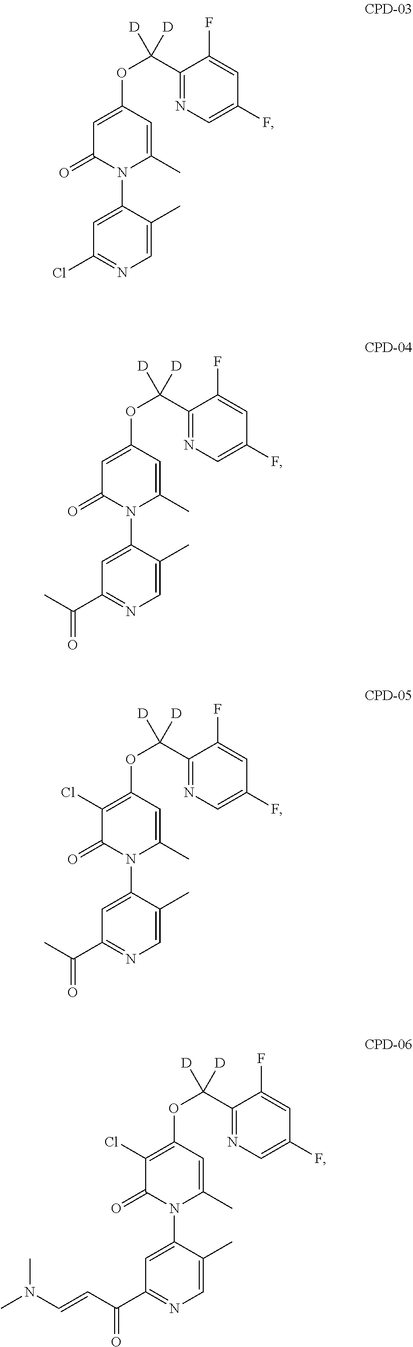 [SMILES: [2H]C([2H])(Oc1cc(C)n(-c2cc(Cl)ncc2C)c(=O)c1)c1ncc(F)cc1F ; [2H]C([2H])(Oc1cc(C)n(-c2cc(C(C)=O)ncc2C)c(=O)c1)c1ncc(F)cc1F ; [2H]C([2H])(Oc1cc(C)n(-c2cc(C(C)=O)ncc2C)c(=O)c1Cl)c1ncc(F)cc1F ; [2H]C([2H])(Oc1cc(C)n(-c2cc(C(=O)/C=C/N(C)C)ncc2C)c(=O)c1Cl)c1ncc(F)cc1F]

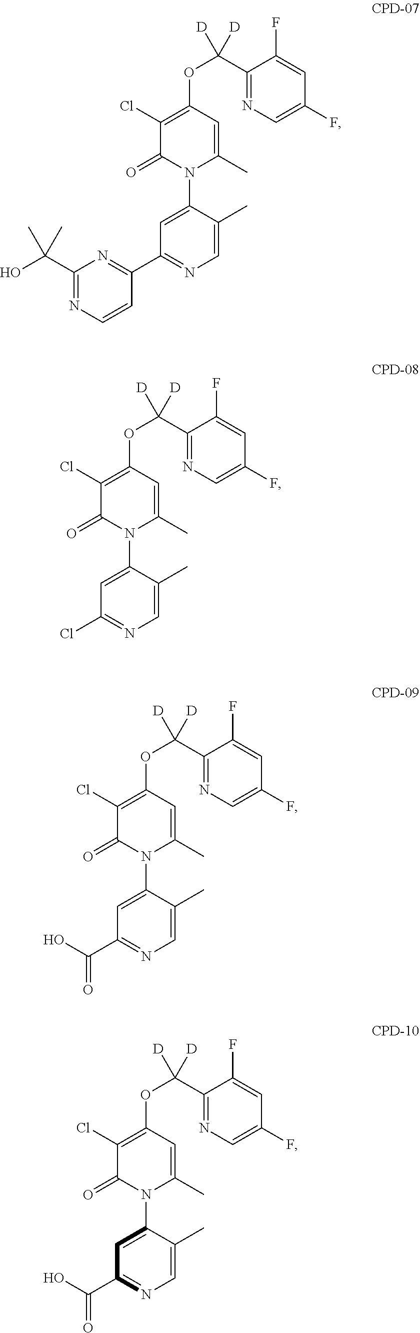

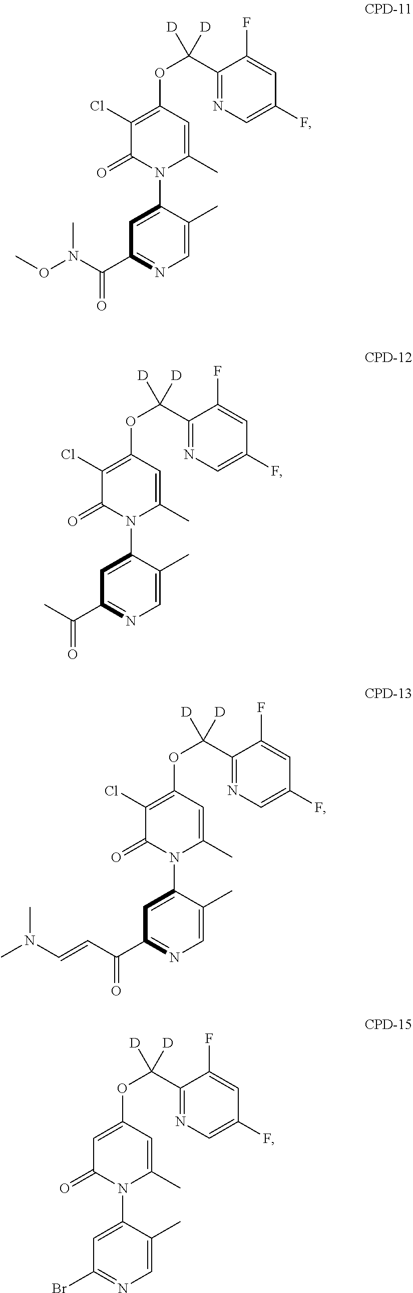

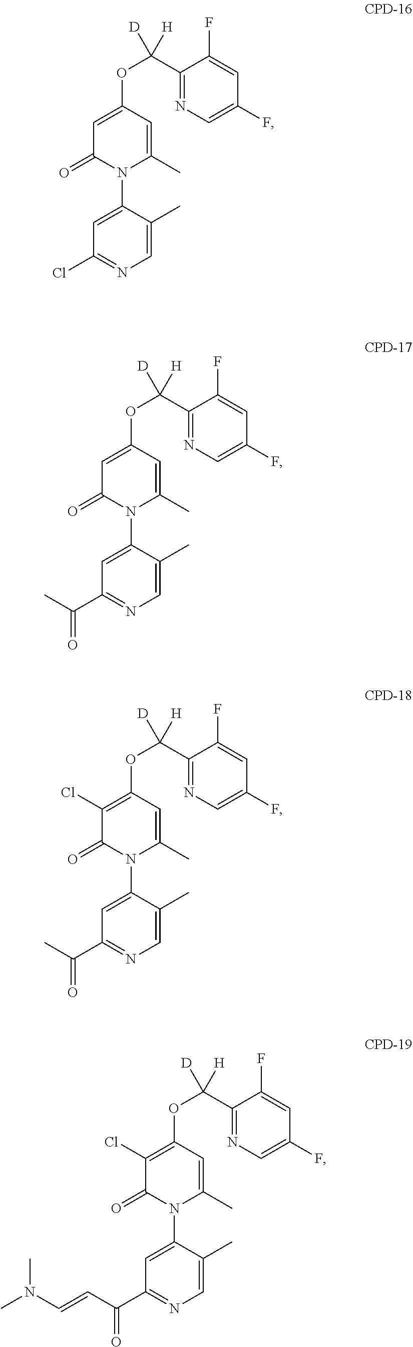 [SMILES: [2H]C(Oc1cc(C)n(-c2cc(Cl)ncc2C)c(=O)c1)c1ncc(F)cc1F ; [2H]C(Oc1cc(C)n(-c2cc(C(C)=O)ncc2C)c(=O)c1)c1ncc(F)cc1F ; [2H]C(Oc1cc(C)n(-c2cc(C(C)=O)ncc2C)c(=O)c1Cl)c1ncc(F)cc1F ; [2H]C(Oc1cc(C)n(-c2cc(C(=O)/C=C/N(C)C)ncc2C)c(=O)c1Cl)c1ncc(F)cc1F]

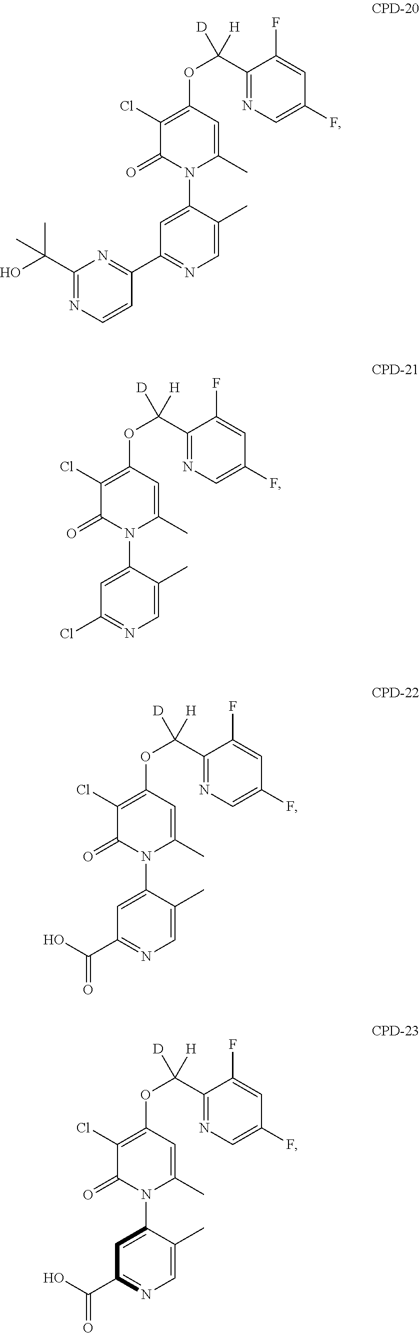

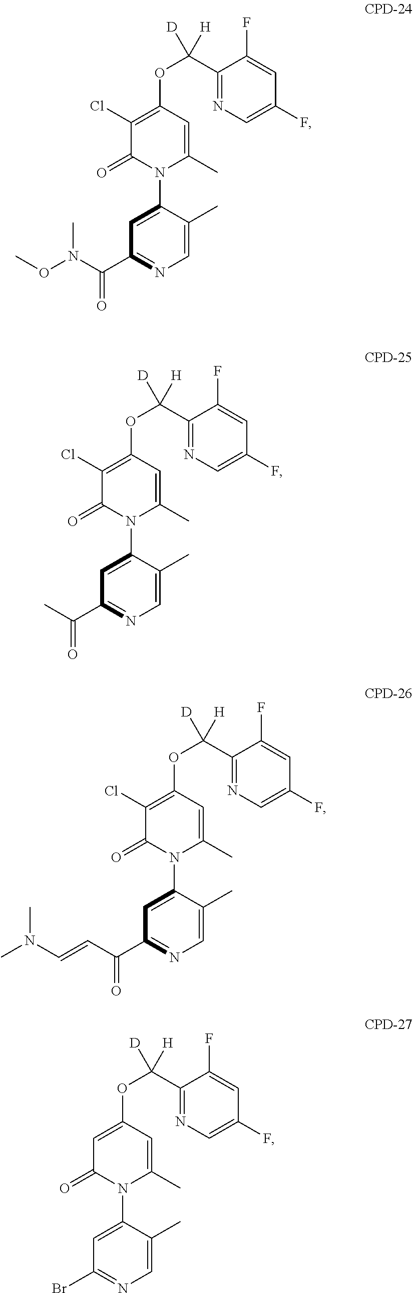

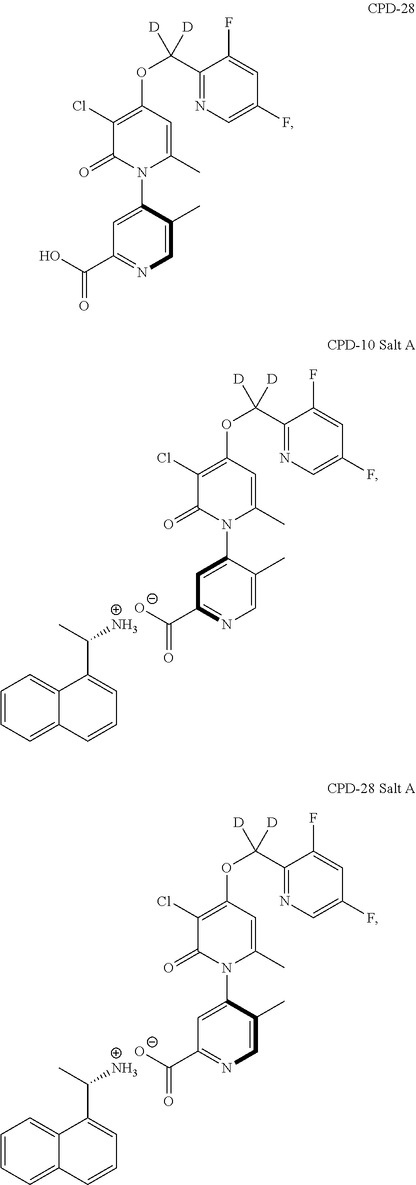

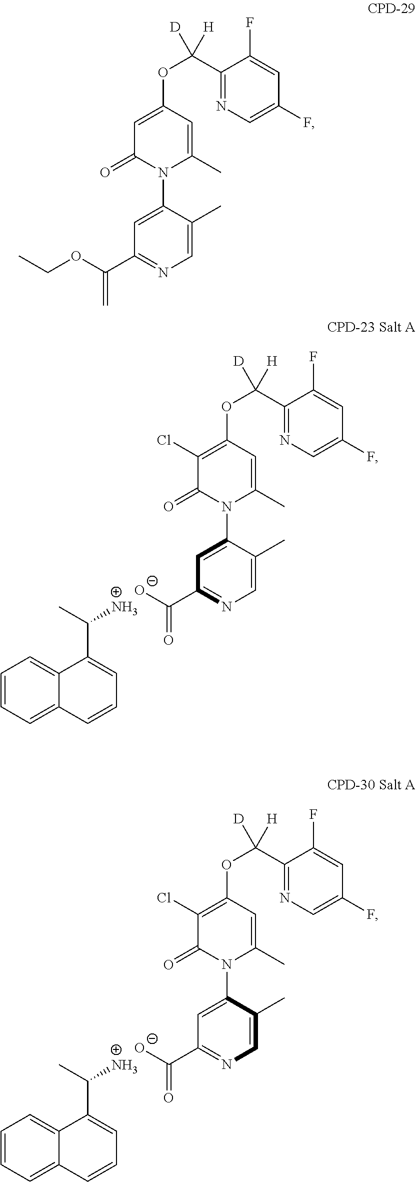

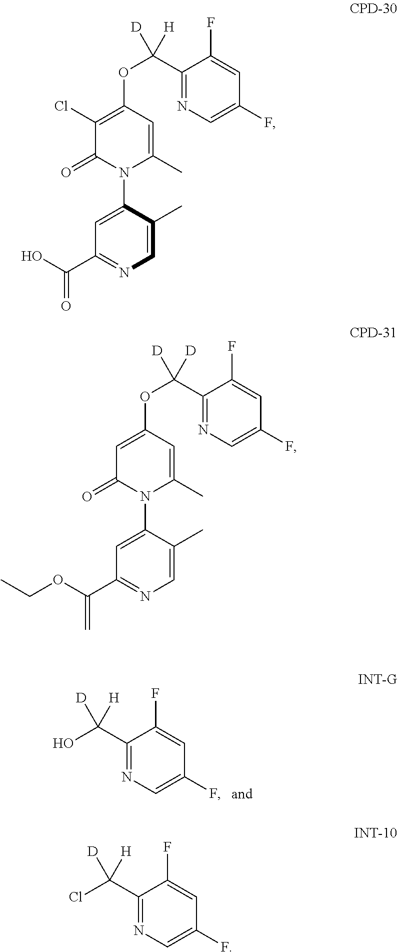

### Experimental Section

The compound of the present invention can, but are not limited to being prepared using the methods illustrated in the experimental procedures detailed below. The starting materials used to prepare the compounds of the present invention are commercially available or can be prepared using routine methods known in the art. Solvents and reagents, whose synthetic preparations are not described below, can be purchased at Sigma-Aldrich or Fisher Scientific.

Representative procedures for the preparation of compounds of this disclosure are outlined below.

### Example 1: Preparation of methyl 3,5-difluoropicolinate (INT-B)

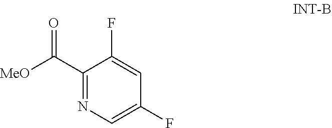 [SMILES: COC(=O)c1ncc(F)cc1F]

To a solution of 3,5-difluoropicolinic acid (INT-A) (2.48 kg, 15.58 mol) in methanol (7.44 L) at 0° C. was added thionyl chloride (791 mL, 0.7 eq, 0.32 vol). The solution was then warmed to 40° C. and stirred for 3 h. The reaction mixture was cooled to 25° C., and then concentrated in vacuo to remove volatiles. The residue was quenched with saturated sodium bicarbonate solution (15 L) and extracted three times (Total volume was 42.16 L) with ethyl acetate. The organic layer was washed with water and brine and then dried over anhydrous sodium sulfate. The sodium sulfate was filtered off and the solution was concentrated in vacuo to afford methyl 3,5-difluoropicolinate (INT-B) as white solid 2.3 kg (yield=85.5%) with 98.93% purity by HPLC. ¹H-NMR (400 MHz, DMSO-d₆): δ ppm 8.46 (1H, d, J=2.4 Hz), 7.37-7.33 (1H, m), 4.02 (3H, s). MS(ES) m/z 174.22 (M+H).

### Example 2: Preparation of 3,5-difluoropyridin-2-yl)methan-d2-ol (INT-C)

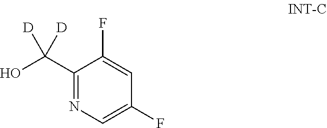 [SMILES: [2H]C([2H])(O)c1ncc(F)cc1F]

A stirred solution of THF (1.4 L, moisture content=0.038%) was cooled (ice-bath) to 25-30° C. Sodium borodeuteride (56.37 g, 80.86 mol, 99.9 atom % D) was added to the reaction mixture and stirred for 10-15 min. Zinc chloride (1.57 kg, 11.55 mol) was added to the reaction at 25-30° C. and stir for 5-10 min at 25-30° C. Cool the reaction mass to 15-20° C. and slowly add methyl 3,5-difluoropicolinate (INT-B) (Example 1, 20.00 kg, 115.52 mol) in THF (1.4 L, moisture content=0.038%) at 15-35° C. Cool the reaction mass to 25-30° C. and stir the reaction mass at 25-30° C. for 4-6 h. The progress of the reaction was monitored by HPLC. After completion, the reaction mass was quenched with water (5.0 vol.) at 20±5° C. The pH of the reaction mass was adjusted to ˜5.0-6.0 using 2N HCl (1.5 vol.) at 20±5° C., extracted with DCM (4×10 vol.) at 25-30° C. and the organic layer was dried over Na₂SO₄ (1.0 T) and washed with DCM (2.0 vol.) at 25-30° C. The DCM was distilled-off completely below 30° C. under vacuum to afford (3,5-difluoropyridin-2-yl)methan-d2-ol (INT-C) as colourless to light yellow colour liquid 16.70 kg (yield=98.00%) with 98.20% purity by HPLC, 98.40 D2-purity by LCMS. ¹H-NMR (400 MHz, DMSO-d₆): δ ppm 8.46 (1H, d, J=2 Hz), 7.93-7.88 (1H, m), 5.34 (3H, s); MS(ES) m/z 147.99 [M+H].

### Example 3: Preparation of 2-(chloromethyl-d2)-3,5-difluoropyridine (INT-01)

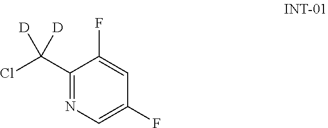 [SMILES: [2H]C([2H])(Cl)c1ncc(F)cc1F]

To a stirred solution at 0° C. of 3,5-difluoropyridin-2-yl)methan-d2-ol (INT-C) (Example 2, 16.60 kg, 112.83 mol) in dichloromethane (5 vol), was added thionyl chloride (10.6 L, 146.68 mol, 1.3 eq). The reaction mass was heated to 25-30° C. and maintained for 2-4 h. The reaction progress was monitored by HPLC. After completion the reaction was distilled to 1-2 vol. and co-distilled with DCM up to 1-2 vol. The reaction mass pH was adjusted to 7-8 with 10% aq. NaHCO₃ (5.0 vol) and extracted with DCM (4×5.0 vol) at 25-30° C. The organic layers were combined and washed with water (5.0 vol.) followed by 10% aq. NaCl (5.0 vol.) at 25-30° C. and dried over Na₂SO₄, concentrated in vacuo to afford 2-(chloromethyl-d2)-3,5-difluoropyridine (INT-01) as a colorless liquid 13.90 kg (yield=98.00%) with 98.82% purity by HPLC and 99.90% D2-purity by LCMS. 1H-NMR (400 MHz, DMSO-d₆): δ ppm 8.34 (1H, d, J=2 Hz), 7.27-7.22 (1H, m); MS(ES) m/z 166.02 (M+H).

### Example 4: Preparation of (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I)

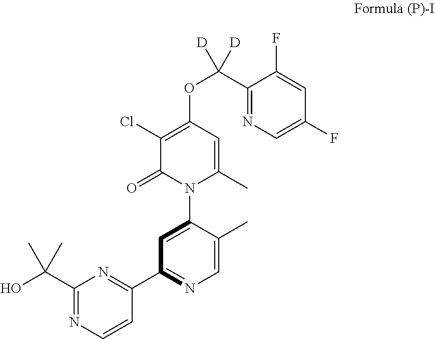

Step 1: Synthesis of 2′-bromo-4-((3,5-difluoropyridin-2-yl)methoxy-d2)-5′,6-dimethyl-2H-[1,4′-bipyridin]-2-one (CPD-15)

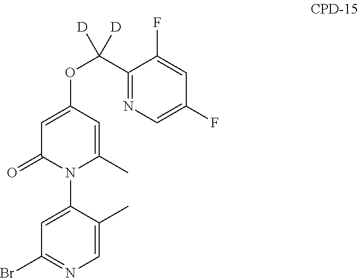 [SMILES: [2H]C([2H])(Oc1cc(C)n(-c2cc(Br)ncc2C)c(=O)c1)c1ncc(F)cc1F]

A solution of 2-(chloromethyl-d2)-3,5-difluoropyridine (INT-01) (Example 3, 454.4 g, 2.745 mol, 0.6 eq) in N,N-dimethylformamide (0.675 L) was added dropwise to a mechanically stirred suspension of 2′-bromo-4-hydroxy-5′,6-dimethyl-2H-[1,4′-bipyridin]-2-one (CPD-14) (1.35 kg, 4.576 mol, 1.0 eq) and potassium carbonate powder (948.67 g, 6.864 mol 1.5 eq) in N,N-dimethylformamide (5.4 L) at ambient temperature. The reaction mixture was stirred at ambient temperature for 12 hours. After 12 h 2-(chloromethyl-d2)-3,5-difluoropyridine (INT-01) (Example 3, 454.4 g, 2.745 mol, 0.6 eq) in N,N-dimethylformamide 0.675 L) was added drop-wise at ambient temperature. The reaction mixture was stirred at ambient temperature for another 12 hours, and the progress was monitored by HPLC/TLC. The reaction mass was then filtered, and the filtrate was poured into ice cold water (20.25 L). The mixture was stirred for 1 h, and the solid was filtered. The solid was washed with ethyl acetate (1.35 L). The ethyl acetate wash was added to the filtrates and stirred for 30 min. The organic layer was separated. The aqueous layer was twice extracted with ethyl acetate (13.5 L) and (13.5 L). The organic layers were combined, and then they were washed with water (2×13.5 L vol) and brine solution (2×6.75 L), and dried over sodium sulphate. The sodium sulfate was filtered, and the solution was concentrated to ˜2 L under vacuum at 45° C. MTBE (5.4 L) was added and the reaction mass was co-distilled to ˜2 L. MTBE (5.4 L) was added again and the solution was stirred for 8-9 h at ambient temperature. The reaction mass was filtered and washed with MTBE (1.35 L). The filter cake was dried well to afford 2′-bromo-4-((3,5-difluoropyridin-2-yl)methoxy-d2)-5′,6-dimethyl-2H-[1,4′-bipyridin]-2-one (CPD-15) as off-white solid 1.72 kg (yield=77%) with 95.77% purity by HPLC and 96.63% D2-purity by LCMS. ¹H-NMR (500 MHz, DMSO-d₆): δ ppm 8.59 (1H, d, J=2.5 Hz), 8.47 (1H, s), 8.09-8.05 (1H, m), 7.72 (1H, s), 6.13 (1H, s), 6.02 (1H, s), 1.96 (3H, s), 1.84 (3H, s). MS(ES) m/z 424.19 (M+H), 426.21 (M+3H).

Step 2: Synthesis of 2′-bromo-4-((3,5-difluoropyridin-2-yl)methoxy-d2)-5′,6-dimethyl-2H-[1,4′-bipyridin]-2-one (CPD-04)

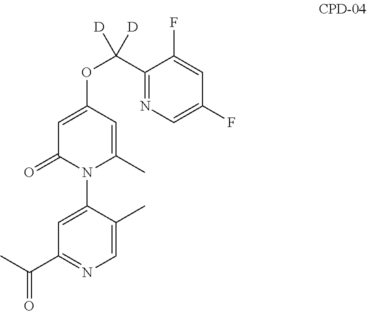 [SMILES: [2H]C([2H])(Oc1cc(C)n(-c2cc(C(C)=O)ncc2C)c(=O)c1)c1ncc(F)cc1F]

To a stirred suspension of 2′-bromo-4-((3,5-difluoropyridin-2-yl)methoxy-d2)-5′,6-dimethyl-2H-[1,4′-bipyridin]-2-one (CPD-15) (Example 4, Step 1, 1.1 kg, 2.594 mol, 1 eq) and 1,4-dioxane (8.25 L, 7.5 vol) in a 20 L RBF was added tributyl (1-ethoxy vinyl) tin (1.406 kg, 3.891 mol, 1.5 eq). The reaction mass was degassed with argon for 20 minutes. CuI (24.7 g, 0.1297 mol, 0.05 eq) was added, and the reaction mass was degassed with argon for an additional 30 minutes. Then Pd(dppf)Cl₂.DCM complex (84.87 g, 0.103 mol, 0.04 eq) was added and the reaction mass was degassed with argon for an additional 10 minutes. The reaction was heated to 100-105° C. and was stirred for 14 h. The reaction progress was monitored by HPLC which showed the desired product in 90.5 area %. The reaction mixture was cooled to room temperature, and activated carbon (220 g, 0.2 vol) and celite (110 g, 0.1 vol) were added. The mixture was stirred for 30 min at room temperature. The mixture was filtered through celite (550 g, 0.5 vol) and washed with 1,4-dioxane (2.2 L, 2 vol). The combined organic layers were concentrated under vacuum while heating from 55-60° C. to afford about 2.2 L of a solution. The solvent was exchanged by the addition of petroleum ether (2×2.2 L) and then distilling off the solvent. The reaction mass was at cooled to room temperature. Petroleum ether (20.9 L, 19 vol) was added and the mixture was stirred for 1-2 h at room temperature. The solid was filtered and washed with Pet-ether (2.2 L, 2 vol). The solid (1.09 kg wet) was taken in a round bottom flask. Water (5.5 L, 5 vol) was added and the mixture was cooled to 0-5° C. Concentrated HCl (1.1 L, 1 vol) was then added slowly keeping the temperature below 10° C. The cooling bath was removed and the mixture was allowed to warm to room temperature for 4-5 h. Activated carbon (550 g, 0.5 vol) was added into the reaction mass, and the mixture was stirred at room temperature for 1 h and then filtered through Celite. The Celite bed was washed with a 2 N HCl solution (3.3 L, 3 vol). The filtrate was cooled to 0-10° C. and basified with of aqueous sodium hydroxide solution (20%) to pH 9-10. It was extracted with MTBE (7×7.7 L). The combined organic layer was washed with 2M KF solution (2×2.2 L) and water (2×2.2 L). It was dried over Na₂SO₄. The Na₂SO₄ was filtered and washed with EtOAc (2.2 L, 2 vol). The filtrate was then concentrated under vacuum at 45-50° C. to afford 2.2 L of a solution. The solution was co-distilled sequentially with 30% of EtOAc/pet ether (6×2.2 L) and 20% of EtOAc/pet ether (2×2.2 L). Then 20% of EtOAc/pet ether (2.2 L, 2 vol) was charged and the mixture was stirred for 2-3 h at room temperature. Then solid was filtered and rinsed with 20% of EtOAc/pet ether (2.2 L, 2 vol). It was dried under vacuum to afford 2′-acetyl-4-((3,5-difluoropyridin-2-yl)methoxy-d2)-5′,6-dimethyl-2H-[1,4′-bipyridin]-2-one (CPD-04) (as off-white solid 616 g (yield=61.3%, crop-1) with 97.08% purity by HPLC and 96.4% D2-purity by LCMS). ¹H-NMR (500 MHz, DMSO-d₆): δ ppm 8.79 (1H, s), 8.59 (1H, d, J=2.4 Hz), 8.10-8.04 (1H, m), 7.78 (1H, s), 6.13 (1H, m), 6.04 (2H, d, J=2.8 Hz), 2.65 (3H, s), 2.09 (3H, s), 1.80 (3H, s); MS(ES) m/z 388.15 (M+H). A second crop afforded 2′-acetyl-4-((3,5-difluoropyridin-2-yl)methoxy-d2)-5′,6-dimethyl-2H-[1,4′-bipyridin]-2-one (CPD-04) as off-white solid 150 g (yield=15%, crop-2) with 97.54% purity by HPLC and 95.76% D2-purity by LCMS. ¹H-NMR (500 MHz, DMSO-d₆): δ ppm 8.79 (1H, s), 8.59 (1H, d, J=2 Hz), 8.09-8.05 (1H, m), 7.78 (1H, s), 6.13 (1H, m), 6.03 (2H, d, J=2.5 Hz), 2.65 (3H, s), 2.09 (3H, s), 1.80 (3H, s). MS(ES) m/z 388.15 (M+H).

Step 3: Preparation of 2′-acetyl-3-chloro-4-((3,5-difluoropyridin-2-yl)methoxy-d2)-5′,6-dimethyl-2H-[1,4′-bipyridin]-2-one (CPD-05)

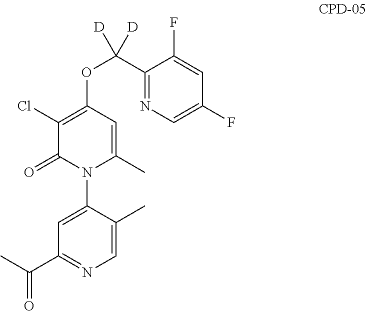 [SMILES: [2H]C([2H])(Oc1cc(C)n(-c2cc(C(C)=O)ncc2C)c(=O)c1Cl)c1ncc(F)cc1F]

To a stirred solution of 2′-acetyl-4-((3,5-difluoropyridin-2-yl)methoxy-d2)-5′,6-dimethyl-2H-[1,4′-bipyridin]-2-one (CPD-04) (Example 4, Step 2, 600 g, 1.548 mol, 1.0 eq) in IPA (9 L, 15 vol) was added N-chlorosuccinimide (248.17 g, 1.858 mol, 1.2 eq) portion wise at room temperature. Then reaction was heated to 65-70° C. and stirred for 2 h. A clear solution formed and after 1 h reaction a solid precipitated. Reaction progress was monitored by TLC/HPLC. When the reaction was complete, the reaction mixture was cooled to room temperature and stirred for 30-45 min. The solid was filtered, washed with IPA (50 ml) and dried under vacuum at 45° C. for 3 h to afford 2′-acetyl-3-chloro-4-((3,5-difluoropyridin-2-yl)methoxy-d2)-5′,6-dimethyl-2H-[1,4′-bipyridin]-2-one (CPD-05) as an off white solid. (470 g (yield=70%) from 3 batches 600 g, 160 g, and 238 g were mixed to afford 2′-acetyl-3-chloro-4-((3,5-difluoropyridin-2-yl)methoxy-d2)-5′,6-dimethyl-2H-[1,4′-bipyridin]-2-one (CPD-05) as an off white solid 770 g (yield=72%) with 96.4% purity by HPLC and 96.33% D2-purity by LCMS.) ¹H-NMR (400 MHz, DMSO-d₆): δ ppm 8.83 (1H, s), 8.83 (1H, s), 8.60 (1H, d, J=2.4 Hz), 8.11-8.07 (1H, m), 7.88 (1H, s), 6.79 (1H, s), 2.66 (1H, s), 2.09 (3H, s), 1.91 (3H, s); MS(ES) m/z 422.09 (M+H).

Step 4: Preparation of (E)-3-chloro-4-((3,5-difluoropyridin-2-yl)methoxy-d2)-2′-(3-(dimethylamino)acryloyl)-5′,6-dimethyl-2H-[1,4′-bipyridin]-2-one (CPD-06)

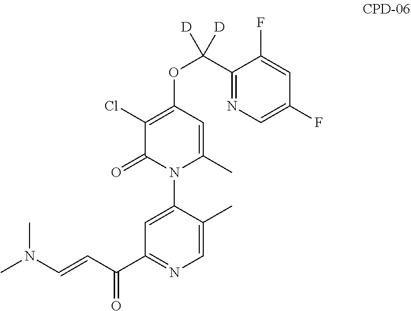 [SMILES: [2H]C([2H])(Oc1cc(C)n(-c2cc(C(=O)/C=C/N(C)C)ncc2C)c(=O)c1Cl)c1ncc(F)cc1F]

To a stirred solution of 2′-acetyl-3-chloro-4-((3,5-difluoropyridin-2-yl)methoxy-d2)-5′,6-dimethyl-2H-[1,4′-bipyridin]-2-one (CPD-05) (Example 4, Step 3, 450 g, 1.068 mol, 1.0 eq) in DMF (450 ml, 1 vol) was added DMF-DMA (382 g, 3.2 mol, 3.0 eq) at 25-30° C. Then reaction mass was heated to 75-80° C. and stirred for 16 h (Note—Initially a clear solution was observed and after 6 h a solid formed). The reaction progress was monitored by TLC/HPLC. After the reaction was complete, the mixture was cooled to room temperature and stirred for 30-45 min. The solid was filtered and washed with EtOAc (900 ml). After drying in the filter, the solid was dried under vacuum at 45° C. for 3 h to afford (E)-3-chloro-4-((3,5-difluoropyridin-2-yl)methoxy-d₂)-2′-(3-(dimethylamino)-acryloyl)-5′,6-dimethyl-2H-[1,4′-bipyridin]-2-one (CPD-06) as yellow solid (390 g (yield=76.7%). The product obtained from 2 batches 450 g, and 320 g were mixed to afford (E)-3-chloro-4-((3,5-difluoropyridin-2-yl)methoxy-d2)-2′-(3-(dimethylamino)acryloyl)-5′,6-dimethyl-2H-[1,4′-bipyridin]-2-one (CPD-06) as a yellow solid 635 g (yield=75.5%) with 95.04% purity by HPLC and 96.99% D2-purity by LCMS). ¹H-NMR (500 MHz, DMSO-d₆): δ ppm 8.71 (1H, s), 8.60 (1H, d, J=2.5 Hz), 8.11 (1H, m), 7.85 (1H, s), 7.82 (1H, d, J=5.5 Hz), 6.79 (1H, s), 6.38-6.30 (1H, d, J=12 Hz), 3.19 (3H, s), 2.94 (3H, s), 2.09 (3H, s), 1.91 (3H, s); MS(ES) m/z 477.20 (M+H).

Step 5: Preparation of 3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (CPD-07)

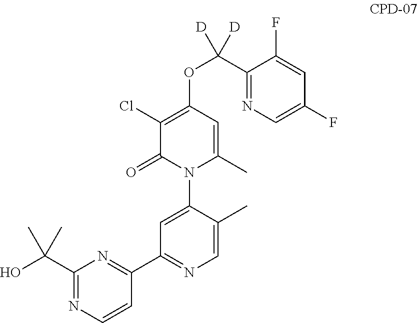 [SMILES: [2H]C([2H])(Oc1cc(C)n(-c2cc(-c3ccnc(C(C)(C)O)n3)ncc2C)c(=O)c1Cl)c1ncc(F)cc1F]

To a stirred solution of (E)-3-chloro-4-((3,5-difluoropyridin-2-yl)methoxy-d2)-2′-(3-(dimethylamino)acryloyl)-5′,6-dimethyl-2H-[1,4′-bipyridin]-2-one (CPD-06) (Example 4, Step 4, 400 g, 0.8389 mol, 1.0 eq) in DMF (2.4 L, 6 vol) was added potassium carbonate (289.86 g, 2.097 mol, 2.5 eq) portionwise at room temperature. The reaction mass was stirred for 30 min, and then 1-amino-2-hydroxy-2-methylpropan-1-iminium (INT-02) (348.82 g, 2.516 mol, 3.0 eq) was added portionwise at room temperature. The reaction mass was heated at 45-50° C. and was stirred for 12-14 h. The mixture became homogeneous, and the reaction progress was monitored by HPLC. After the reaction was complete, the mixture was cooled to 10-15° C. and stirred for 30 min. Water (4 L) was added and a solid precipitated. After stirring for an additional 1 h at 10-15° C., the solid was filtered, washed with water (4 L) and dried under vacuum. The wet solid was dissolved in DCM (4 L) and washed with water (2 L). The organic layer was dried over sodium sulphate. Sodium sulfate was filtered and activated charcoal was added to the DCM solution. The mixture was heated to 40° C. for 30 min. The mixture was filtered through a Celite bed and washed with DCM (2 L). The filtrate was distilled to about 800 ml under vacuum at 40° C. Methanol (2×800 ml) was added and then co-distilled to afford a solid. The reaction mass was cooled to room temperature. Methanol (3.2 L) was added, and the mixture was heated to 65-70° C. to afford clear solution. It was then cooled to 10-15° C., which caused a solid to precipitate, and then stirred for 1 h. The solid was filtered, washed with methanol (800 ml), dried under vacuum to afford 3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (CPD-07) crop −1 as an off-white solid 321 g (yield=74%) and 3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (CPD-07) crop-2 as an off-white solid 30 g (yield=6.9%) with 99.22% purity by HPLC and 95.14% D2-purity by LCMS). ¹H-NMR (400 MHz, DMSO-d₆): δ ppm 8.98 (1H, d, J=8 Hz), 8.86 (1H, s), 8.69 (1H, s), 8.61 (1H, d, J=4 Hz), 8.25 (1H, d, J=8 Hz), 8.13-8.07 (1H, m), 6.83 (1H, d, J=12 Hz), 2.11 (3H, s), 2.00 (3H, s), 1.55 (6H, s); MS(ES) m/z 516.45 (M+H).

Step 6: Preparation of (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I) and (M)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (M)-I)

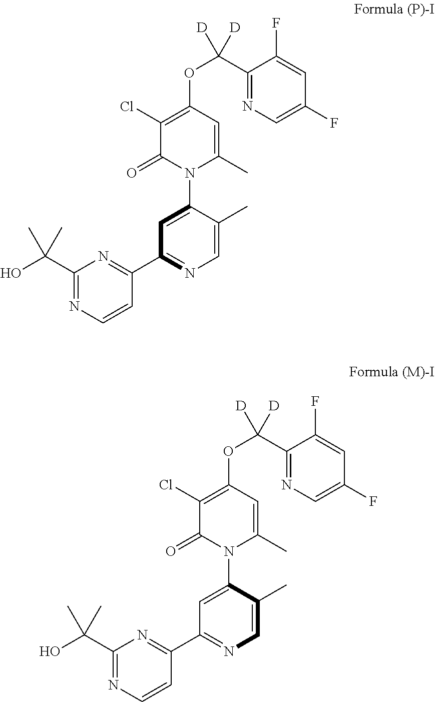

### Preparative SFC Conditions

Column/Dimensions: Chiralcel OD-H (200×25×5μ)
% CO₂: 50%
% Co solvent: 50% (100% Isopropanol)
Total Flow: 90 g/min
Back Pressure: 100.0 bar
Temperature: 30.0° C.
UV: 214.0 nm
Stack time: 10.0 min
Load/Inj: 720.63 mg/injection
   SFC Purification: (Lot-1: 321 g), (Lot-2: 160.9 g), (Lot-3: 81.7 g), (Lot-4: 89.8 g), (Lot-5 :219 g-Obtained from Peak-2 Racemization) 5 lots (872.4 g) of CPD-07 was submitted for SFC purification (conditions described in attached file), to afford Formula (P)-I (Peak-1)=351 g, Formula (M)-I (Peak-2)=340.1 g, Mixture=82.1 g.

Step 7: Preparation (Crystallization) of (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I)

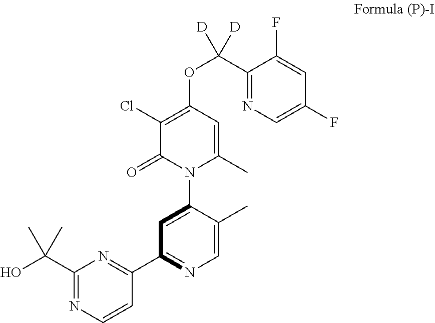

A suspension of (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I) (Example 10, Step 1, 137 g, 0.265 mol, 1 eq.) in isopropyl alcohol (2.74 L, 20 vol) was heated to 65-70° C. and stirred for 1.0 h (a clear solution was observed). The reaction mass was filtered hot at 65-70° C. The filtrate was cooled to room temperature and stirred for 16 h. The solid was filtered and washed with isopropyl alcohol (137 ml, 1 vol). The solid was dried under vacuum at 40° C. to afford (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I) as an off white solid 115.5 g (yield=84.3%) with 99.81% purity by HPLC and 95.46% D2-purity by LCMS). ¹H-NMR (400 MHz, DMSO-d₆): δ ppm 8.97 (1H, d, J=5.2 Hz), 8.85 (1H, s), 8.68 (1H, s), 8.61 (1H, d, J=2.4 Hz), 8.24 (1H, d, J=5.2 Hz), 8.12-8.07 (1H, m), 6.84 (1H, s), 5.24 (1H, s), 2.10 (3H, s), 1.98 (3H, s), 1.53 (3H, s), 1.52 (3H, s); MS(ES) m/z 516.26 (M+H).

Step 8: Preparation of 3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (CPD-07)

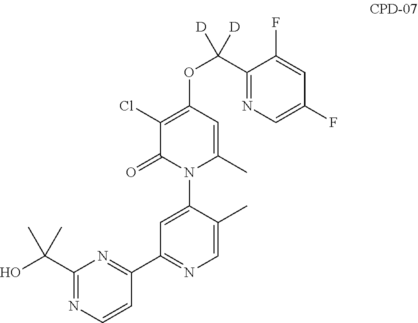 [SMILES: [2H]C([2H])(Oc1cc(C)n(-c2cc(-c3ccnc(C(C)(C)O)n3)ncc2C)c(=O)c1Cl)c1ncc(F)cc1F]

A stirred solution of (M)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (M)-I) (Example 9 Step 1, 90 g, 174.26 mmol, 1 eq.) in toluene (900 mL, 10 vol) was heated to 120° C. for 4 h. The progress of the reaction was monitored by chiral HPLC. After completion of the reaction, the solution was cooled to room temperature and was stirred for 30 min. The solid was filtered and washed with toluene (180 ml and dried under vacuum at 45° C. to afford racemic 3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (CPD-07) as off white solid 87 g (yield=96.6%). Another two batches of 64 g and 68 g of CPD-07 were blended with the above batch and re-submitted for SFC Purification (see Example 4 Step 6) to afford (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I) as an off white solid 219 g (yield=99.5%) with 98.77% purity by HPLC, (Isomer I:Isomer II) 49.38%: 50.62% and 99.43% D2-purity by LCMS. ¹H-NMR (400 MHz, DMSO-d₆): δ ppm 8.97 (1H, d, J=5.2 Hz), 8.86 (1H, s), 8.69 (1H, s), 8.61 (1H, d, J=2.5 Hz), 8.24 (1H, d, J=5.2 Hz), 8.12-8.07 (1H, m), 6.83 (1H, s), 5.24 (1H, s), 2.10 (3H, s), 1.98 (3H, s), 1.53 (3H, s), 1.52 (3H, s); MS(ES) m/z 516.38 (M+H).

### Example 5: Preparation of 2′-chloro-4-((3,5-difluoropyridin-2-yl)methoxy-d2)-5′,6-dimethyl-2H-[1,4′-bipyridin]-2-one (CPD-03)

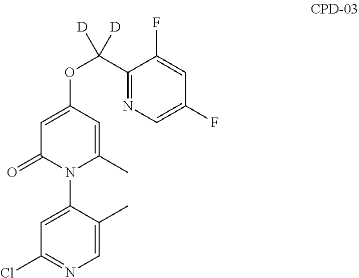 [SMILES: [2H]C([2H])(Oc1cc(C)n(-c2cc(Cl)ncc2C)c(=O)c1)c1ncc(F)cc1F]

To a stirred suspension of 2′-chloro-4-hydroxy-5′,6-dimethyl-2H-[1,4′-bipyridin]-2-one (CPD-02) (80 g, 31.9 mol, 1.0 eq) in DMF (400 mL, 5 vol), was added K₂CO₃ (66.6 g, 48.3 mol 1.5 eq) to the reaction mass at 25-30° C. and Stirred for 30 minutes at 25-35° C. Then added a solution of 2-(chloromethyl-d2)-3,5-difluoropyridine (INT-01) (63.5 g, 38.4 mol, 1.2 eq) in DMF (1 vol.) to the reaction mass at 25-35° C. The reaction mixture was stirred at rt for another 36 hours. The progress of the reaction was monitored by TLC and HPLC. After completion of the reaction, charged water (600 ml, 7.5 Vol.) and stirred for 15-20 minutes. The reaction mass was extracted with ethyl acetate (3×600 ml), combined ethyl acetate layer was washed with brine (2×400 ml). Distilled ethyl acetate layer up to 1-2 vol. and co-distilled with MTBE (2×240 ml). Charged MTBE (320 ml, 4 vol) and stirred for 4 h, filtered the solid and dried under vacuum at below 45° C. to afford crude 2′-chloro-4-((3,5-difluoropyridin-2-yl)methoxy-d2)-5′,6-dimethyl-2H-[1,4′-bipyridin]-2-one (CPD-03) as off-white solid 85.0 g (yield=68.3%) with 95.6% HPLC purity and 96.67% D2-purity by LCMS. Above compound (1.5 g) was purified by reverse phase chromatography to afford pure 2′-chloro-4-((3,5-difluoropyridin-2-yl)methoxy-d2)-5′,6-dimethyl-2H-[1,4′-bipyridin]-2-one (CPD-XXX) as off-white solid 1.1 g (yield=68.3%) with 99.14% purity by and 96.66% D2-purity by LCMS. ¹H-NMR (400 MHz, DMSO-d₆): δ ppm 8.49 (1H, s), 8.09-8.04 (1H, m), 7.60 (1H, s), 6.13 (1H, m), 6.03 (1H, d, J=2.8 Hz), 1.98 (3H, s), 1.84 (3H, s). MS(ES) m/z 380.10 (M+H).

### Example 6: Preparation of 2′-acetyl-4-((3,5-difluoropyridin-2-yl)methoxy-d2)-5′,6-dimethyl-2H-[1,4′-bipyridin]-2-one (CPD-04)

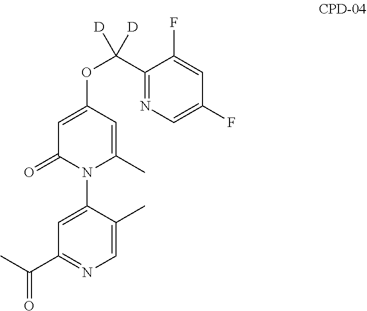 [SMILES: [2H]C([2H])(Oc1cc(C)n(-c2cc(C(C)=O)ncc2C)c(=O)c1)c1ncc(F)cc1F]

To a stirred suspension of 2′-chloro-4-((3,5-difluoropyridin-2-yl)methoxy-d2)-5′,6-dimethyl-2H-[1,4′-bipyridin]-2-one (CPD-03) (Example 5, 500 mg, 1.31 mol, 1.0 eq) and 1,4-dioxane (4.0 mL, 8 vol) in a 100 mL RBF was added tributyl (1-ethoxy vinyl) tin (715 mg, 1.97 mol, 1.5 eq). The reaction mass was degassed with argon for 20 minutes. CuI (2.24 g, 0.065 mol, 0.065 eq) was added, and the reaction mass was degassed with argon for an additional 30 minutes. Then Pd(dppf)Cl₂.DCM complex (43 mg, 0.052 mol, 0.04 eq) was added and the reaction mass was degassed with argon for an additional 10 minutes. The reaction was heated to 100-105° C. and was stirred for 16 h. The reaction progress was monitored by HPLC which showed the desired product in 85.7 area %. The reaction mixture was cooled to room temperature, filtered through celite, the filtrated was added to a clean dry RBF and concentrated HCl (0.5 mL) was then added slowly keeping the temperature below 10° C. The cooling bath was removed and the mixture was allowed to warm to room temperature for 1 h then basified with aqueous sodium hydroxide solution (20%) to pH 9-10 and extracted with MTBE (2×50 mL). The combined organic layer was washed with 2M KF solution (2×10 mL) and water (2×25 mL). It was dried over Na₂SO₄. The Na₂SO₄ was filtered and the filtrate was then concentrated under vacuum at 45-50° C. to afford crude (320 mg) 2′-acetyl-4-((3,5-difluoropyridin-2-yl)methoxy-d2)-5′,6-dimethyl-2H-[1,4′-bipyridin]-2-one (CPD-04) which was further purified by FCC on silica gel. Desired fractions were collected combined, freed from solvent under vacuum to afford crude (240 mg) 2′-acetyl-4-((3,5-difluoropyridin-2-yl)methoxy-d2)-5′,6-dimethyl-2H-[1,4′-bipyridin]-2-one (CPD-04). This material was suspended in 30% ethyl acetate/pet ether, stirred for 1 h at rt, filtered to afford 2′-acetyl-4-((3,5-difluoropyridin-2-yl)methoxy-d2)-5′,6-dimethyl-2H-[1,4′-bipyridin]-2-one (CPD-04) as an off-white solid (150 mg (yield=29.4%) with 96.85% HPLC purity and 96.54% D2-purity). ¹H-NMR (400 MHz, DMSO-d₆): δ ppm 8.79 (1H, s), 8.59 (1H, d, J=2 Hz), 8.10-8.04 (1H, m), 7.78 (1H, s), 6.13 (1H, d, J=1.6 Hz), 6.03 (2H, d, J=2.4 Hz), 2.65 (3H, s), 2.09 (3H, s), 1.80 (3H, s). MS(ES) m/z 388.23 (M+H).

### Example 7: Preparation of (P)-2′-chloro-4-((3,5-difluoropyridin-2-yl)methoxy-d2)-5′,6-dimethyl-2H-[1,4′-bipyridin]-2-one (Formula (P)-I) via chiral separation with (S)-2-naphthylethyl amine

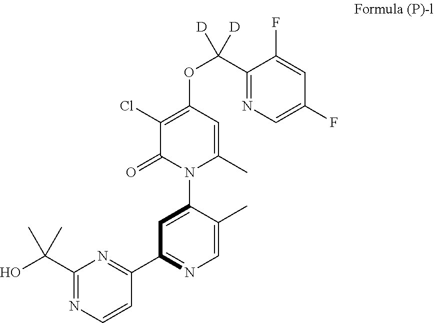

Step 1: Preparation of 2′-chloro-4-((3,5-difluoropyridin-2-yl)methoxy-d2)-5′,6-dimethyl-2H-[1,4′-bipyridin]-2-one (CPD-03)

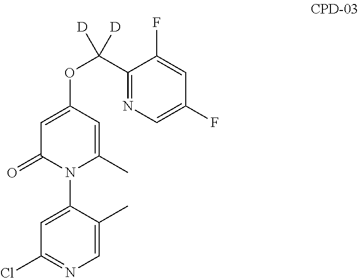 [SMILES: [2H]C([2H])(Oc1cc(C)n(-c2cc(Cl)ncc2C)c(=O)c1)c1ncc(F)cc1F]

To a stirred suspension of 2′-chloro-4-hydroxy-5′,6-dimethyl-2H-[1,4′-bipyridin]-2-one (CPD-02) (80 g, 31.9 mol, 1.0 eq) in DMF (5 vol), was added K₂CO₃ (66.6 g, 48.3 mol 1.5 eq) at 25-30° C. and stirred for 30 minutes. Then added a solution of 2-(chloromethyl-d2)-3,5-difluoropyridine (INT-01) (63.5 g, 38.4 mol, 1.2 eq) in DMF (1 vol.) at 25-35° C. The reaction mixture was stirred for another 36 hours. The progress of the reaction was monitored by TLC/HPLC. After completion of the reaction, charged water (600 ml, 7.5 vol) and stirred for 15-20 min. Reaction mass was extracted with ethyl acetate (3×6 00 ml). Combined ethyl acetate layer was washed with brine (2×400 ml). Ethyl acetate was distilled under vacuum (˜1-2 vol) and co-distilled with MTBE (2×240 ml). Charged MTBE (320 ml, 4 vol) and stirred for ˜4 h. Filtered the solid and dried under vacuum at 45° C. to afford crude 2′-chloro-4-((3,5-difluoropyridin-2-yl)methoxy-d2)-5′,6-dimethyl-2H-[1,4′-bipyridin]-2-one (CPD-03) (85 g, 70.1% yield) as off-white solid. 1.5 g of crude 2′-chloro-4-((3,5-difluoropyridin-2-yl)methoxy-d2)-5′,6-dimethyl-2H-[1,4′-bipyridin]-2-one (CPD-03) was further purified by reverse phase chromatography to afford 2′-chloro-4-((3,5-difluoropyridin-2-yl)methoxy-d2)-5′,6-dimethyl-2H-[1,4′-bipyridin]-2-one (CPD-03) as an off-white solid 1.1 g (yield=67%) with 99.14% purity by HPLC and 96.66% D2-purity by LCMS. 1H-NMR (500 MHz, DMSO-d₆) δ ppm: 8.59 (1H, d, J=2.4 Hz), 8.49 (1H, s), 8.09-8.04 (1H, m), 7.60 (1H, s), 6.13 (1H, m), 6.03 (1H, d, J=2.8 Hz), 1.98 (3H, s), 1.84 (3H, s). MS(ES) m/z 380.10 (M+H).

Step 2: Preparation of 2′,3-dichloro-4-((3,5-difluoropyridin-2-yl)methoxy-d2)-5′,6-dimethyl-2H-[1,4′-bipyridin]-2-one (CPD-08)

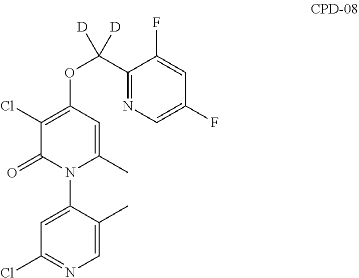 [SMILES: [2H]C([2H])(Oc1cc(C)n(-c2cc(Cl)ncc2C)c(=O)c1Cl)c1ncc(F)cc1F]

To a stirred solution of 2′-chloro-4-((3,5-difluoropyridin-2-yl)methoxy-d2)-5′,6-dimethyl-2H-[1,4′-bipyridin]-2-one (CPD-03) (Example 7, Step 1, 75 g, 197 mmol, 1.0 eq) in IPA (1125 ml, 15 vol), N-chlorosuccinimide (31.6 g, 236 mmol, 1.2 eq) was added portion wise at RT. The reaction was heated to 65-70° C. and maintained for 2 h. Clear solution was formed and after 1 h solid formation was observed. Reaction progress was monitored by TLC and after completion the reaction mixture was cooled to RT and stirred for 30-45 min., filtered, washed with IPA (75 ml) and dried under vacuum at 45° C. for 3 h to afford 2′,3-dichloro-4-((3,5-difluoropyridin-2-yl)methoxy-d2)-5′,6-dimethyl-2H-[1,4′-bipyridin]-2-one (CPD-08) as an off-white solid 63 g (yield=77%) with 96.88% HPLC purity and 96.74% D2-purity by LCMS. 1H-NMR (400 MHz, DMSO-d₆) δ ppm: 8.60 (1H, d, J=2 Hz), 8.53 (1H, s), 8.11-8.06 (1H, m), 7.68 (1H, s), 6.79 (1H, s), 1.97 (3H, s), 1.95 (3H, s). MS(ES) m/z 414.12 (M+H).

Step 3: Preparation of 3-chloro-4-((3,5-difluoropyridin-2-yl)methoxy-d2)-5′,6-dimethyl-2-oxo-2H-[1,4′-bipyridine]-2′-carboxylic acid (CPD-09)

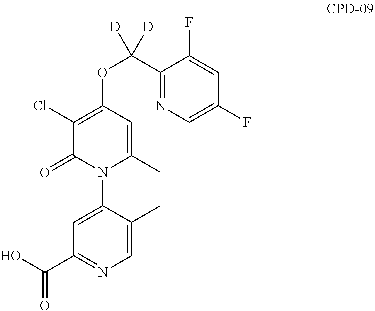 [SMILES: [2H]C([2H])(Oc1cc(C)n(-c2cc(C(=O)O)ncc2C)c(=O)c1Cl)c1ncc(F)cc1F]

A clean and dried 2 L autoclave flask was de-poisoned by stirring a mixture of 2′,3-dichloro-4-((3,5-difluoropyridin-2-yl)methoxy-d2)-5′,6-dimethyl-2H-[1,4′-bipyridin]-2-one (CPD-08) (Example 7, Step 2, 500 mg), Pd(dppf)Cl₂ (500 mg), in acetonitrile (250 ml) and water (250 ml) and CO pressure 40-50 psi for about 1 h at 75° C. The vessel was emptied and rinsed with acetonitrile. The rinse was discarded. 2′,3-dichloro-4-((3,5-difluoropyridin-2-yl)methoxy-d2)-5′,6-dimethyl-2H-[1,4′-bipyridin]-2-one (CPD-08) (Example 7, Step 2, 62 g, 149 mmol, 1.0 eq), acetonitrile (496 ml, 8.0 vol.) and water (248 ml, 4.0 vol.) were charged in the autoclave at rt. The mixture was purged with argon gas for 30 minutes and K₂CO₃ (22.10 g, 298 mmol, 3.0 eq.) was added followed by Pd(dppf)Cl₂ (6.1 g, 7.48 mmol, 0.05 eq). The reaction mass was further purged with argon for 15 minutes. The reaction vessel was closed and pressurised with CO (40-45 psi) and stirred for a minute. Pressure was released and again applied CO pressure 100 psi (5.0 kg). The reaction mixture was mechanically stirred and heated at 75° C. for 36 h. The progress of the reaction was monitored by TLC/HPLC. After completion the reaction mixture was cooled to rt and collected from autoclave. cooled to 25-30° C. and pressure was released. Reaction mixture was de-gassed with argon. Unloaded the reaction mass and added water (310 ml, 5 vol). pH was adjusted to 14 with 2N NaOH solution. Then reaction mass was washed with MTBE (3×50 ml). Aqueous layer was filtered through hyflow bed. The pH of filtered mL's was adjusted to −1-2 with 6 N HCl and stirred for ˜2 h at 25-30° C. The precipitated solid was filtered. It was washed with water (620 ml, 10 vol.) followed by ethanol (162 ml, 1 vol.) and dried under vacuum to afford 3-chloro-4-((3,5-difluoropyridin-2-yl)methoxy-d2)-5′,6-dimethyl-2-oxo-2H-[1,4′-bipyridine]-2′-carboxylic acid (CPD-09) as an off-white solid 56.5 g (yield=89%) with 99.01% purity by HPLC and 96.21% D2-purity by LCMS. ¹H-NMR (400 MHz, DMSO-d₆) δ ppm: 13.36 (1H, s), 8.80 (1H, s), 8.60 (1H, s), 8.12-8.06 (1H, m), 7.92 (1H, s), 6.79 (1H, s), 2.07 (3H, s), 1.92 (3H, s). MS(ES) m/z 424.14 (M+H).

Step 4: Preparation of (P)-3-chloro-4-((3,5-difluoropyridin-2-yl)methoxy-d2)-5′,6-dimethyl-2-oxo-2H-[1,4′-bipyridine]-2′-carboxylate (5)-1-(naphthalen-1-yl)ethan-1-aminium (Isomer I-salt) (CPD-10 Salt A) and (M)-3-chloro-4-((3,5-difluoropyridin-2-yl)methoxy-d2)-5′,6-dimethyl-2-oxo-2H-[1,4′-bipyridine]-2′-carboxylate (5)-1-(naphthalen-1-yl)ethan-1-aminium (Isomer II-salt) (CPD-28 Salt A)

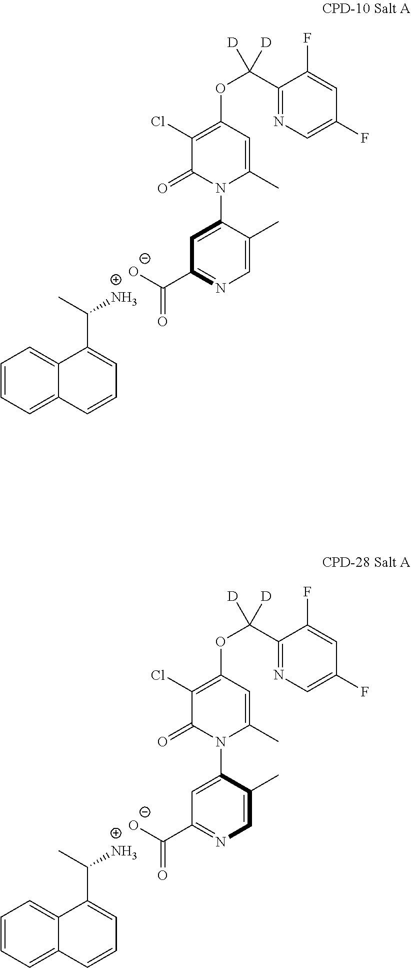

A stirred suspension of 2′-acetyl-3-chloro-4-((3,5-difluoropyridin-2-yl)methoxy-d2)-5′,6-dimethyl-2H-[1,4′-bipyridin]-2-one (CPD-09) (Example 7, Step 3, 30 g, 70.92 mmol, 1.0 eq.) in 5% of DMSO: anisole (210 mL, 7 vol) was slowly heated to 110-115° C. The reaction mass was stirred for 10-15 min. A solution of (S)-2-naphthylethyl amine (12.1 g, 70.92 mmol, 1.04 eq.) in 5% of DMSO: anisole (420 mL, 14 vol.) was slowly added to the reaction mass over 30 min. The reaction was stirred for 72 h at 110-115° C., Reaction was monitored by Chiral HPLC every 24 h. The above reaction mixture was allowed to cool to RT. The solid was filtered and washed with pet-ether (10 vol×3), dried under vacuum for 4 h at 40° C. to afford Preparation of (P)-3-chloro-4-((3,5-difluoropyridin-2-yl)methoxy-d2)-5′,6-dimethyl-2-oxo-2H-[1,4′-bipyridine]-2′-carboxylate (S)-1-(naphthalen-1-yl)ethan-1-aminium (Isomer I-salt) (CPD-10 Salt A) and (M)-3-chloro-4-((3,5-difluoropyridin-2-yl)methoxy-d2)-5′,6-dimethyl-2-oxo-2H-[1,4′-bipyridine]-2′-carboxylate (S)-1-(naphthalen-1-yl)ethan-1-aminium (Isomer II-salt) (CPD-28 Salt A) total of both (S)-2-naphthylethyl amine salts 36 g (yield=85.49%) with 98.25% purity by HPLC, (Isomer I:Isomer II) 97.76%: 2.24% (95.52% ee) chiral purity by chiral HPLC.

Step 5: Preparation of (P)-3-chloro-4-((3,5-difluoropyridin-2-yl)methoxy-d2)-5′,6-dimethyl-2-oxo-2H-[1,4′-bipyridine]-2′-carboxylic acid (CPD-10)

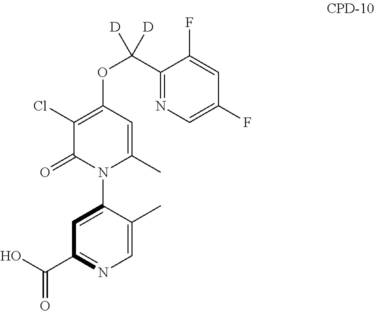

The mixture of salts (P)-3-chloro-4-((3,5-difluoropyridin-2-yl)methoxy-d2)-5′,6-dimethyl-2-oxo-2H-[1,4′-bipyridine]-2′-carboxylate (S)-1-(naphthalen-1-yl)ethan-1-aminium (Isomer I-salt) (CPD-10 Salt A) and (M)-3-chloro-4-((3,5-difluoropyridin-2-yl)methoxy-d2)-5′,6-dimethyl-2-oxo-2H-[1,4′-bipyridine]-2′-carboxylate (S)-1-(naphthalen-1-yl)ethan-1-aminium (Isomer II-salt) (CPD-28 Salt A) (Example 7, Step 4, 36 g, 60.6 mmol, (Isomer I:Isomer II) 97.76%: 2.24% (95.52% ee) was dissolved in water (1080 ml) and basified with 2N NaOH (108 ml, 3 vol) to pH 14 and extracted with MTBE (3×360 ml). Aqueous layer was filtered through celite, washed with MTBE (180 ml, 5 vol). It was acidified with 6N HCl (90 ml, 2.5 vol) pH to −2 and stirred for 2 h. Precipitated solid was filtered, washed with water (10 vol) and dried under vacuum to afford (P)-3-chloro-4-((3,5-difluoropyridin-2-yl)methoxy-d2)-5′,6-dim ethyl-2-oxo-2H-[1,4′-bipyridine]-2′-carb oxylic acid (CPD-10) as off-white solid 24 g (yield=93.9%) with 98.77 HPLC purity, (Isomer I:Isomer II) 99.94%:0.06% (99.88% ee) chiral purity by chiral HPLC, and 96.25% D2-purity by LCMS. 1-H-NAIR (400 MHz, DMSO-d₆) δ ppm: 13.35 (1H, s), 8.80 (1H, s), 8.60 (1H, s), 8.12-8.06 (1H, m), 7.97 (1H, s), 6.79 (1H, s), 2.07 (3H, s), 1.92 (3H, s). MS(ES) m/z 424.18 (M+H).

Step 6: Preparation of (P)-3-chloro-4-((3,5-difluoropyridin-2-yl)methoxy-d2)-N-methoxy-N,5′,6-trimethyl-2-oxo-2H-[1,4′-bipyridine]-2′-carboxamide (CPD-11)

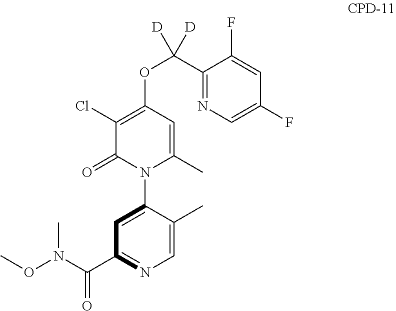

To a stirred solution of (P)-3-chloro-4-((3,5-difluoropyridin-2-yl)methoxy-d2)-5′,6-dimethyl-2-oxo-2H-[1,4′-bipyridine]-2′-carboxylic acid (CPD-10) (Example 7, Step 5, 20 g, 47.2 mmol, (Isomer I:Isomer II) 99.94%:0.06% (99.88% ee) in DCM (160 ml, 8.0 vol.) at 0-5° C. EDC.HCl (9.97 g, 52 mmol, 1.1 eq.) was added to the reaction mass under argon atmosphere and stirred for 15 min. Then added N, O-dimethyl hydroxylamine hydrochloride (6.9 g, 70.8 mmol, 1.5 eq.) at 0-5° C. and stirred for 15 min. TEA (8.2 ml, 59 mmol, 1.25 eq.) was added drop-wise to the reaction mass and stirred for 2 h. Progress of the reaction was monitored by TLC and HPLC. After completion of reaction the reaction mass was quenched with water (200 ml, 10 vol.). It was allowed to warm up to RT ˜25° C. and stirred for 10 minutes. The two layers were separated, and aqueous layer was extracted with DCM (2×100 ml, 5 vol.). Combined DCM layer was washed with water (2×60 ml), dried and concentrated under vacuum. The crude was co-distilled with MTBE (3×40 ml) completely. Then fourth time crude solid was suspended in MTBE (40 ml, 2 vol.) and stirred for 2 h. Filtered the solid, washed with MTBE (20 ml, 1 vol.) and dried under vacuum to afford (P)-3-chloro-4-((3,5-difluoropyridin-2-yl)methoxy-d2)-N-methoxy-N,5′,6-trimethyl-2-oxo-2H-[1,4′-bipyridine]-2′-carboxamide (CPD-11) as an off white solid 17 g (yield=77%) with 98.76 purity by HPLC, (Isomer I:Isomer II) 98.71%:2.19% (95.62% ee) chiral purity by chiral HPLC, 96.21% D2-purity by LCMS. ¹H-NMR (400 MHz, DMSO-d₆) δ ppm: 8.70 (1H, s), 8.60 (1H, d, J=2.4 Hz), 8.11-8.06 (1H, m), 7.62 (1H, s), 6.79 (1H, s), 3.67 (3H, s), 3.29 (3H, s), 2.04 (3H, s), 1.93 (3H, s). MS(ES) m/z 467.22 (M+H).

Step 7: Preparation of (P)-2′-acetyl-3-chloro-4-((3,5-difluoropyridin-2-yl)methoxy-d₂)-5′,6-dimethyl-2H-[1,4′-bipyridin]-2-one (CPD-12)

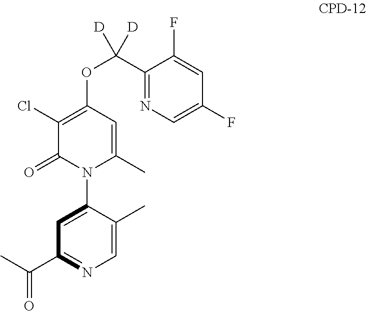

A stirred suspension of (P)-3-chloro-4-((3,5-difluoropyridin-2-yl)methoxy-d2)-N-methoxy-N,5′,6-trimethyl-2-oxo-2H-[1,4′-bipyridine]-2′-carboxamide (CPD-11) (Example 7, Step 6, 14 g, 29.98 mmol, 1.0 eq., (Isomer I:Isomer II) 98.71%: 2.19% (95.62% ee) in THF (200 ml, 10 vol.) was cooled to −10° C. Methyl magnesium bromide solution (2M in THF, 22.4 ml, 44.9 mmol, 1.5 eq.) was added to the reaction mass under argon atmosphere and stirred at −10° C. to 0° C. for 2 h. The progress of the reaction was monitored by TLC and HPLC and upon completion the reaction was quenched with saturated NH4Cl (280 ml, 20 vol), allowed to −25° C. (— 1 h). It was extracted with EtOAc (140 ml×3). Combined EtOAc layer was washed with water (2×70 ml) and brine solution. EtOAc layer were completely distilled, and the residue was co-distilled with MeOH (2×28 ml). Charged MeOH (70 ml, 5 vol.) and stirred for 1 h at 60° C. and allow to rt. Solid was filtered, washed with MeOH (14 ml, 1 vol). It was and dried under vacuum below 40° C., to afford (P)-2′-acetyl-3-chloro-4-((3,5-difluoropyridin-2-yl)methoxy-d2)-5′,6-dimethyl-2H-[1,4′-bipyridin]-2-one (CPD-12) as an off white solid, 10 g (yield=78.7%) with 99.13% purity by HPLC, (Isomer I:Isomer II) 98.79%: 1.21% (97.58% ee) chiral purity by chiral HPLC and 96.60% D2-purity by LCMS. ¹H-NMR (400 MHz, DMSO-d₆) δ ppm: 8.83 (1H, s), 8.60 (1H, d, J=2 Hz), 8.12-8.06 (1H, m), 7.89 (1H, s), 6.79 (1H, s), 2.66 (3H, s), 2.09 (3H, s), 1.91 (3H, s). MS(ES) m/z 422.17 (M+H).

Step 8: Preparation of (P)-(E)-3-chloro-4-((3,5-difluoropyridin-2-yl)methoxy-d2)-2′-(3-(dimethylamino)acryloyl)-5′,6-dimethyl-2H-[1,4′-bipyridin]-2-one (CPD-13)

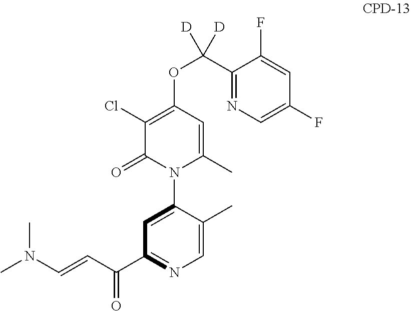

To a stirred suspension of (P)-2′-acetyl-3-chloro-4-((3,5-difluoropyridin-2-yl)methoxy-d2)-5′,6-dimethyl-2H-[1,4′-bipyridin]-2-one (CPD-12) (Example 7, Step 7, 6.5 g, 15.4 mmol, 1.0 eq., (Isomer I:Isomer II) 98.79%: 1.21% (97.58% ee) in DMF (6.5 ml, 1 vol) was added DMF-DMA (5.5 g, 7.12 mmol, 3 eq.) at 25-30° C. Then reaction mass was heated to 55-60° C. and maintained for 18 h (Note—throughout the reaction mass is seen as a suspension). Reaction progress was monitored by TLC/HPLC and upon completion the reaction mixture was cooled to RT and diluted with ethyl acetate (13 ml, 2.0 vol) stirred for 1 h. Solid was filtered and washed with EtOAc (6.5 ml, 1 vol). It was suck dried well and then dried under vacuum at 45° C. for 1 h to afford (P)-(E)-3-chloro-4-((3,5-difluoropyridin-2-yl)methoxy-d2)-2′-(3-(dimethylamino)acryloyl)-5′,6-dimethyl-2H-[1,4′-bipyridin]-2-one (CPD-13) as a pale yellow solid 5.8 g (yield=79%) with 99.15% purity by HPLC, (Isomer I:Isomer II) 99.34%: 0.66% (98.68% ee) chiral purity by chiral HPLC, and 96.01% D2-purity by LCMS. ¹H-NMR (400 MHz), DMSO-d₆ δ ppm: 8.71 (1H, s), 8.60 (1H, d, J=2 Hz), 8.11-8.06 (1H, m), 7.85 (1H, s), 7.85 (1H, d, J=12.8 Hz), 7.81 (1H, s), 6.78 (1H, s), 6.38 (1H, d, J=12.8 Hz), 3.19 (3H, s), 2.94 (3H, s), 2.05 (3H, s), 1.91 (3H, s). MS(ES) m/z 477.25 (M+H).

Step 9: (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I)

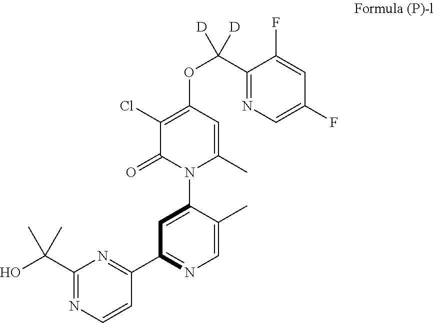

To a stirred solution of (P)-(E)-3-chloro-4-((3,5-difluoropyridin-2-yl)methoxy-d2)-2′-(3-(dimethylamino)acryloyl)-5′,6-dimethyl-2H-[1,4′-bipyridin]-2-one (CPD-13) (Example 7, Step 8, 5 g, 10.4 mmol, 1 eq., (Isomer I:Isomer II) 99.34%:0.66% (98.68% ee) in DMF (30 ml, 6 vol) potassium carbonate (3.6 g, 26.2 mmol, 2.5 eq,) was added portion wise at RT. Reaction mass was stirred for 30 min, 1-amino-2-hydroxy-2-methylpropan-1-iminium (INT-02) (4.3 g, 31.4 mmol, 3.0 eq.) was added portion wise at RT. The reaction mass was heated at 45-50° C. (inner temp) for 18 h. Homogenous solution was observed after 18 h. Reaction progress was monitored by HPLC & TLC and upon completion the reaction mixture was cooled to 10-15° C. and stirred for 30 min. Ice-cold Water (100 ml, 20 vol) was added to get solid. Further stirred for 1 h at 10-15° C. Solid was filtered, washed with water (50 ml, 10 vol) and dried under vacuum to afford crude (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I) with 90% purity by HPLC. The crude material was co-distilled with MeOH (2×25 ml, 5 vol) and dried. The solid (5.5 g), was taken in rbf, methanol (33 ml, 6 vol) was added and heated at 55-60° C. to afford clear solution. It was cooled to 10-15° C. and stirred for 1 h. It was filtered and washed with methanol (5 ml), dried under vacuum to get (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I) as an off white solid 4.0 g (yield=74.2%) with 98.92% purity by HPLC, (Isomer I:Isomer II) 99.47%:0.53% (98.94% ee) purity by chiral HPLC, and 95.84% D2-purity by LCMS. ¹H-NMR (400 MHz), DMSO-d₆ δ ppm: 8.97 (1H, d, J=5.2 Hz), 8.86 (1H, s), 8.69 (1H, s), 8.61 (1H, d, J=2.4 Hz), 8.24 (1H, d, J=5.2 Hz), 8.12-8.07 (1H, m), 6.84 (1H, s), 5.24 (1H, s), 2.10 (3H, s), 1.98 (3H, s), 1.53 (6H, s). MS(ES) m/z 516.29 (M+H).

### Example 8: Preparation of racemic 2-(chloromethyl-d)-3,5-difluoropyridine (INT-10)

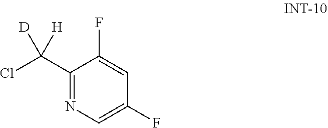 [SMILES: [2H]C(Cl)c1ncc(F)cc1F]

Step 1: Synthesis of (3,5-difluoropyridin-2-yl)methanol (INT-E)

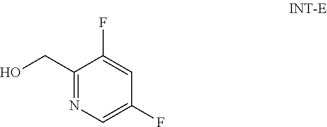

methyl 3,5-difluoropicolinate (INT-D) (104 g, 0.600 mol, 1 eq) was dissolved in methanol (104 ml, 1 vol) and THF (208 ml, 2 vol) at 25-30° C. The reaction mass was cooled to 0-5° C. using ice-bath. Sodium borohydride (34 g, 0.9011 mol, 1.5 eq) was added portion wise below 0-10° C. Reaction was exothermic and controlled by slow addition of sodium borohydride, maintain the temperature at 10-15° C. Reaction mass was stirred at 10-15° C. for 1 h. Reaction progress was monitored by TLC. After reaction completion the reaction was quenched with ice cold water (520 ml, 5 vol). It was extracted with ethyl acetate (416 ml×4). (Note: Emulsion formed, then filtered through celite bed). The organic layer was dried over anhydrous sodium sulfate and concentrated in vacuo to afford (3,5-difluoropyridin-2-yl)methanol (INT-E) as a colorless liquid 84 g (yield=96.3%) with 97.34% purity by HPLC. ¹H-NMR (400 MHz, CDCl₃) δ ppm: 8.33 (1H, d, J=2.4 Hz), 7.25-7.20 (1H, m), 4.81 (2H, s), 3.57 (1H, bs). MS(ES) m/z 145.84 (M+H).

Step 2: Synthesis of 3,5-difluoropicolinaldehyde (INT-F)

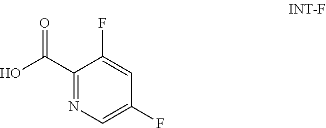 [SMILES: O=C(O)c1ncc(F)cc1F]

3,5-difluoropyridin-2-yl)methanol (INT-E) (Example 8, Step 1, 77 g, 0.531 mol, 1 eq) in dichloromethane (1.540 L, 20 vol) was added Dess-Martin Periodinane (382.7 g, 0.902 mol, 1.7 eq) at 0-5° C. portion wise. Reaction mass was stirred for 2 h at rt. Reaction progress was monitored by TLC. After completion the reaction cooled to 5-10° C. Quenched with saturated NaHCO3 solution (15 vol), stirred for 30 minutes. Filtered the solids and washed with DCM (770 ml, 10 vol). Separated the two layers, aqueous layer was extracted with DCM and the combined organic layer was dried over Na₂SO₄ and evaporated to afford crude 3,5-difluoropicolinaldehyde (INT-F). The crude compound was purified by FCC over Si-gel, the pure fractions were pooled and evaporated to afford 3,5-difluoropicolinaldehyde (INT-F) as a pale yellow solid 53 g (yield=63%) with 97.08 purity by HPLC. ¹H-NMR (400 MHz, CDCl₃) δ ppm: 10.15 (1H, s), 8.53 (1H, d, J=2.4 Hz), 7.39-7.34 (1H, m).

Step 3: Synthesis of racemic (3,5-difluoropyridin-2-yl)methan-d-ol (INT-G)

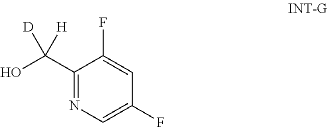 [SMILES: [2H]C(O)c1ncc(F)cc1F]

3,5-difluoropicolinaldehyde (INT-F) (Example 8, Step 2, 31 g, 0.216 mmol, 1.0 eq)) in THF (62 ml, 2 vol) and MeOD (31 ml, 1 vol) was added NaBD₄ (4.5 g, 0.108 mol, 0.5 eq) at 0° C. Reaction mass was stirred at 10-15° c. for 30 minutes. Reaction progress was monitored by TLC until complete then reaction mass was quenched with ice-cold water (150 ml), diluted with ethyl acetate (150 ml) and stirred for 10 minutes. Separated the two layers and aqueous layer was extracted with ethyl acetate (150 ml). The combined organic layer was dried over Na₂SO₄ and evaporated to afford racemic (3,5-difluoropyridin-2-yl)methan-d-ol (INT-G) as a pale yellow liquid 52 g (yield=90%) with 99.83% purity by HPLC, 98.12% D1-purity by LCMS. ¹H-NMR (400 MHz, CDCl₃) δ ppm: 8.46 (1H, d, J=2.4 Hz), 7.87-7.93 (1H, m), 5.33 (1H, d, J=2.0 Hz), 4.54-4.55 (1H, m). MS(ES) m/z 146.91 (M+H).

Step 4: Synthesis of racemic 2-(chloromethyl-d)-3,5-difluoropyridine (INT-10)

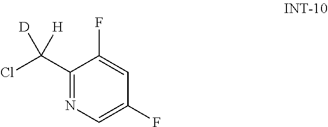 [SMILES: [2H]C(Cl)c1ncc(F)cc1F]

To a stirred solution of racemic (3,5-difluoropyridin-2-yl)methan-d-ol (INT-G) (Example 8, Step 3, 52 g, 0.356 mol) in dichloromethane (260 ml, 5 vol), was added thionyl chloride (39 ml, 0.534 mol, 1.5 eq) and N,N-dimethylformamide (2.6 ml, 0.05 vol) at 0° C. Ice-bath was removed and the resulting solution was stirred at rt for 3 hours. The pH of reaction mixture was adjusted to 7-8 with saturated sodium bicarbonate solution (1.3 L) at 0-10° C. It was extracted with dichloromethane (1 L×2). Combined extracts were wash with saturated sodium bicarbonate solution (260 mL, 5 vol) and wash with water (500 ml). Organic layer dried over anhydrous sodium sulfate (60 g) and concentrated in vacuo to afford racemic 2-(chloromethyl-d)-3,5-difluoropyridine (INT-10) as a pale yellow liquid 54 g (yield=94%) with 99.33% purity by HPLC, 96.65 D1-purity by LCMS. ¹H-NMR (400 MHz, CDCl₃) δ ppm: 8.53 (1H, s), 8.01-8.07 (1H, m), 4.82 (1H, d, J=2.0 Hz). MS(ES) m/z 165.01 (M+H).

### Example 9: Preparation of (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II)

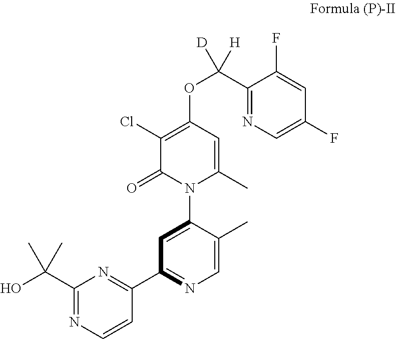

Step 1: Preparation of 2′-chloro-4-((3,5-difluoropyridin-2-yl)methoxy-d)-5′,6-dimethyl-2H-[1,4′-bipyridin]-2-one (CPD-16)

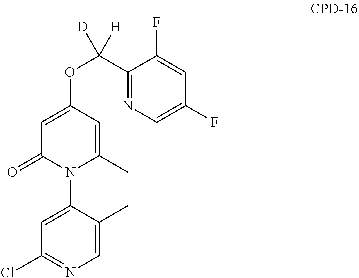 [SMILES: [2H]C(Oc1cc(C)n(-c2cc(Cl)ncc2C)c(=O)c1)c1ncc(F)cc1F]

A suspension of 2′-chloro-4-hydroxy-5′,6-dimethyl-2H-[1,4′-bipyridin]-2-one (CPD-02) (50 g, 0.2 mol, 1.0 eq) and potassium carbonate powder (50 g, 0.36 mol 1.8 eq) in N,N-dimethylformamide (250 ml) was mechanically stirred for 30 min at rt. A solution of racemic 2-(chloromethyl-d)-3,5-difluoropyridine (INT-10) (39.36 g, 0.24 mol, 1.2 eq) in N,N-dimethylformamide (50 ml) was added at ambient temperature for about 30 minutes. The reaction mixture was stirred at rt for 48 hours. The progress of the reaction was monitored by HPLC/TLC. After completion the reaction mas was filtered and filtrate was poured into ice cold water (1000 ml). Reaction mass was diluted with ethyl acetate (500 ml), stirred for 30 minutes. Separated the two layers and aqueous layer was again extracted with Ethyl acetate (250 ml). Combined organic layer and washed with water (2×500 ml) and brine solution (250 ml), and dried over sodium sulphate. Solvent was distilled up to −50 ml under vacuum at 45° C. MTBE (500 ml) was added and reaction mass was co-distilled up to −100 ml. MTBE was added (500 ml) and stirred for 4 h at rt. The reaction mass was filtered and washed with MTBE (100 ml). The filter cake was dried well to afford crude 2′-chloro-4-((3,5-difluoropyridin-2-yl)methoxy-d)-5′,6-dimethyl-2H-[1,4′-bipyridin]-2-one (CPD-16) as an off-white solid. The crude was purified by Prep HPLC in Ammonium bicarbonate and acetonitrile and then lyophilized to afford 2′-chloro-4-((3,5-difluoropyridin-2-yl)methoxy-d)-5′,6-dimethyl-2H-[1,4′-bipyridin]-2-one (CPD-16) as an off-white solid 49 g (yield=64.85%) with 99.58% purity by HPLC, 98.39 D1-purity by LCMS as off-white solid. ¹H-NMR (400 MHz, DMSO-d₆) δ ppm: 8.59 (1H, d, J=2 Hz), 8.49 (1H, s), 8.09-8.05 (1H, m), 7.60 (1H, s), 6.13 (1H, d, J=1.6 Hz), 6.02 (1H, d, J=2.8 Hz), 5.22 (1H, bs), 1.98 (3H, s), 1.84 (3H, s). MS(ES) m/z 378.96 (M+H).

Step 2: Preparation of 2′-acetyl-4-((3,5-difluoropyridin-2-yl)methoxy-d)-5′,6-dimethyl-2H-[1,4′-bipyridin]-2-one (CPD-17)

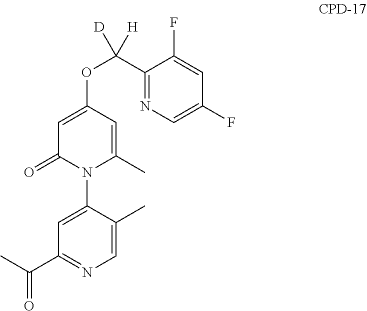 [SMILES: [2H]C(Oc1cc(C)n(-c2cc(C(C)=O)ncc2C)c(=O)c1)c1ncc(F)cc1F]

2′-chloro-4-((3,5-difluoropyridin-2-yl)methoxy-d)-5′,6-dimethyl-2H-[1,4′-bipyridin]-2-one (CPD-16) (Example 9, Step 1, 30 g, 79.36 mmol, 1 eq) was taken into 2 L three-neck RBF and suspended in 1,4-dioxane (225 ml, 7.5 vol). Tributyl (1-ethoxy vinyl) tin (42.98 g, 119.04 mmol, 1.5 eq) was added at rt under argon atmosphere. Reaction mass was degassed with argon for 20 minutes. Then CuI (0.755 g, 3.96 mmol, 0.05 eq) was added. Reaction mass was degassed with argon for 20 minutes. Then Pd(dppf)Cl₂.DCM complex (3.23 g, 3.96 mol, 0.05 eq) was added under argon atmosphere. Reaction mass was degassed with argon for 10 minutes. Then reaction was heated to 100-105° C. for 14 h and the reaction progress was monitored by HPLC which showed 94.5% desired ethoxyvinyl ether product. 2 g of the reaction mixture was taken and purified by medium pressure MPLC in neutral conditions using water in acetonitrile. The combined pure fractions were evaporated and extracted with ethyl acetate. The organic layer was dried over Na₂SO₄ and evaporated to afford 4-((3,5-difluoropyridin-2-yl)methoxy-d)-2′-(1-ethoxyvinyl)-5′,6-dimethyl-2H-[1,4′-bipyridin]-2-one (CPD-29) as a light brown solid 510 mg (yield=%) with 98.76% purity by HPLC, 97.97% D1-purity by LCMS. 1-H-NAIR (400 MHz, DMSO-d₆) δ ppm: 8.60 (2H, m), 8.09-8.04 (1H, m), 7.39 (1H, s), 6.11 (1H, s), 6.02 (1H, d, J=2.4 Hz), 5.40 (1H, s), 5.22 (1H, bs), 4.44 (1H, s), 3.96 (2H, q, J=7.2 Hz), 2.0 (3H, s), 1.81 (3H, s), 1.36 (3H, t, J=6.8 Hz). MS(ES) m/z 415.21 (M+H). The remaining reaction mixture was cooled to rt, and cooled to 0-5° C. Conc HCl (30 ml, 1 vol) was added below 10° C. After completion of addition removed the cooling bath and stirred at rt for 2 h. Reaction progress was monitored by TLC and HPLC. Reaction mass was basified with 20% of aqueous sodium hydroxide solution to pH 9-10. Extracted with ethyl acetate (2×250 ml). The crude compound was purified by FCC over si-gel. The pure fractions were evaporated to afford crude 2′-acetyl-4-((3,5-difluoropyridin-2-yl)methoxy-d)-5′,6-dimethyl-2H-[1,4′-bipyridin]-2-one (CPD-17) as a pale brown solid. The crude material was redissolved in EtOAc (200 ml), washed with 2M KF solution (2×150 ml) and water (2×150 ml). It was dried over Na₂SO₄ and washed with EtOAc (60 ml, 2 vol), distilled under vacuum at 45-50° C. up to 50 ml. Co-distilled sequentially with 30% of EtoAc/pet ether (4×150 ml) and 20% of EtOAc/pet ether (2×100 ml). 20% of EtOAc/pet ether (60 ml, 2 vol) was charged and stirred for 2-3 h at rt. Filtered and rinsed with 20% of EtOAc/pet ether (60 ml, 2 vol) under vacuum to afford 2′-acetyl-4-((3,5-difluoropyridin-2-yl)methoxy-d)-5′,6-dimethyl-2H-[1,4′-bipyridin]-2-one (CPD-17) as an off-white solid 21 g (yield=68.3%) with 99.82 purity by HPLC, 97.90% D1-purity by LCMS. III-NMR (400 MHz, DMSO-d₆) δ ppm: 8.79 (1H, s), 8.59 (1H, d, J=2.4 Hz), 8.10-8.04 (1H, m), 7.78 (1H, s), 6.13 (1H, d, J=1.6 Hz), 6.03 (1H, d, J=2.4 Hz), 5.22 (1H, s), 2.65 (3H, s), 2.09 (3H, s), 1.80 (3H, s). MS(ES) m/z 387.15 (M+H).

Step 3: Preparation of 2′-acetyl-3-chloro-4-((3,5-difluoropyridin-2-yl)methoxy-d)-5′,6-dimethyl-2H-[1,4′-bipyridin]-2-one (CPD-18)

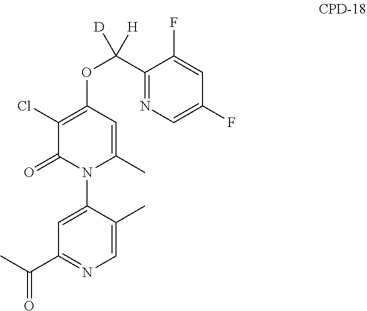 [SMILES: [2H]C(Oc1cc(C)n(-c2cc(C(C)=O)ncc2C)c(=O)c1Cl)c1ncc(F)cc1F]

To a stirred suspension of 2′-acetyl-4-((3,5-difluoropyridin-2-yl)methoxy-d)-5′,6-dimethyl-2H-[1,4′-bipyridin]-2-one (CPD-17) (Example 9, Step 2, 18 g, 0.046 mol, 1.0 eq) in IPA (270 ml, 15 vol) was added N-chlorosuccinimide (7.4 g, 0.055 mol, 1.2 eq) portion wise at rt. Then reaction was heated to 65-70° C. and maintained for 2 h. When temperature was reached to 55-60° C. clear solution was formed and after 1 h solid formation was observed. Reaction progress was monitored by TLC. After reaction completion the reaction mixture was cooled to rt and stirred for 30 min. Solid was filtered, washed with IPA (36 ml, 2 vol) and dried under vacuum at 45° C. for 1 h to afford crude 2′-acetyl-3-chloro-4-((3,5-difluoropyridin-2-yl)methoxy-d)-5′,6-dimethyl-2H-[1,4′-bipyridin]-2-one (CPD-18) as an off-white solid 14.7 g (yield=75%). 1 g of solid compound was suspended in Methanol (10 ml) and heated to 60° C. for 1 h and cooled and filtered to afford 2′-acetyl-3-chloro-4-((3,5-difluoropyridin-2-yl)methoxy-d)-5′,6-dimethyl-2H-[1,4′-bipyridin]-2-one (CPD-18) as an off-white solid 560 mg (yield=51.85%) with 98.78% purity by HPLC, 97.97% D1-purity by LCMS. ¹H-NMR (400 MHz, DMSO-d₆) δ ppm: 8.83 (1H, s), 8.60 (1H, d, J=2.4 Hz), 8.12-8.06 (1H, m), 7.89 (1H, s), 6.79 (1H, s), 5.45 (1H, s), 2.66 (3H, s), 2.09 (3H, s), 1.91 (3H, s). MS(ES) m/z 421.21 (M+H).

Step 4: Preparation of (E)-3-chloro-4-((3,5-difluoropyridin-2-yl)methoxy-d)-2′-(3-(dimethylamino)acryloyl)-5′,6-dimethyl-2H-[1,4′-bipyridin]-2-one (CPD-19)

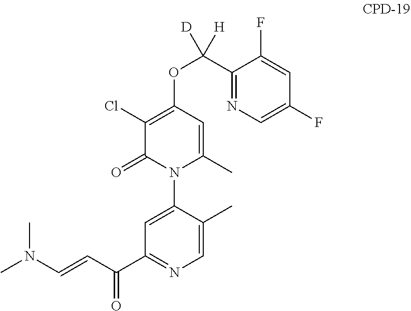 [SMILES: [2H]C(Oc1cc(C)n(-c2cc(C(=O)/C=C/N(C)C)ncc2C)c(=O)c1Cl)c1ncc(F)cc1F]

To a stirred suspension of 2′-acetyl-3-chloro-4-((3,5-difluoropyridin-2-yl)methoxy-d)-5′,6-dimethyl-2H-[1,4′-bipyridin]-2-one (CPD-18) (Example 9, Step 3, 12 g, 0.028 mol, 1.0 eq) in DMF (12 ml, 1 vol) was added DMF-DMA (10.2 g, 0.085 mol, 3 eq) at 25-30° C. Then reaction mass was heated to 55-60° C. and maintained for 16 h (Note—throughout the reaction, reaction mass was a suspension). Reaction progress was monitored by TLC/HPLC. After reaction completion the reaction mixture was cooled to RT and diluted with ethyl acetate (18 ml, 1.5 vol) and stirred for 30-45 min. Solid was filtered and washed with EtOAc (12 ml). It was suck dried well and then dried under vacuum at 45° C. for 1 h to afford (E)-3-chloro-4-((3,5-difluoropyridin-2-yl)methoxy-d)-2′-(3-(dimethylamino)acryloyl)-5′,6-dimethyl-2H-[1,4′-bipyridin]-2-one (CPD-19) as a pale yellow solid 11 g (yield=81%) with 98.18% purity by HPLC, 97.88% D1-purity by LCMS. ¹H-NMR (400 MHz, DMSO-d₆) δ ppm: 8.71 (1H, s), 8.60 (1H, d, J=2.4 Hz), 8.11-8.06 (1H, m), 7.85 (1H, s), 7.82 (1H, d, J=3.2 Hz), 6.78 (1H, s), 6.38 (1H, d, J=12.8 Hz), 3.19 (3H, s), 2.94 (3H, s), 2.05 (3H, s), 1.91 (3H, s). MS(ES) m/z 476.24 (M+H).

Step 5: Preparation of 3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (CPD-20)

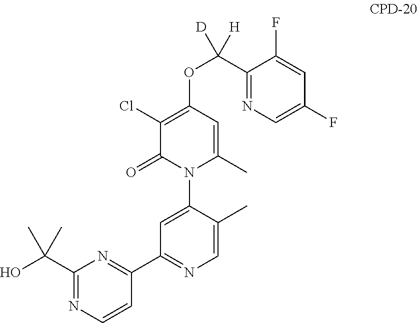 [SMILES: [2H]C(Oc1cc(C)n(-c2cc(-c3ccnc(C(C)(C)O)n3)ncc2C)c(=O)c1Cl)c1ncc(F)cc1F]

To a stirred solution of (E)-3-chloro-4-((3,5-difluoropyridin-2-yl)methoxy-d)-2′-(3-(dimethylamino)acryloyl)-5′,6-dimethyl-2H-[1,4′-bipyridin]-2-one (CPD-19) (Example 9, Step 4, 8 g, 0.8389 mol, 1.0 eq) in DMF (48 ml, 6 vol), potassium carbonate (5.9 g, 0.043 mol, 2.5 eq) was added portion wise at RT. Reaction mass was stirred for 30 min, 1-amino-2-hydroxy-2-methylpropan-1-iminium (INT-02) (8 g, 0.050 mol, 3.0 eq) was added portion wise at RT. The reaction mass was heated at 45-50° C. (inner temp) for 12-14 h. Homogenous solution was observed after 12 h. Reaction progress was monitored by HPLC & TLC. After reaction completion the reaction mixture was cooled to 10-15° C. and stirred for 30 min. Ice-cold water (20 vol) was added to get solid. Further stirred for 1 h at 10-15° C. Solid was filtered, washed with water (10 vol) and dried under vacuum. Then co-distilled with MeOH to get solid (11 g). It was taken in rbf added (110 mL, 10 vol) of MeOH and heated at 65-70° C. to afford clear solution. It was cooled to 10-15° C. and stirred for 1 h. It was filtered and washed with methanol (10 ml), dried under vacuum to afford 3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (CPD-20) (as an off-white solid 5.4 g (yield=62.7%) with 98.09% purity by HPLC, 97.67% D1-purity by LCMS. 1H-NMR (400 MHz, DMSO-d₆) δ ppm: 8.97 (1H, d, J=5.2 Hz), 8.86 (1H, s), 8.68 (1H, s), 8.61 (1H, d, J=2.4 Hz), 8.24 (1H, d, J=5.2 Hz), 8.12-8.07 (1H, m), 6.84 (1H, s), 5.47 (1H, s), 5.24 (1H, S), 2.10 (3H, s), 1.98 (3H, s), 1.53 (3H, s), 1.52 (3H, s). MS(ES) m/z 512.29 (M+H).

Step 6: Preparation of (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II) and (M)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (M)-II)

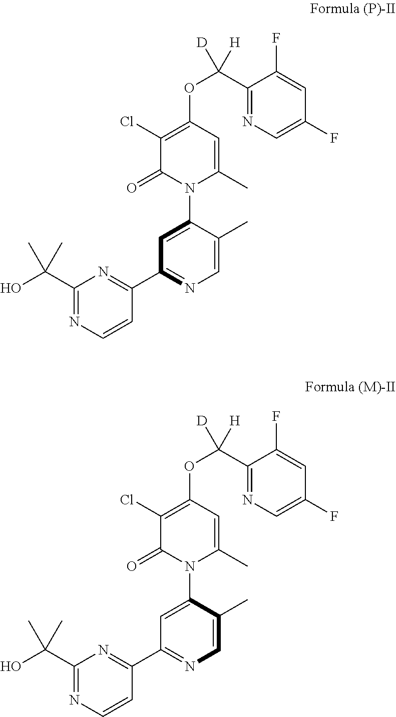

3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (CPD-20) (Example 9, Step 5, 4.5 g) was purified by SFC purification. Collected peak-1 and peak-2 separate fractions and evaporated to afford (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II) (Peak-1)=2.0 g, Formula (M)-II (Peak-2)=1.9 g.

Step 7: Preparation of (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II)

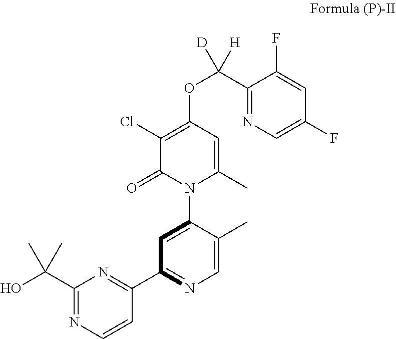

A suspension of (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II) (Example 9, Step 6, Peak-1, 2 g, 3.88 mmol, 1 eq.) in isopropyl alcohol (50 ml, 25 vol) was heated to 65-70° C. and stirred for 1.0 h (a clear solution was observed). Reaction mass was filtered hot at 65-70° C. Filtrate allowed to RT and stirred for 16 h. The reaction mass was filtered the solid and washed with isopropyl alcohol (5 ml, 2.5 vol). The solid was dried under vacuum at 40° C. to afford (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II) as off white solid 1.55 g (yield=77.5%) with 99.81% purity by HPLC, (Isomer I:Isomer II) 99.99%: 0.01% (99.98 ee), 97.79% D1-purity by LCMS. ¹H-NMR (400 MHz, DMSO-d₆) δ ppm: 8.97 (1H, d, J=5.2 Hz), 8.86 (1H, s), 8.68 (1H, s), 8.61 (1H, d, J=2.4 Hz), 8.24 (1H, d, J=5.2 Hz), 8.12-8.07 (1H, m), 6.84 (1H, s), 5.47 (1H, s), 5.24 (1H, S), 2.10 (3H, s), 1.98 (3H, s), 1.53 (3H, s), 1.52 (3H, s). MS(ES) m/z 512.25 (M+H).

### Example 10: Preparation of (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II) via chiral separation with (S)-2-naphthylethyl amine

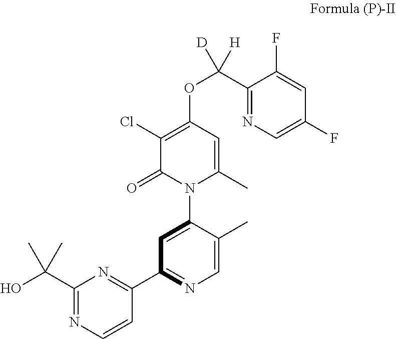

Step 1: Preparation of 2′,3-dichloro-4-((3,5-difluoropyridin-2-yl)methoxy-d)-5′,6-dimethyl-2H-[1,4′-bipyridin]-2-one (CPD-21)

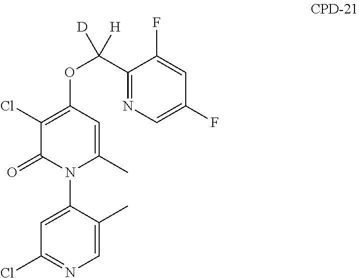 [SMILES: [2H]C(Oc1cc(C)n(-c2cc(Cl)ncc2C)c(=O)c1Cl)c1ncc(F)cc1F]

To a stirred solution of 2′-chloro-4-((3,5-difluoropyridin-2-yl)methoxy-d)-5′,6-dimethyl-2H-[1,4′-bipyridin]-2-one (CPD-16) (Example 9, Step 1, 55 g, 14.52 mol, 1.0 eq) in IPA (825 ml, 15 vol), N-chlorosuccinimide (23.26 g, 17.42 mol, 1.2 eq) was added portion wise at RT. The reaction mixture was heated to 65-70° C. and maintained for 2 h. Clear solution was formed and after 1 h, and then solid formation was observed. Reaction progress was monitored by TLC. After reaction completion the reaction mixture was cooled to RT and stirred for ˜45 min. It was filtered, washed with IPA (55 ml) and dried under vacuum at 45° C. for 3 h to afford 2′,3-dichloro-4-((3,5-difluoropyridin-2-yl)methoxy-d)-5′,6-dimethyl-2H-[1,4′-bipyridin]-2-one (CPD-21) (46.2 g, 77%) as off white solid with 98.82% purity by HPLC, 97.41% D1-purity by LCMS. ¹H-NMR (400 MHz, DMSO-d₆) δ ppm: 8.60 (1H, d, J=2.4 Hz), 8.53 (1H, s), 8.11-8.06 (1H, m), 7.68 (1H, s), 6.79 (1H, s), 5.54 (1H, s), 1.98 (3H, s), 1.95 (3H, s). MS(ES) m/z 413.11 (M+H).

Step 2: Preparation of 3-chloro-4-((3,5-difluoropyridin-2-yl)methoxy-d)-5′,6-dimethyl-2-oxo-2H-[1,4′-bipyridine]-2′-carboxylic acid (CPD-22).

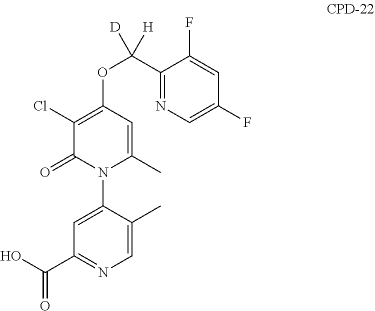 [SMILES: [2H]C(Oc1cc(C)n(-c2cc(C(=O)O)ncc2C)c(=O)c1Cl)c1ncc(F)cc1F]

A clean and dried 2 L autoclave flask was de-poisoned as follows: A mixture of 2′,3-dichloro-4-((3,5-difluoropyridin-2-yl)methoxy-d)-5′,6-dimethyl-2H-[1,4′-bipyridin]-2-one (CPD-21) (Example 10, Step 1, 500 mg), Pd(dppf)Cl₂ (500 mg), acetonitrile (250 ml) and water (250 ml.) was stirred under CO pressure 40-50 psi for about 1 h at 75° C. The vessel was emptied and rinsed with acetonitrile. The rinse was discarded. 2′,3-dichloro-4-((3,5-difluoropyridin-2-yl)methoxy-d)-5′,6-dimethyl-2H-[1,4′-bipyridin]-2-one (CPD-21) (Example 10, Step 1, 45 g, 10.89 mol, 1.0 eq), acetonitrile (360 ml, 8.0 vol.) and water (180 ml, 4.0 vol.) were charged in the autoclave at rt. Li₂CO₃ (24.14 g, 32.68 mol, 3.0 eq.) was added and the mixture was purged with argon for 30 min and followed by addition of Pd(dppf)Cl₂ (4.41 g, 0.054 mol, 0.05 eq). The reaction mass was further purged with argon for 15 minutes. The reaction vessel was closed and pressurised with CO (40-45 psi) and stirred for a minute. Pressure was released and again applied CO pressure 100 psi (5.0 kg). The reaction mixture was mechanically stirred and heated at 75° C. for 36 h. The progress of the reaction was monitored by TLC/HPLC. After reaction completion the reaction mass was cooled to 25-30° C. and pressure was released. Reaction mixture was de-gassed with argon and unloaded. Water (225 ml, 5 vol) was added and pH was adjusted to −14 with 2N NaOH solution. Then reaction mass was washed with MTBE (3×500 ml). Aqueous layer was filtered through hyflow bed. The pH of filtered mL's was adjusted to −1-2 with 6 N HCl and stirred for ˜2 h at 25-30° C. The precipitated solid was filtered. It was washed with water (450 ml, 10 vol.) followed by ethanol (45 ml, 1 vol.) and dried under vacuum to afford 3-chloro-4-((3,5-difluoropyridin-2-yl)methoxy-d)-5′,6-dimethyl-2-oxo-2H-[1,4′-bipyridine]-2′-carboxylic acid (CPD-22) as an off-white solid 40 g (yield=86.8%) with 99.46% purity by HPLC, 97.27% D1-purity by LCMS. ¹H-NMR (400 MHz, DMSO-d₆) δ ppm: 13.36 (1H, s), 8.80 (1H, s), 8.60 (1H, d, J=2.4 Hz), 8.12-8.06 (1H, m), 7.96 (1H, s), 6.79 (1H, s), 5.45 (1H, s), 2.07 (3H, s), 1.92 (3H, s). MS(ES) m/z 423.14 (M+H).

Step 3: Preparation of (P)-3-chloro-4-((3,5-difluoropyridin-2-yl)methoxy-d)-5′,6-dimethyl-2-oxo-2H-[1,4′-bipyridine]-2′-carboxylate (S)-1-(naphthalen-1-yl)ethan-1-aminium (Isomer I-salt) (CPD-23 Salt A) and (M)-3-chloro-4-((3,5-difluoropyridin-2-yl)methoxy-d)-5′,6-dimethyl-2-oxo-2H-[1,4′-bipyridine]-2′-carboxylate (S)-1-(naphthalen-1-yl)ethan-1-aminium (Isomer II-salt) (CPD-30 Salt A)

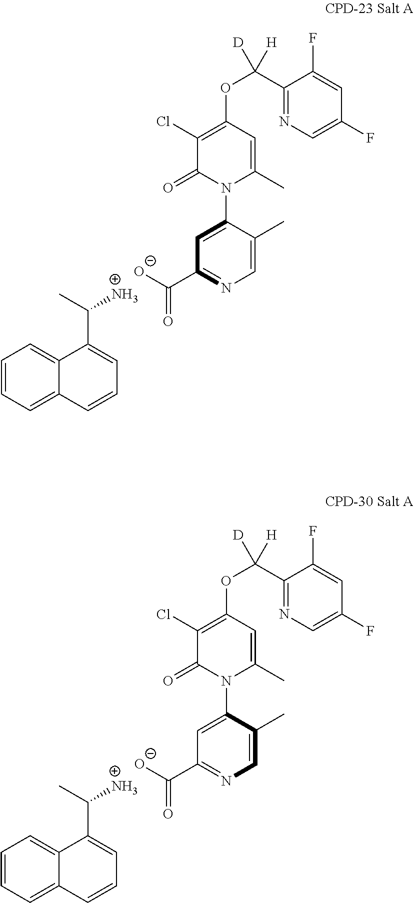

A stirred suspension of 3-chloro-4-((3,5-difluoropyridin-2-yl)methoxy-d)-5′,6-dimethyl-2-oxo-2H-[1,4′-bipyridine]-2′-carboxylic acid (CPD-22) (Example 10, Step 2, 20 g, 4.739 mmol, 1.0 eq) in 5% of DMSO: anisole (140 ml, 7 vol) and slowly heated to 110-115° C. The reaction mass was stirred for 10-15 min. A solution of (S)-2-naphthylethyl amine (8.44 g, 49.289 mmol, 1.04 eq.) in 5% of DMSO: anisole (280 ml, 14 vol.) was slowly added to the reaction mass over 30 min. The reaction was stirred for 72 h at 110-115° C. Reaction was monitored by Chiral HPLC; chiral HPLC@72 h—(Isomer I:Isomer II) 98.84%: 1.16% (97.68% ee). The above reaction mixture was allowed to rt, filtered the solid, washed with petroleum ether (200 ml×3), and dried under vacuum for 4 h at 40° C. to get (P)-3-chloro-4-((3,5-difluoropyridin-2-yl)methoxy-d)-5′,6-dimethyl-2-oxo-2H-[1,4′-bipyridine]-2′-carboxylate (S)-1-(naphthalen-1-yl)ethan-1-aminium (Isomer I-salt) (CPD-23 Salt A) and (M)-3-chloro-4-((3,5-difluoropyridin-2-yl)methoxy-d)-5′,6-dimethyl-2-oxo-2H-[1,4′-bipyridine]-2′-carboxylate (S)-1-(naphthalen-1-yl)ethan-1-aminium (Isomer II-salt) (CPD-30 Salt A) as a white solid 22 g (yield=78.5%) with (Isomer I:Isomer II) 97.64%:2.36% (95.28% ee).

Step 4: Preparation of (P)-3-chloro-4-((3,5-difluoropyridin-2-yl)methoxy-d)-5′,6-dimethyl-2-oxo-2H-[1,4′-bipyridine]-2′-carboxylate (Isomer I) (CPD-23) and (M)-3-chloro-4-((3,5-difluoropyridin-2-yl)methoxy-d)-5′,6-dimethyl-2-oxo-2H-[1,4′-bipyridine]-2′-carboxylate (Isomer II) (CPD-30)

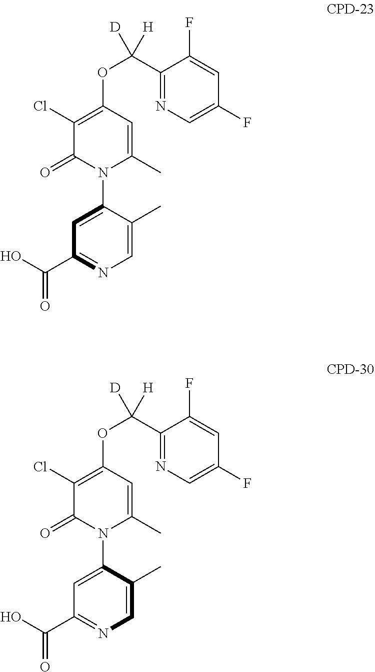

(P)-3-chloro-4-((3,5-difluoropyridin-2-yl)methoxy-d)-5′,6-dim ethyl-2-oxo-2H-[1,4′-bipyridine]-2′-carboxylate (S)-1-(naphthalen-1-yl)ethan-1-aminium (Isomer I-salt) (CPD-23 Salt A) and (M)-3-chloro-4-((3,5-difluoropyridin-2-yl)methoxy-d)-5′,6-dimethyl-2-oxo-2H-[1,4′-bipyridine]-2′-carboxylate (S)-1-(naphthalen-1-yl)ethan-1-aminium (CPD-30 Salt A) (Example 10, Step 3, 21 g, 35.36 mmol, 1.0 eq, (Isomer I:Isomer II) 97.64%:2.36% (95.28 ee)) were dissolved in water (630 ml, 30 vol.) and basified with 2N NaOH (63 ml, 3 vol) to pH˜14 and extracted with MTBE (3×210 ml). Aqueous layer was filtered through celite, washed with MTBE (105 ml, 5 vol). It was acidified with 6N HCl (52.5 ml, 2.5 vol) to pH ˜2 and stirred for 2 h. Precipitated solid was filtered, washed with water (10 vol) and dried under vacuum to afford (P)-3-chloro-4-((3,5-difluoropyridin-2-yl)methoxy-d)-5′,6-dimethyl-2-oxo-2H-[1,4′-bipyridine]-2′-carboxylate (Isomer I) (CPD-23) and (M)-3-chloro-4-((3,5-difluoropyridin-2-yl)methoxy-d)-5′,6-dim ethyl-2-oxo-2H-[1,4′-bipyridine]-2′-carboxylate (Isomer II) (CPD-30) as a white solid 13 g (yield=87.2%) with (Isomer I:Isomer II) 97.37:2.63% (94.74 ee) chiral purity by chiral HPLC. Proceeded for enrichment of once again. A stirred suspension of (P)-3-chloro-4-((3,5-difluoropyridin-2-yl)methoxy-d)-5′,6-dimethyl-2-oxo-2H-[1,4′-bipyridine]-2′-carboxylate (Isomer I) (CPD-23) obtained above (13 g, 30.74 mmol, 1.0 eq) in 5% of DMSO: anisole (91 ml, 7 vol) was stirred for 10-15 min. A solution of (S)-2-naphthylethyl amine (5.47 g, 31.97 mmol, 1.04 eq.) in 5% of DMSO: anisole (182 ml, 14 vol.) was slowly added to the reaction mass at RT, over 30 min. The reaction was then heated to 110-115° C., stirred for 4 h at 110-115° C., Reaction was monitored by Chiral HPLC. After completion the reaction mixture was allowed to rt, filtered the solid, washed with pet-ether (3×130 mL), dried under vacuum for 4 h at 40° C. to afford (P)-3-chloro-4-((3,5-difluoropyridin-2-yl)methoxy-d)-5′,6-dimethyl-2-oxo-2H-[1,4′-bipyridine]-2′-carboxylate (S)-1-(naphthalen-1-yl)ethan-1-aminium (Isomer I-salt) (CPD-23 Salt A) (17 g, 93.4%) with (Isomer I:Isomer II) 98.48%: 1.52% (97.16% ee) chiral purity by chiral HPLC. (P)-3-chloro-4-((3,5-difluoropyridin-2-yl)methoxy-d)-5′,6-dimethyl-2-oxo-2H-[1,4′-bipyridine]-2′-carboxylate (S)-1-(naphthalen-1-yl)ethan-1-aminium (Isomer I-salt) (CPD-23 Salt A) (17 g, 28.62 mmol, 1.0 eq) was de-salted by the procedure mentioned above to afford (P)-3-chloro-4-((3,5-difluoropyridin-2-yl)methoxy-d)-5′,6-dimethyl-2-oxo-2H-[1,4′-bipyridine]-2′-carboxylate (Isomer I) (CPD-23) and (M)-3-chloro-4-((3,5-difluoropyridin-2-yl)methoxy-d)-5′,6-dim ethyl-2-oxo-2H-[1,4′-bipyridine]-2′-carboxylate (Isomer II) (CPD-30) as an off-white solid 11.5 g (yield=95) with 99.64% purity by HPLC, (Isomer I:Isomer II) 99.92%:0.08% (99.84 ee), and 96.65% D1-purity by LCMS. ¹H-NMR (400 MHz, DMSO-d₆) δ ppm: 13.36 (1H, s), 8.80 (1H, s), 8.60 (1H, s), 8.12-8.06 (1H, m), 7.97 (1H, s), 6.79 (1H, s), 5.45 (1H, s), 2.07 (3H, s), 1.92 (3H, s). MS(ES) m/z 423.26 (M+H).

Step 5: Preparation of (P)-chloro-4-((3,5-difluoropyridin-2-yl)methoxy-d)-N-methoxy-N,5′,6-trimethyl-2-oxo-2H-[1,4′-bipyridine]-2′-carboxamide (CPD-24).

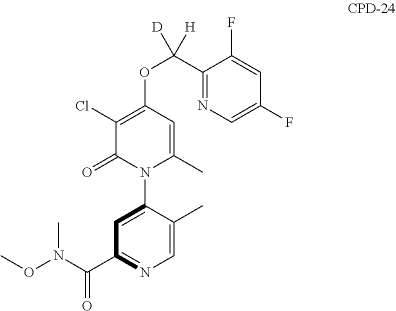

To a stirred solution of (P)-3-chloro-4-((3,5-difluoropyridin-2-yl)methoxy-d)-5′,6-dimethyl-2-oxo-2H-[1,4′-bipyridine]-2′-carboxylate (Isomer I) (CPD-23) (Example 10, Step 4, 12.0 g, 28.38 mmol, 1.0 eq., (Isomer I:Isomer II) 99.92%: 0.08% (99.84% ee)) in DCM (96 ml, 8.0 vol.) at 0-5° C., EDC.HCl (5.98 g, 31.221 mmol, 1.1 eq.) was added to the reaction mass under argon atmosphere and stirred for 15 min. Then added N, O-dimethyl hydroxylamine hydrochloride (4.15 g, 42.574 mmol, 1.5 eq.) at 0-5° C. and stirred for 15 min. TEA (5.08 ml, 35.478 mmol, 1.25 eq.) was added drop-wise to the reaction mass and stirred for 2 h. Progress of the reaction was monitored by TLC and HPLC. After reaction completion the reaction was quenched with water (120 ml, 10 vol.). It was allowed to 25-35° C. and Stirred for 10 mins. The two layers were separated and aqueous layer was extracted with DCM (2×60 ml, 5 vol.). Combined DCM layer was washed with water (2×36 ml), dried and concentrated under vacuum. The crude was co-distilled with MTBE (3×24 ml) completely. Then fourth time crude solid was suspended in MTBE (24 ml, 2 vol.) and stirred for 2 h. Filtered the solid, washed with MTBE (12 ml, 1 vol.) and dried under vacuum to afford (P)-chloro-4-((3,5-difluoropyridin-2-yl)methoxy-d)-N-methoxy-N,5′,6-trimethyl-2-oxo-2H-[1,4′-bipyridine]-2′-carboxamide (CPD-24) as an off-white solid 11.0 g (yield=83.2%) with 99.62% purity by HPLC, (Isomer I:Isomer II) 99.29%:0.12% (99.17% ee) chiral purity chiral HPLC, 96.95% D1-purity by LCMS. ¹H-NMR (400 MHz, DMSO-d₆) δ ppm: 8.70 (1H, s), 8.60 (1H, d, J=2.4 Hz), 8.11-8.06 (1H, m), 7.62 (1H, s), 6.79 (1H, s), 5.45 (1H, s), 3.67 (3H, s), 3.31 (3H, s), 2.04 (3H, s), 1.93 (3H, s). MS(ES) m/z 466.22 (M+H).

Step 6: Preparation of (P)-2′-acetyl-3-chloro-4 #3,5-difluoropyridin-2-yl)methoxy-d)-5′,6-dimethyl-2H-[1,4′-bipyridin]-2-one (CPD-25).

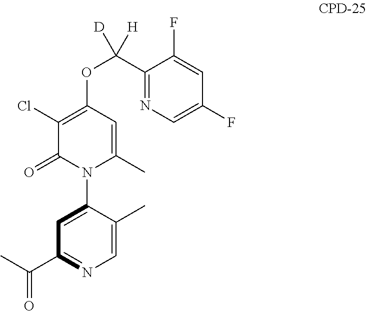

A stirred solution of (P)-chloro-4-((3,5-difluoropyridin-2-yl)methoxy-d)-N-methoxy-N,5′,6-trimethyl-2-oxo-2H-[1,4′-bipyridine]-2′-carboxamide (CPD-24) (Example 10, Step 5, 9 g, 19.317 mmol, 1.0 eq, (Isomer I:Isomer II) 99.29%:0.12% (99.17% ee)) in THF (90 ml, 10 vol.) was cooled to −10° C. Methyl magnesium bromide solution 2M in THF (14.4 ml, 1.5 eq.) was added slowly drop wise to the reaction mass under argon atmosphere and stirred at −10° C. to 0° C. for 2 h. The progress of the reaction was monitored by TLC and HPLC. After completion of reaction the reaction was quenched with saturated NH₄C1 (180 ml, 20 vol), allowed to −25° C. (— 1 h). It was extracted with EtOAc (90 ml×3). Combined EtOAc layer was washed with water (2×45 ml) and brine solution. EtOAc layer were completely distilled and the residue was co-distilled with MeOH (2×18 ml). Charged MeOH (45 ml, 5 vol.) and stirred for 1 h at 60° C. and allow to rt. Solid was filtered, washed with MeOH (9 ml, 1 vol). It was and dried under vacuum below 40° C., to afford (P)-2′-acetyl-3-chloro-4-((3,5-difluoropyridin-2-yl)methoxy-d)-5′,6-dimethyl-2H-[1,4′-bipyridin]-2-one (CPD-25) as a pale yellow solid 6.5 g (yield=80%) with 97.72% purity by HPLC, (Isomer I:Isomer II) 99.81%:0.19% (99.62% ee) chiral purity by chiral HPLC, 96.78% D1-purity by LCMS. ¹H-NMR (400 MHz, DMSO-d₆) δ ppm: 8.83 (1H, s), 8.60 (1H, d, J=2.4 Hz), 8.12-8.06 (1H, m), 7.89 (1H, s), 6.79 (1H, s), 5.45 (1H, d, J=2.8 Hz), 2.66 (3H, s), 2.09 (3H, s), 1.91 (3H, s). MS(ES) m/z 421.16 (M+H).

Step 7: Preparation of (P)-(E)-3-chloro-4-((3,5-difluoropyridin-2-yl)methoxy-d)-2′-(3-(dimethylamino)acryloyl)-5′,6-dimethyl-2H-[1,4′-bipyridin]-2-one (CPD-26)

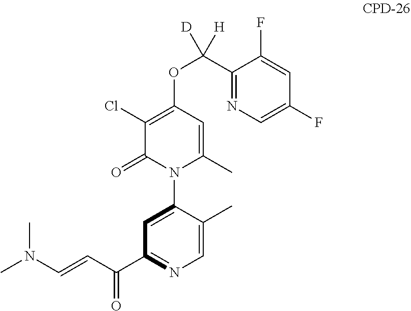

To a stirred suspension of (P)-2′-acetyl-3-chloro-4-((3,5-difluoropyridin-2-yl)methoxy-d)-5′,6-dimethyl-2H-[1,4′-bipyridin]-2-one (CPD-25) (Example 10, Step 6, 4.5 g, 10.71 mmol, 1.0 eq, (Isomer I:Isomer II) 99.81%:0.19% (99.62% ee)) in DMF (4.5 ml, 1 vol) was added DMF-DMA (3.0 eq) at 25-30° C. Then reaction mass was heated to 50-55° C. and maintained for 16 h (Note—throughout the reaction mass as a suspension). Reaction progress was monitored by TLC/HPLC. After reaction completion the reaction mixture was cooled to RT and diluted with ethyl acetate (10 ml) stirred for 1 h. Solid was filtered and washed with EtOAc (5 ml). It was suck dried well and then dried under vacuum at 40° C. for 1 h, afford (P)-(E)-3-chloro-4-((3,5-difluoropyridin-2-yl)methoxy-d)-2′-(3-(dimethylamino)-acryloyl)-5′,6-dimethyl-2H-[1,4′-bipyridin]-2-one (CPD-26) as pale yellow solid 4.1 g (yield=80.5%) with 99.00% purity by HPLC, (Isomer I:Isomer II) 99.75%:0.25% (99.5% ee) chiral purity by chiral HPLC, 96.68% D1-purity by LCMS. ¹H-NMR (400 MHz, DMSO-d₆) δ ppm: 8.71 (1H, s), 8.60 (1H, d, J=2.4 Hz), 8.11-8.06 (1H, m), 7.85 (1H, s), 7.82 (1H, d, J=3.6 Hz), 6.78 (1H, s), 6.38 (1H, d, J=12.4 Hz), 5.45 (1H, s), 3.19 (3H, s), 2.94 (3H, s), 2.05 (3H, s), 1.91 (3H, s). MS(ES) m/z 476.24 (M+H).

Step 8: Preparation of (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II)

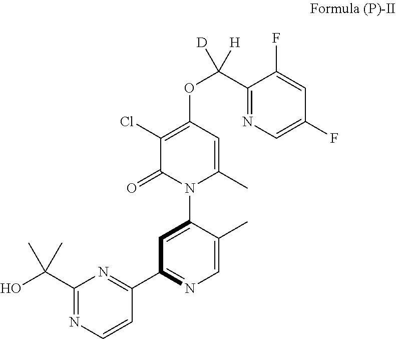

To a stirred solution of (P)-(E)-3-chloro-4-((3,5-difluoropyridin-2-yl)methoxy-d)-2′-(3-(dimethylamino)acryloyl)-5′,6-dimethyl-2H-[1,4′-bipyridin]-2-one (CPD-26) (Example 10, Step 7, 6.0 g, 12.61 mmol, 1.0 eq, (Isomer I:Isomer II) 99.75%:0.25% (99.5% ee)) in DMF (36 ml, 6 vol), potassium carbonate (4.35 g, 31.52 mmol, 2.5 eq) was added portion wise at RT. Reaction mass was stirred for 30 min, 1-amino-2-hydroxy-2-methylpropan-1-iminium (INT-02) (5.2 g, 37.83 mmol, 3.0 eq) was added portion wise at RT. The reaction mass was heated at 45-50° C. (inner temp) for 18 h. Homogenous solution was observed after 18 h. Reaction progress was monitored by HPLC & TLC. After reaction completion the reaction mixture was cooled to 10-15° C. and stirred for 30 min. Ice-cold water (120 ml) was added to get solid. Further stirred for 1 h at 10-15° C. Solid was filtered, washed with water (60 ml, 10 vol) and dried under vacuum. It was co-distilled with MeOH (2×30 mml, 5 vol) and dried. The solid (6.7 g), was taken in rbf, methanol (36 ml, 6 vol) was added and heated at 55-60° C. to afford clear solution. It was cooled to 10-15° C. and stirred for 2 h. It was filtered and washed with methanol (6 ml), dried under vacuum to afford (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II) (3.2 g, 49.3%). Filtrates was filtered again to afford (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II) as an off-white solid 1.7 g (yield=26.19%) with 98.37% purity by HPLC, (Isomer I:Isomer II) 99.05%:0.95% (98.1% ee), and 96.82% D1-purity by LCMS. ¹H-NMR (400 MHz), DMSO-d6 δ ppm: 8.97 (1H, d, J=5.2 Hz), 8.86 (1H, s), 8.69 (1H, s), 8.61 (1H, d, J=2.4 Hz), 8.24 (1H, d, J=5.2 Hz), 8.12-8.07 (1H, m), 6.84 (1H, s), 5.47 (1H, s), 5.24 (1H, s), 2.10 (3H, s), 1.98 (3H, s), 1.54 (6H, d, J=4.8 Hz). MS(ES) m/z 515.29 (M+H).

Step 9: Preparation (crystallization) of (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II)

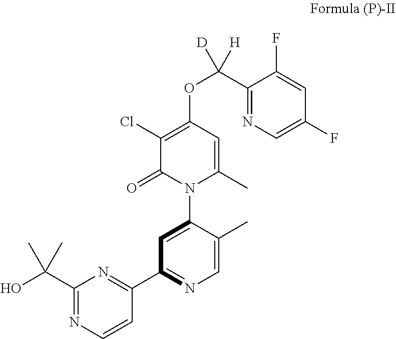

A suspension of (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II) (Example 10, Step 8, 3.2 g, 5.43 mmol, (Isomer I:Isomer II) 99.05%:0.95% (98.1% ee)) in isopropyl alcohol (64 ml, 20 vol) was heated to 65-70° C. and stirred for 1 h (clear solution was observed). Reaction mass was filtered hot at 65-70° C. Filtrate allowed to RT and stirred for 16 h. Reaction was monitored by TLC/HPLC. After completion of the reaction the reaction mass was filtered and the solid and washed with isopropyl alcohol (6.4 ml, 2 vol), The solid was dried under vacuum at 40° C. to afford (P)-3-chloro-4-((3,5-difluoropyridin-2-yl)methoxy-d)-2′-(2-(2-hydroxypropan-2-yl)pyrimidin-4-yl)-5′,6-dimethyl-2H-[1,4′-bipyridin]-2-one (Formula (P)-II) as an off-white solid 2.35 g (yield=73.4%) with 98.99% purity by HPLC, (Isomer I:Isomer II) 99.99%:0.01% (99.98 ee) chiral purity by chiral HPLC, and 96.57% D1-purity by LCMS. ¹H-NMR (400 MHz), DMSO-d6 δ ppm: 8.97 (1H, d, J=5.2 Hz), 8.86 (1H, s), 8.68 (1H, s), 8.61 (1H, d, J=2.4 Hz), 8.24 (1H, d, J=5.2 Hz), 8.12-8.07 (1H, m), 6.84 (1H, s), 5.47 (1H, s), 5.24 (1H, s), 2.10 (3H, s), 1.98 (3H, s), 1.53 (3H, s), 1.52 (3H, s). MS(ES) m/z 515.29 (M+H).

### Example 11: Preparation of (P)-2′-chloro-4-((3,5-difluoropyridin-2-yl)methoxy-d2)-5′,6-dimethyl-2H-[1,4′-bipyridin]-2-one (Formula (P)-I) on Kilogram Scale

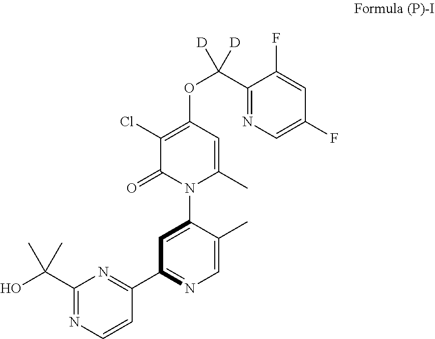

Step 1: Preparation of 2′-chloro-4-((3,5-difluoropyridin-2-yl)methoxy-d2)-5′,6-dimethyl-2H-[1,4′-bipyridin]-2-one (CPD-03)

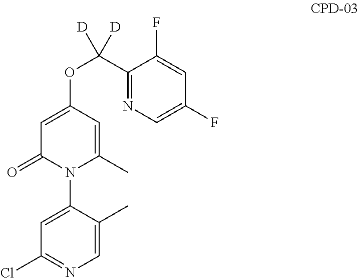 [SMILES: [2H]C([2H])(Oc1cc(C)n(-c2cc(Cl)ncc2C)c(=O)c1)c1ncc(F)cc1F]

To a stirred suspension of 2′-chloro-4-hydroxy-5′,6-dimethyl-2H-[1,4′-bipyridin]-2-one (CPD-02) (20.0 kg, 79.78 mol, 1.0 eq.) in DMF (5 vol), was added K₂CO₃ (16.52 kg, 119.5 mol, 1.5 eq.) at 25-30° C. and stirred for 30 minutes. Then added a solution of 2-(chloromethyl-d2)-3,5-difluoropyridine (INT-01) (16.46 kg, 99.68 mol, 1.25 eq.) in DMF (1 vol.) at 25-35° C. The reaction mixture was stirred for another 40-50 hours. The progress of the reaction was monitored by TLC/HPLC. After completion of the reaction charged MTBE (80.0 L, 4.0 vol.) into reaction mass and stirred for 30 minutes. Reaction mass was added to pre cooed (5-10° C.) water (400 L, 20 vol) at 5-10° C. and stirred for 6-8 hours at 5-10° C. The separated solid was filtered and washed with water (60.0 L, 3.0 vol.), suck dried the solid until the complete expulsion of MLs′. Unloaded the solid, charged wet solid into the reactor followed by water (200 L, 10 vol.) and stirred for 1-2 hours. Filtered the solid and washed the solid with water (60.0 L, 3.0 vol.). Dried the solid under vacuum at below 50° C. to afford 2′-chloro-4-((3,5-difluoropyridin-2-yl)methoxy-d2)-5′,6-dimethyl-2H-[1,4′-bipyridin]-2-one (CPD-03) as an off-white solid 23.20 kg (yield=76.82%) with 88.50% purity by HPLC. ¹H-NMR (500 MHz, DMSO-d₆) δ ppm: 8.59-8.58 (1H, d, J=2.4 Hz), 8.49-8.48 (1H, d, J=4.8 Hz), 8.09-8.04 (1H, m), 7.60 (1H, s), 6.13-6.12 (1H, d, J=2.0 Hz), 6.03-6.02 (1H, d, J=2.8 Hz), 1.98 (3H, s), 1.85 (3H, s). MS(ES) m/z 380.13 (M+H).

Step 2: Preparation of 2′,3-dichloro-4-((3,5-difluoropyridin-2-yl)methoxy-d2)-5′,6-dimethyl-2H-[1,4′-bipyridin]-2-one (CPD-08)

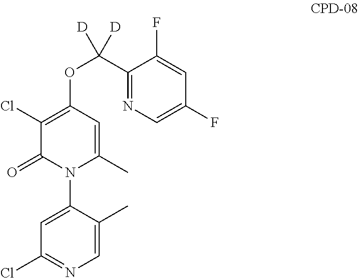 [SMILES: [2H]C([2H])(Oc1cc(C)n(-c2cc(Cl)ncc2C)c(=O)c1Cl)c1ncc(F)cc1F]

To a stirred solution of 2′-chloro-4-((3,5-difluoropyridin-2-yl)methoxy-d2)-5′,6-dimethyl-2H-[1,4′-bipyridin]-2-one (CPD-03) (Example 11, Step 1, 23.0 kg, 60.55 mol, 1.0 eq.) in IPA (345 L, 15 vol.), was added Dichloroacetic acid (1.95 kg, 15.12 mol, 0.25 eq.) and heated to 60-65° C. and N-chlorosuccinimide (9.70 kg, 72.64 mol, 1.2 eq.) was added at 60-65° C. The reaction was stirred at 65-70° C. and maintained for 2-4 hrs. After the completion of reaction, the reaction mass was cooled to 25-35° C. over a period of 2-3 hours. Filtered the solid and washed the solid with IPA (69.0 L, 3.0 vol.). Dried the solid under vacuum at below 50° C. to afford 2′,3-dichloro-4-((3,5-difluoropyridin-2-yl)methoxy-d2)-5′,6-dimethyl-2H-[1,4′-bipyridin]-2-one (CPD-08) as an off-white solid 9.70 kg (yield=76.82%) with 94.50% HPLC purity. ¹H-NMR (400 MHz, DMSO-d₆) δ ppm: 8.60-8.59 (1H, d, J=2 Hz), 8.53 (1H, s), 8.11-8.06 (1H, m), 7.68 (1H, s), 6.798-6.796 (1H, s), 1.97 (3H, s), 1.95 (3H, s). MS(ES) m/z 414.12 (M+H).

Step 3: Preparation of 3-chloro-4-((3,5-difluoropyridin-2-yl)methoxy-d2)-5′,6-dimethyl-2-oxo-2H-[1,4′-bipyridine]-2′-carboxylic acid (CPD-09)

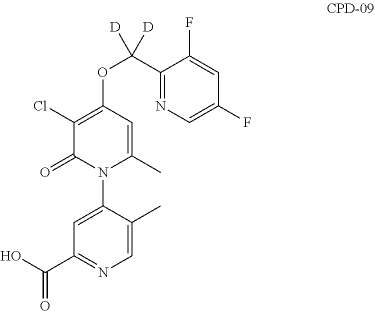 [SMILES: [2H]C([2H])(Oc1cc(C)n(-c2cc(C(=O)O)ncc2C)c(=O)c1Cl)c1ncc(F)cc1F]

To a stirred solution of 2′,3-dichloro-4-((3,5-difluoropyridin-2-yl)methoxy-d2)-5′,6-dimethyl-2H-[1,4′-bipyridin]-2-one (CPD-08) (Example 11, Step 2, 6.5 kg, 15.69 mol, 1.0 eq.), in acetonitrile (52.0 L, 8.0 vol.) and water (26.0 L, 4.0 vol.) was added Li₂CO₃ (3.47 kg, 46.96 mol, 3.0 eq.) Purged the reaction mass with argon gas for 1-2 hours. Charged Pd(dppf)Cl₂ (0.062 kg, 0.077 mol, 0.005 eq.) into the reaction mass and purged the reaction mass with argon gas for 30 minutes. Then applied CO pressure 15-20 psi and released CO pressure. Applied CO pressure 100 psi and slowly reaction mass heated to 75-80° C. and maintained for 18-20 hours. The progress of the reaction was monitored by HPLC. After completion of the reaction, reaction mass was cooled to 25-35° C. and de-gassed with argon. The reaction mass was added to the water (130 L, 20.0 vol.). pH of the reaction mass was adjusted to ˜13.0 with 2N NaOH solution (52.0 L, 8.0 vol.). Filtered the reaction mass through hyflo bed and washed with water (13.0 L, 2.0 vol.). Filtered mLs' were extracted with MTBE (32.5 L, 3×, 5.0 vol.). The pH of the aqueous layer was adjusted to ˜2.0 with 6N HCl (32.5 L, 5.0 vol.) at 10-15° C. and stirred the reaction mass for 6-8 hours at 25-35° C. The separated solid was filtered and washed with water (32.5 L, 5.0 vol.) followed by IPA (6.5 L, 1.0 vol.). Dried the solid under vacuum at below 50° C. to afford 3-chloro-4-((3,5-difluoropyridin-2-yl)methoxy-d2)-5′,6-dimethyl-2-oxo-2H-[1,4′-bipyridine]-2′-carboxylic acid (CPD-09) as an off-white solid 5.76 g (yield=86.87%) with 98.40% purity by HPLC. ¹H-NMR (400 MHz, DMSO-d₆) δ ppm: 13.36 (1H, s), 8.80 (1H, s), 8.60-8.59 (1H, s), 8.12-8.06 (1H, m), 7.97 (1H, s), 6.799-6.797 (1H, s), 2.08 (3H, s), 1.93 (3H, s). MS(ES) m/z 424.12 (M+H).

Step 4: Preparation of (P)-3-chloro-4-((3,5-difluoropyridin-2-yl)methoxy-d2)-5′,6-dimethyl-2-oxo-2H-[1,4′-bipyridine]-2′-carboxylate (S)-1-(naphthalen-1-yl)ethan-1-aminium (Isomer I-salt) (CPD-10 Salt A) and (M)-3-chloro-4-((3,5-difluoropyridin-2-yl)methoxy-d2)-5′,6-dimethyl-2-oxo-2H-[1,4′-bipyridine]-2′-carboxylate (S)-1-(naphthalen-1-yl)ethan-1-aminium (Isomer II-salt) (CPD-28 Salt A)

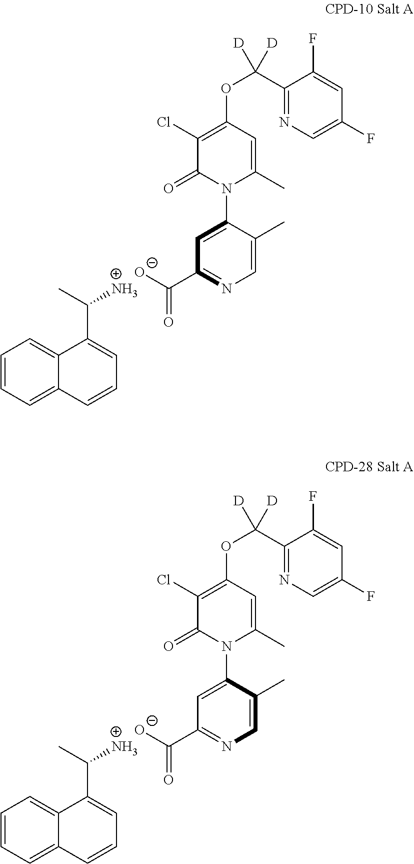

To a stirred solution of 2′-acetyl-3-chloro-4-((3,5-difluoropyridin-2-yl)methoxy-d2)-5′,6-dimethyl-2H-[1,4′-bipyridin]-2-one (CPD-09) (Example 11, Step 3, 6.80 kg, 16.04 mol, 1.0 eq.) in 5% of DMSO in anisole (47.6 L, 7.0 vol.) was slowly heated to 110-115° C. and stirred for 10-15 min. A solution of (S)-2-naphthylethyl amine (2.88 kg, 16.81 mol, 1.05 eq.) in anisole (95.2 L, 14.0 vol.) was slowly added to the reaction mass over a period of 20-24 hours. The progress of the reaction was monitored by Chiral HPLC. After completion of reaction, reaction mass was cooled to 25-35° C. and maintained for 2-4 hours. The resulting reaction mass was filtered and washed with ethanol (68.0 L, 10 vol.). Dried the solid under vacuum at below 50° C. to afford (P)-3-chloro-4-((3,5-difluoropyridin-2-yl)methoxy-d2)-5′,6-dimethyl-2-oxo-2H-[1,4′-bipyridine]-2′-carboxylate (S)-1-(naphthalen-1-yl)ethan-1-aminium (Isomer I-salt) (CPD-10 Salt A) and (M)-3-chloro-4-((3,5-difluoropyridin-2-yl)methoxy-d2)-5′,6-dimethyl-2-oxo-2H-[1,4′-bipyridine]-2′-carboxylate (S)-1-(naphthalen-1-yl)ethan-1-aminium (Isomer II-salt) (CPD-28 Salt A) total of both (S)-2-naphthylethyl amine salts 8.30 kg (yield=87.18%) with 98.35% purity by HPLC, (Isomer I:Isomer II) 99.89%:0.11% (99.78% ee) chiral purity by chiral HPLC. MS(ES) m/z 424.14 (M+H).

Step 5: Preparation of (P)-3-chloro-4-((3,5-difluoropyridin-2-yl)methoxy-d2)-5′,6-dimethyl-2-oxo-2H-[1,4′-bipyridine]-2′-carboxylic acid (CPD-10)

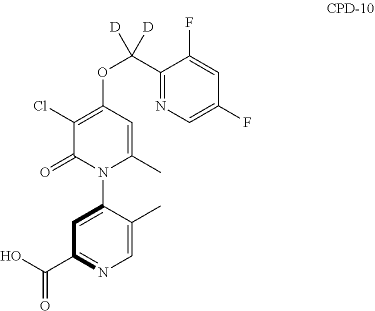

The mixture of salts (P)-3-chloro-4-((3,5-difluoropyridin-2-yl)methoxy-d2)-5′,6-dimethyl-2-oxo-2H-[1,4′-bipyridine]-2′-carboxylate (S)-1-(naphthalen-1-yl)ethan-1-aminium (Isomer I-salt) (CPD-10 Salt A) and (M)-3-chloro-4-((3,5-difluoropyridin-2-yl)methoxy-d2)-5′,6-dimethyl-2-oxo-2H-[1,4′-bipyridine]-2′-carboxylate (S)-1-(naphthalen-1-yl)ethan-1-aminium (Isomer II-salt) (CPD-28 Salt A) (Example 11, Step 4, 8.30 kg, 13.94 mol, 1.0 eq., (Isomer I:Isomer II) 99.89%: 0.11% (99.78% ee) was dissolved in water (166 L, 20 vol.) and was cooled to 10-15° C. The pH of the reaction mass was adjusted to ˜13.0 with 2N NaOH solution (66.4 L, 8.0 vol.). Filtered the reaction mass through hyflo bed and washed with purified water (16.6 L, 2.0 vol.). Filtered mLs were extracted with MTBE (41.5 L, 3×5.0 vol.). The pH of the aqueous layer was adjusted to ˜2.0 with 6N HCl (41.5 L, 5.0 vol.) at 10-15° C. The reaction mass was stirred for 6-8 hours at 25-35° C. Filtered the solid, washed the solid with water (41.5 L, 5.0 vol.) followed by IPA (8.30 L, 1.0 vol.). Dried the solid under vacuum at below 50° C. to afford (P)-3-chloro-4-((3,5-difluoropyridin-2-yl)methoxy-d2)-5′,6-dimethyl-2-oxo-2H-[1,4′-bipyridine]-2′-carboxylic acid (CPD-10) as off-white solid 5.37 kg (yield=78.90%) with 98.35 HPLC purity, (Isomer I:Isomer II) 99.89%:0.11% (99.78% ee) chiral purity by chiral HPLC. 1H-NMR (400 MHz, DMSO-d₆) δ ppm: 13.35 (1H, s), 8.80 (1H, s), 8.60-8.59 (1H, d, J=2.4 Hz), 8.12-8.06 (1H, m), 7.97 (1H, s), 6.798-6.796 (1H, d, J=0.8 Hz), 2.08 (3H, s), 1.929-1.928 (3H, s). MS(ES) m/z 424.16 (M+H).

Step 6: Preparation of (P)-3-chloro-4-((3,5-difluoropyridin-2-yl)methoxy-d2)-N-methoxy-N,5′,6-trimethyl-2-oxo-2H-[1,4′-bipyridine]-2′-carboxamide (CPD-11)

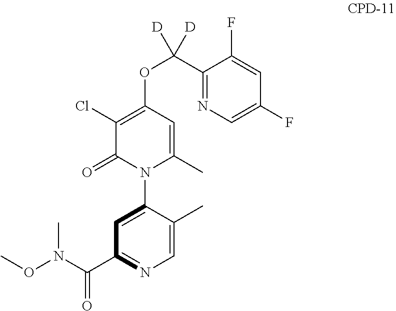

To a stirred solution of (P)-3-chloro-4-((3,5-difluoropyridin-2-yl)methoxy-d2)-5′,6-dimethyl-2-oxo-2H-[1,4′-bipyridine]-2′-carboxylic acid (CPD-10) (Example 11, Step 5, 1.25 kg, 2.94 mol, 1.0 eq.), (Isomer I:Isomer II) 99.89%: 0.11% (99.78 ee) in DCM (9.6 L, 8.0 vol was added EDC.HCl (0.621 kg, 3.23 mol, 1.1 eq.) followed by N, O-dimethyl hydroxylamine hydrochloride (0.431 kg, 4.41 mol, 1.5 eq.) at −5 to 0° C. under argon atmosphere. The resulting reaction mass was stirred for 5-10 minutes. TEA (0.372 kg, 3.67 mol, 1.25 eq.) was added dropwise to the reaction mass and stirred for 2-4 hours at −5 to 0° C. under argon atmosphere. The progress of the reaction was monitored by HPLC. After completion of reaction, reaction mass was quenched with water (12.5 L, 10 vol.) at below 10° C. Allowed the reaction mass to 25-35° C. Stirred for 10 minutes and separated the DCM layer. Aqueous layer was extracted with DCM (6.25 L, 2×5 vol.). Combined DCM layers were washed with water (6.25 L, 5.0 vol.) followed by 10% aq. NaCl solution (6.25 L, 5.0 vol.). Organic layer was dried over sodium sulphate (1.25 kg, 1.0 T) and washed with DCM (2.5 L, 2.0 vol.). Distilled the DCM layer up to 2-3 vol. under vacuum at below 40° C. and co-distilled with MTBE (3.75 L, 2×3.0 vol.) up to 1-2 vol. Then charged MTBE (5.0 L, 4.0 vol.) and stirred for 4-6 hours. Filtered the solid and washed the solid with MTBE (2.5 L, 2.0 vol.). Dried the solid under vacuum at below 50° C. to afford (P)-3-chloro-4-((3,5-difluoropyridin-2-yl)methoxy-d2)-N-methoxy-N,5′,6-trimethyl-2-oxo-2H-[1,4′-bipyridine]-2′-carboxamide (CPD-11) as an off white solid 1.25 kg (yield=91.20%) with 96.29 purity by HPLC, (Isomer I:Isomer II) 99.81%: 0.19% (99.62% ee) chiral purity by chiral HPLC. ¹H-NMR (400 MHz, DMSO-d₆) δ ppm: 8.70 (1H, s), 8.60-8.59 (1H, d, J=2.4 Hz), 8.11-8.06 (1H, m), 7.63 (1H, s), 6.797-6.795 (1H, d, J=0.8 Hz), 3.68 (3H, s), 3.32-3.29 (3H, d, J=11.2 Hz), 2.05 (3H, s), 1.93 (3H, s). MS(ES) m/z 467.18 (M+H).

Step 7: Preparation of (P)-2′-acetyl-3-chloro-4-((3,5-difluoropyridin-2-yl)methoxy-d2)-5′,6-dimethyl-2H-[1,4′-bipyridin]-2-one (CPD-12)

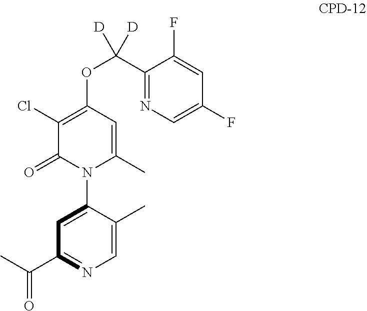

A stirred suspension of (P)-3-chloro-4-((3,5-difluoropyridin-2-yl)methoxy-d2)-N-methoxy-N,5′,6-trimethyl-2-oxo-2H-[1,4′-bipyridine]-2′-carboxamide (CPD-11) (Example 11, Step 6, 1.2 kg, 2.57 mol, 1.0 eq.), (Isomer I:Isomer II) 99.81%:0.19% (99.62% ee) in THF (12.0 L, 10 vol.) was cooled to −10° C. to 0° C. under argon atmosphere. Methyl magnesium chloride solution (3M in THF, 1.028 L, 3.08 mol, 1.2 eq.) was added to the reaction mass under argon atmosphere and stirred at −10° C. to 0° C. for 2 h. The progress of the reaction was monitored by HPLC. After completion of the reaction, reaction mass was quenched with 10% NH₄C1 (7.2 L, 6.0 vol.) at below 5.0° C. and stirred for 10-20 minutes. Distil the reaction mass under vacuum at below 50° C. up to 5-6 vol. Charged water (4.8 L, 4.0 vol.) to the distilled crude and stirred for 4-6 hours at 25-35° C. Filtered the solid and washed the solid with water (6.0 L, 5.0 vol.) followed by IPA (1.2 L, 1.0 vol.). Dried the solid under vacuum at below 50° C. to afford (P)-2′-acetyl-3-chloro-4-((3,5-difluoropyridin-2-yl)methoxy-d2)-5′,6-dimethyl-2H-[1,4′-bipyridin]-2-one (CPD-12) as an off white solid, 0.95 kg (yield=87.96%) with 95.76% purity by HPLC, (Isomer I:Isomer II) 99.85%: 0.15% (99.70% ee) chiral purity by chiral HPLC. ¹H-NMR (400 MHz, DMSO-d₆) δ ppm: 8.83 (1H, s), 8.60-8.59 (1H, d, J=2.4 Hz), 8.12-8.06 (1H, m), 7.89 (1H, s), 6.793-6.792 (1H, d, J=0.4 Hz), 2.66 (3H, s), 2.09 (3H, s), 1.91 (3H, s). MS(ES) m/z 422.18 (M+H).

Step 8: Preparation of (P)-(E)-3-chloro-4-((3,5-difluoropyridin-2-yl)methoxy-d2)-2′-(3-(dimethylamino)acryloyl)-5′,6-dimethyl-2H-[1,4′-bipyridin]-2-one (CPD-13)

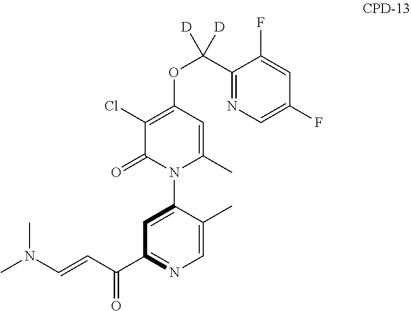

To a stirred suspension of (P)-2′-acetyl-3-chloro-4-((3,5-difluoropyridin-2-yl)methoxy-d2)-5′,6-dimethyl-2H-[1,4′-bipyridin]-2-one (CPD-12) (Example 11, Step 7, 1.8 kg, 4.26 mol, 1.0 eq., (Isomer I:Isomer II) 99.85%:0.15% (99.70% ee) in DMF (1.8 L, 1.0 vol.) was added DMF-DMA (1.77 kg, 14.85 mol, 3.5 eq.) at 25-30° C. Then reaction mass was heated to 60-65° C. and maintained for 18-20 hours. The progress of the reaction was monitored by HPLC. After completion of the reaction, reaction mass was cooled to 25-35° C. Charged THF (0.9 L, 0.5 vol.) and stirred for 1-2 minutes. Filtered the solid and washed the solid with THF (1.8 L, 1.0 vol.). Dried the solid under vacuum at below 50° C. to afford (P)-(E)-3-chloro-4-((3,5-difluoropyridin-2-yl)methoxy-d2)-2′-(3-(dimethylamino)acryloyl)-5′,6-dimethyl-2H-[1,4′-bipyridin]-2-one (CPD-13) as a pale yellow solid 1.60 kg (yield=78.81%) with 94.60% purity by HPLC, (Isomer I:Isomer II) 99.90%: 0.10% (99.80% ee) chiral purity by chiral HPLC. ¹H-NMR (400 MHz), DMSO-d₆δ ppm: 8.71 (1H, s), 8.60-8.59 (1H, d, J=2.4 Hz), 8.11-8.06 (1H, m), 7.85 (1H, s), 7.82-7.81 (1H, d, J=2.4 Hz), 6.784-6.782 (1H, d, J=0.8 Hz) 6.38-6.35 (1H, d, 12.4 Hz), 3.19 (3H, s), 2.94 (3H, s), 2.05 (3H, s), 1.914-1.913 (3H, d, J=0.4 Hz). MS(ES) m/z 477.18 (M+H).

Step 9: Preparation of (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I)

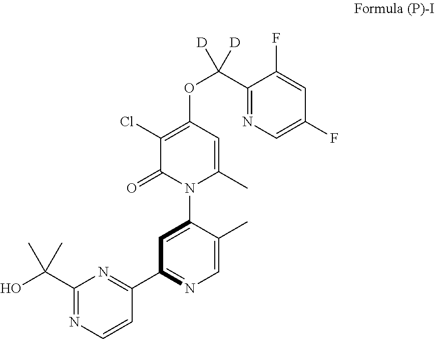

To a stirred solution of (P)-(E)-3-chloro-4-((3,5-difluoropyridin-2-yl)methoxy-d2)-2′-(3-(dimethylamino)acryloyl)-5′,6-dimethyl-2H-[1,4′-bipyridin]-2-one (CPD-13) (Example 11, Step 8, 0.8 kg, 1.67 mol, 1.0 eq.), (Isomer I:Isomer II) 99.90%:0.10% (99.80% ee) in DMF (4.8 L, 6.0 vol.) potassium carbonate (0.579 kg, 4.18 mol, 2.5 eq) was added portion wise at RT. Reaction mass was stirred for 30 min, 1-amino-2-hydroxy-2-methylpropan-1-iminium (INT-02) (0.697 kg, 5.02 mol, 3.0 eq.) at 25-35° C. under argon purging. The reaction mass was slowly heated to 45-50° C. and maintained for 12-14 hours at 45-50° C. under argon purging. The progress of the reaction was monitored by HPLC. After completion of the reaction, reaction mass was cooled to 5-10° C. Slowly added water (16.0 L 20 vol.) to the reaction mass at 5-10° C. and maintained for 4-6 hours. Filtered the solid and washed the solid with water (8.0 L, 10 vol.), suck dried the solid until the complete expulsion of mLs′. Charged the wet solid into RBF followed by DCM (8.0 L, 10 vol.) and stirred for 30 minutes. Charged water (8.0 L, 10 vol.) to the reaction mass and stirred for 10-20 minutes. Separated both the layers and DCM layer was washed with water (4.0 L 5.0 vol.) followed by 10% aqueous NaCl solution (4.0 L 5.0 vol.). Dried the organic layer over sodium sulphate (0.8 kg, 1.0 T) and washed with DCM (1.6 L, 2.0 vol.). Charged DCM layer into RBF followed by activated carbon (0.08 kg, 0.1 T). The reaction mass was heated to 35-40° C. and maintained for 1 hours. Cooled the reaction mass to 25-35° C. and filtered the reaction mass through hyflo bed, washed with DCM (4.0 L, 5.0 vol.). Distilled the filtrate under vacuum at below 40° C. up to 1-2 vol. and co-distilled with IPA (2.4 L, 2×3.0 vol.) up to 1-2 vol. Charged IPA (12.0 L, 15 vol.) to the reaction mass and heated to 70-75° C. Stirred the reaction mass for 1-2 hours at 70-75° C. The reaction mass was cooled to 25-35° C. and maintained for 2-4 hours. Reaction mass was further cooled to 5-10° C. and stirred for 2-4 hours. Filtered the solid and washed the solid with IPA (1.6 L, 2.0 vol.). Dried the solid under vacuum at below 50° C. to afford (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I) as an off white solid 0.665 kg (yield=76.96%) with 99.40% purity by HPLC, (Isomer I:Isomer II) 99.91%: 0.09% (99.82% ee) purity by chiral HPLC. ¹H-NMR (400 MHz), DMSO-d₆ δ ppm: 8.97 (1H, d, J=5.2 Hz), 8.86 (1H, s), 8.69 (1H, s), 8.61 (1H, d, J=2.4 Hz), 8.24 (1H, d, J=5.2 Hz), 8.12-8.07 (1H, m), 6.84 (1H, s), 5.24 (1H, s), 2.10 (3H, s), 1.98 (3H, s), 1.53 (6H, s). MS(ES) m/z 516.19 (M+H).

### Example 12: Preparation of (P)-2′-chloro-4-((3,5-difluoropyridin-2-yl)methoxy-d2)-5′,6-dimethyl-2H-[1,4′-bipyridin]-2-one (Formula (P)-I) Telescoped Reaction with Crystallization

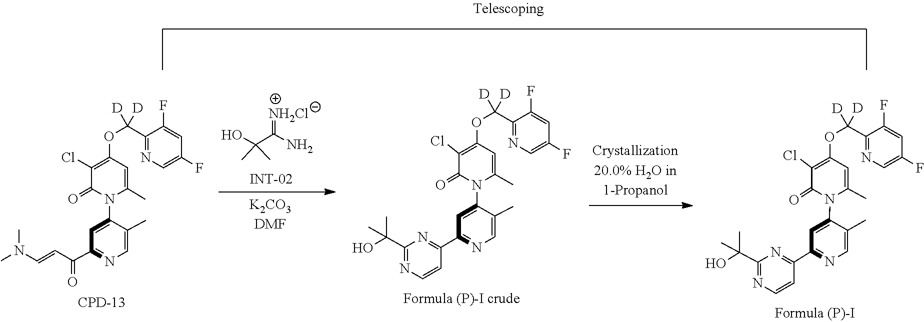

To a stirred solution of (P)-(E)-3-chloro-4-((3,5-difluoropyridin-2-yl)methoxy-d2)-2′-(3-(dimethylamino)acryloyl)-5′,6-dimethyl-2H-[1,4′-bipyridin]-2-one (CPD-13, Example 7, Step 8) (100.0 g, 1.0 eq., HPLC Purity 99.12%, Isomer 1:Isomer 2, 100.0%:0.0) in DMF (0.698 L, 6.0 vol.) at 25-35° C. was added was added K₂CO₃ (72.4 g, 2.5 eq.) under argon purging. After stirring for 5-10 min at 25-35° C., 2-hydroxy-2-methylpropionamidine HCl (INT-02) (87.2 g, 3.0 eq.) at 25-35° C. was added under argon purging. The reaction mass was slowly warmed to 45-50° C. and was stirred at that temperature for 12-14 h. Progress of the reaction was monitored by TLC/HPLC. After the reaction was completed, it was cooled to 10-15° C., diluted with water (2.0 L, 20.0 vol.), and stirred for 2-4 h at 10-15° C. The solid was filtered, washed with water (1.0 L, 10.0 vol.) and sucked dry the solid until the complete expulsion of mother liquors. The wet solid was charged to the reactor and DCM (1.0 L, 10.0 vol.) was added. The reaction mass stirred for 30 mins followed by the addition of water (0.5 L, 5.0 vol.) and the reaction mass continued to stir for another 10-20 mins. Organic layer was washed with water (0.5 L, 5.0 vol.) followed by 10% aqueous sodium chloride solution (0.5 L, 5.0 vol.). The organic layer was dried over sodium sulfate (100 g, 0.1 T) and washed with DCM (0.20 L, 2.0 vol.). Charge the ENO-PC carbon (10.0 g, 0.1 T) into reactor and raise the temperature to 35-40° C. and maintain for 1 h. Cool the reaction mass to 25-35° C. and filter the reaction mass through hyflo bed and wash the hyflo bed with DCM (0.5 L, 5.0 vol.). Distil the filtrate completely under vacuum at below 40° C. to afford 89.9 g (yield 83.1%) crude (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I) with HPLC purity 96.87% and HPLC chiral purity (Isomer 1:Isomer 2, 99.97%:0.03). Charged crude (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I) (DCM solids) and 20% H₂O in 1-Propanol (55.0 mL, 5.5 vol.) into RBF at 25-35° C. Raise the reaction mass temperature to 53-56° C. and stir for 10-15 min. Filter the reaction mass and wash with 20% H₂O in 1-Propanol (10.0 mL, 1.0 vol.) Stir the reaction mass for 10-15 min. at 53-56° C. Cool the reaction mass to 43-46° C. Charge seed material (0.3 g, 0.03 T) into the reaction mass at 43-46° C. Stir the reaction mass for 1 h at 43-46° C. Slowly add H₂O (77.0 mL, 7.7 vol.) into the reaction mass over a period of 10 h at 43-46° C. Cool the reaction mass to 20-22° C. and stir for 30 h. Filter the solid and wash with 20% H₂O in 1-Propanol (10.0 mL, 1.0 vol.) and dried to afford 66.8 g (yield: 74.3%) of (P)-3-chloro-4-((3,5-difluoropyridin-2-yl)methoxy-d2)-2′-(2-(2-hydroxypropan-2-yl)pyrimidin-4-yl)-5′,6-dimethyl-2H-[1,4′-bipyridin]-2-one (Formula (P)-I) with HPLC purity 99.94% and HPLC chiral purity (Isomer 1:Isomer 2, 99.98%:0.02%). 1H-NMR (400 MHz, DMSO-d₆): δ ppm 8.97 (1H, d, J=5.2 Hz), 8.85 (1H, s), 8.68 (1H, s), 8.61 (1H, d, J=2.4 Hz), 8.24 (1H, d, J=5.2 Hz), 8.12-8.07 (1H, m), 6.84 (1H, s), 5.24 (1H, s), 2.10 (3H, s), 1.98 (3H, s), 1.53 (3H, s), 1.52 (3H, s); MS(ES) m/z 516.26 (M+H).

### Example 13: Preparation of (P)-2′-chloro-4-((3,5-difluoropyridin-2-yl)methoxy)-5′,6-dimethyl-2H-[1,4′-bipyridin]-2-one (Formula (P)-A) Telescoped Reaction with Crystallization

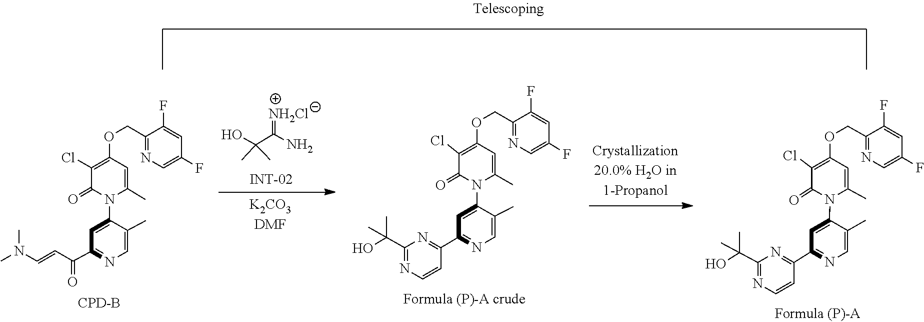

To a stirred solution of (P)-(E)-3-Chloro-4-((3,5-difluoropyridin-2-yl)methoxy)-2′-(3-(dimethyl-amino)acryloyl)-5′,6-dimethyl-2H-[1,4′-bipyridin]-2-one (Example 10 Step 3 or Example 12 Step 3 of US2022/0235025A1) (180.0 g, 1.0 eq., Isomer 1:Isomer 2, 99.96%:0.04%) in DMF (1.08 L, 6.0 vol.) at 25-35° C. was added was added K₂CO₃ (130.9 g, 2.5 eq.) under argon purging. After stirring for 5-10 min at 25-35° C., 2-hydroxy-2-methylpropionamidine HCl (INT-02) (157.5 g, 3.0 eq.) at 25-35° C. was added under argon purging. The reaction mass was slowly warmed to 45-50° C. and was stirred at that temperature for 12-14 h. Progress of the reaction was monitored by TLC/HPLC. After the reaction was completed, it was cooled to 10-15° C., diluted with water (3.60 L, 20.0 vol.), and stirred for 2-4 h at 10-15° C. The solid was filtered, washed with water (1.80 L, 10.0 vol.) and sucked dry the solid until the complete expulsion of mother liquors. The wet solid was charged to the reactor and DCM (1.80 L, 10.0 vol.) was added. The reaction mass stirred for 30 mins followed by the addition of water (0.9 L, 5.0 vol.) and the reaction mass continued to stir for another 10-20 mins. Organic layer was washed with water (0.9 L, 5.0 vol.) followed by 10% aqueous sodium chloride solution (0.9 L, 5.0 vol.). The organic layer was dried over sodium sulfate (180 g, 0.1 T) and washed with DCM (0.36 L, 2.0 vol.). Charge the ENO-PC carbon (18.0 g, 0.1 T) into reactor and raise the temperature to 35-40° C. and maintain for 1 h. Cool the reaction mass to 25-35° C. and filter the reaction mass through hyflo bed and wash the hyflo bed with DCM (0.9 L, 5.0 vol.). Distil the filtrate completely under vacuum at below 40° C. to afford 160.0 g (yield 82.3%) crude (P)-3-Chloro-4-((3,5-difluoropyridin-2-yl)methoxy)-2′-(2-(2-hydroxypropan-2-yl)-pyrimidin-4-yl)-5′,6-dimethyl-2H-[1,4′-bipyridin]-2-one (Formula (P)-A) with HPLC purity 96.07% and HPLC chiral purity (Isomer 1:Isomer 2, 99.79%:0.21%). Charged crude (P)-3-Chloro-4-((3,5-difluoropyridin-2-yl)methoxy)-2′-(2-(2-hydroxypropan-2-yl)-pyrimidin-4-yl)-5′,6-dimethyl-2H-[1,4′-bipyridin]-2-one (Formula (P)-A) with HPLC purity 96.07% and HPLC chiral purity (Isomer 1:Isomer 2, 99.79%:0.21%) (DCM solid) (10 g, 1.0 eq.) and 20% H₂O in 1-Propanol (55.0 mL, 5.5 vol.) into RBF at 25-35° C. Raise the reaction mass temperature to 53-56° C. and stir for 10-15 min. Filter the reaction mass and wash with 20% H₂O in 1-Propanol (10.0 mL, 1.0 vol.) Stir the reaction mass for 10-15 min. at 53-56° C. Cool the reaction mass to 43-46° C. Charge seed material (0.3 g, 0.03 T) into the reaction mass at 43-46° C. Stir the reaction mass for 1 h at 43-46° C. Slowly add H₂O (77.0 mL, 7.7 vol.) into the reaction mass over a period of 10 h at 43-46° C. Cool the reaction mass to 20-22° C. and stir for 30 h. Filter the solid and wash with 20% H₂O in 1-Propanol (10.0 mL, 1.0 vol.) and dried to afford 7.31 g (yield: 73.1%) of (P)-3-Chloro-4-((3,5-difluoropyridin-2-yl)methoxy)-2′-(2-(2-hydroxypropan-2-yl)-pyrimidin-4-yl)-5′,6-dimethyl-2H-[1,4′-bipyridin]-2-one (Formula (P)-A) with HPLC purity 99.92% and HPLC chiral purity (Isomer 1:Isomer 2, 99.94%:0.06%). ¹H-NMR (400 MHz, DMSO-d₆): δ ppm 8.97 (d, 1H, J=5.2 Hz), 8.86 (s, 1H), 8.69 (s, 1H), 8.61 (d, 1H, J=2.4 Hz), 8.24 (d, 1H, J=5.2 Hz), 8.06-8.14 (m, 1H), 6.84 (s, 1H), 5.49 (d, 2H, J=1.2 Hz), 5.25 (s, 1H), 2.10 (s, 3H), 1.98 (s, 3H), 1.04 (s, 3H), 1.03 (s, 3H); MS (ES) m/z 514.37 (M+H).

1. A process for the preparation of compound of Formula (P)-I having the structure: 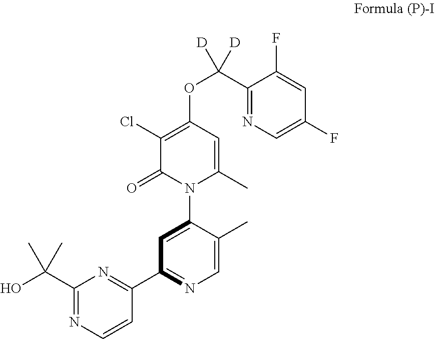
comprising the steps of:
(a) contacting the compound 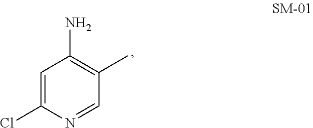
with the compound 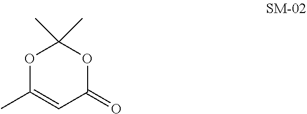
in the presence of dimethylacetemide (DMAc) to form a mixture; and
(b) contacting the mixture of (a) with an alcoholic HCl solution
to form the compound 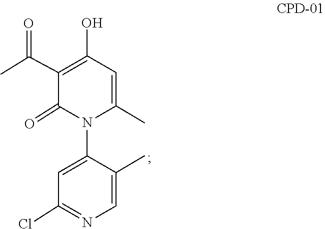 [SMILES: CC(=O)c1c(O)cc(C)n(-c2cc(Cl)ncc2C)c1=O]
and
(c) converting CPD-01 to Formula (P)-I.

2. (canceled)

3. The process according to claim 1, further comprising contacting the compound CPD-01 with H₂₅₀₄ to form the compound

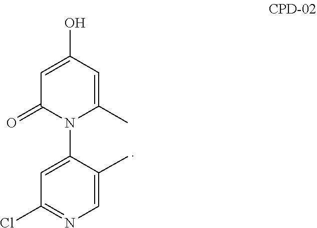 [SMILES: Cc1cnc(Cl)cc1-n1c(C)cc(O)cc1=O]

4. The process according to claim 3, further comprising contacting the compound CPD-02 with the compound 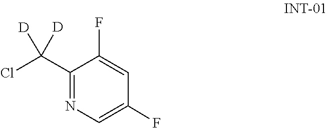 [SMILES: [2H]C([2H])(Cl)c1ncc(F)cc1F]
and a base to form the compound

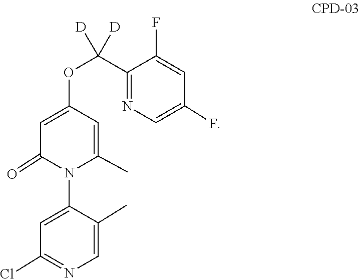 [SMILES: [2H]C([2H])(Oc1cc(C)n(-c2cc(Cl)ncc2C)c(=O)c1)c1ncc(F)cc1F]

5.-29. (canceled)

30. The process according to claim 4, further comprising contacting the compound CPD-03 with a chlorination reagent to form the compound

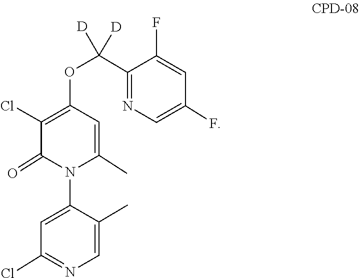 [SMILES: [2H]C([2H])(Oc1cc(C)n(-c2cc(Cl)ncc2C)c(=O)c1Cl)c1ncc(F)cc1F]

31.-32. (canceled)

33. The process according to claim 0, further comprising contacting CPD-08 with CO in the presence of a palladium catalyst, a base, and a solvent mixture to form the compound

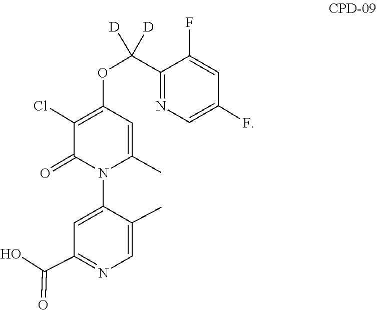 [SMILES: [2H]C([2H])(Oc1cc(C)n(-c2cc(C(=O)O)ncc2C)c(=O)c1Cl)c1ncc(F)cc1F]

34.-40. (canceled)

41. The process according to claim 0, further comprising subjecting the compound CPD-09 to chiral separation with a chiral amine and a solvent to obtain the compound

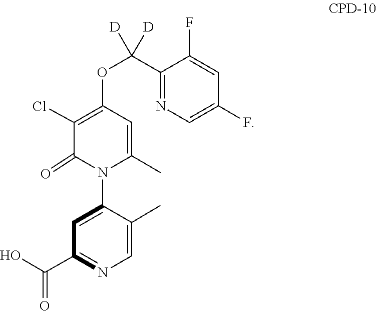

42. The process according to claim 0, wherein the chiral amine is selected from the group consisting of (5)-1-(naphthalen-2-yl)ethan-1-amine and (1S, 2R)-2-amino-1,2-diphenylethan-1-ol.

43. The process according to claim 0, wherein the solvent is selected from the group consisting of toluene, ethylbenzene, n-butanol, anisole, DMSO, or a combination thereof.

44. The process according to claim 0, further comprising contacting the compound CPD-10 with a solvent, MeNHOMe, an amine base, and a coupling reagent to obtain the compound

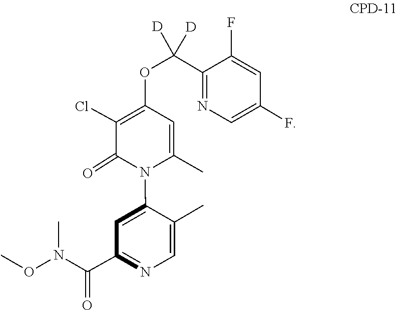

45.-47. (canceled)

48. The process according to claim 44, further comprising contacting the compound CPD-11 with MeMgX to obtain the compound

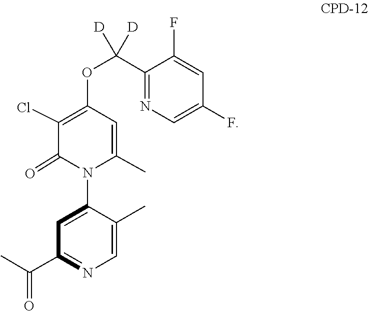

49. (canceled)

50. The process according to claim 0, further comprising condensing compound CPD-12 with N,N-dimethyl-formamide dimethyl acetal to obtain the compound

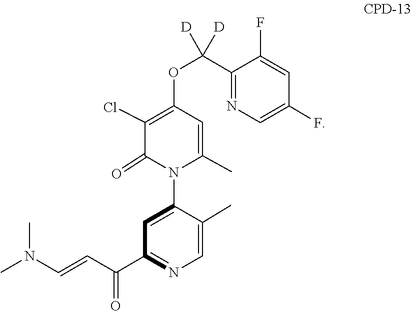

51. The process according to claim 50, further comprising contacting the compound CPD-13 with 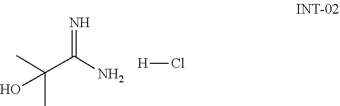
in the presence of a base, and forming the compound of Formula (P)-I.

52. The process according to claim 51, wherein the base is selected from the group consisting of K₂CO₃, N,N-diisopropylethylamine (DIPEA), triethylamine (TEA), tBuOK, tBuONa, and Cs₂CO₃.

53.-101. (canceled)

102. The process of claim 50, further comprising contacting the compound CPD-13 with 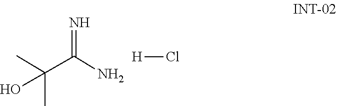
in the presence of a base, forming the crude compound of Formula (P)-I, and crystallizing the crude compound of Formula (P)-I with a crystallization solvent mixture to yield a crystallized compound of Formula (P)-I.

103. The process according to claim 102, wherein the base is selected from the group consisting of K₂CO₃, N,N-diisopropylethylamine (DIPEA), triethylamine (TEA), tBuOK, tBuONa, and Cs₂CO₃.

104. The process according to claim 102, wherein the crystallization solvent mixture is water and 1-propanol.

105.-107. (canceled)

108. A process for the preparation of compound of Formula (P)-A having the structure: 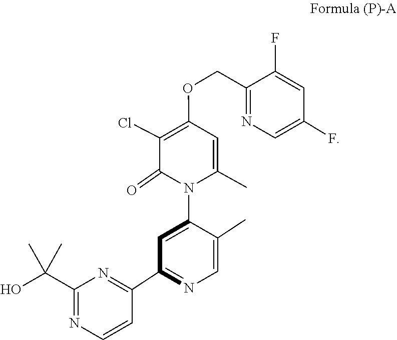
comprising:
contacting the compound 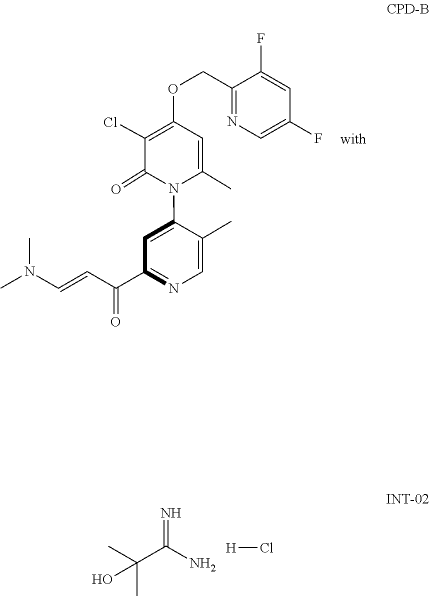
in the presence of a base, forming the crude compound of Formula (P)-A, and crystallizing the crude compound of Formula (P)-A with a crystallization solvent mixture to yield a crystallized compound of Formula (P)-A.

109. The process according to claim 108, wherein the base is selected from the group consisting of K₂CO₃, N,N-diisopropylethylamine (DIPEA), triethylamine (TEA), tBuOK, tBuONa, and Cs₂CO₃.

110. The process according to claim 108, wherein the crystallization solvent mixture is water and 1-propanol.

111.-116. (canceled)

117. A compound, or a salt thereof, or a co-crystal thereof, selected from the group consisting of: 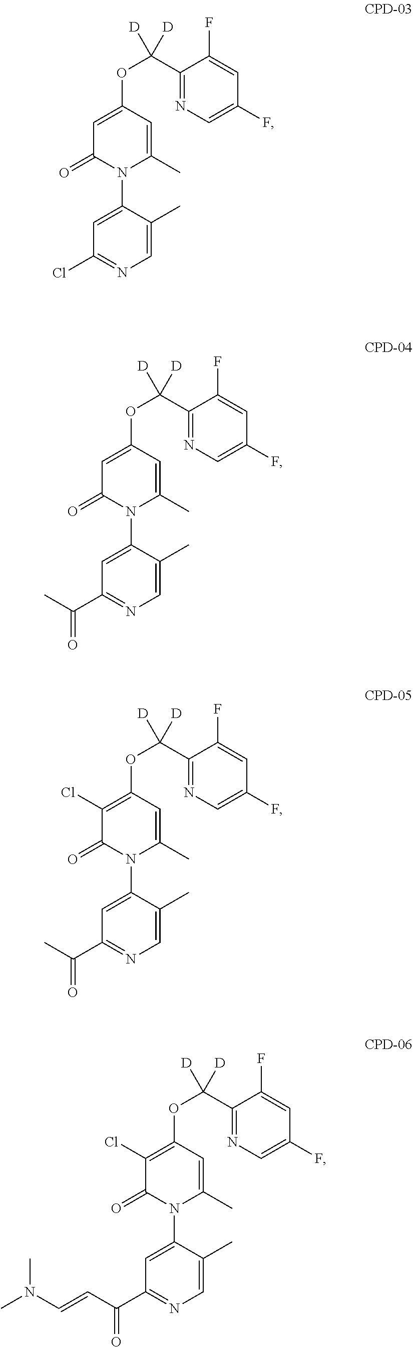 [SMILES: [2H]C([2H])(Oc1cc(C)n(-c2cc(Cl)ncc2C)c(=O)c1)c1ncc(F)cc1F ; [2H]C([2H])(Oc1cc(C)n(-c2cc(C(C)=O)ncc2C)c(=O)c1)c1ncc(F)cc1F ; [2H]C([2H])(Oc1cc(C)n(-c2cc(C(C)=O)ncc2C)c(=O)c1Cl)c1ncc(F)cc1F ; [2H]C([2H])(Oc1cc(C)n(-c2cc(C(=O)/C=C/N(C)C)ncc2C)c(=O)c1Cl)c1ncc(F)cc1F] 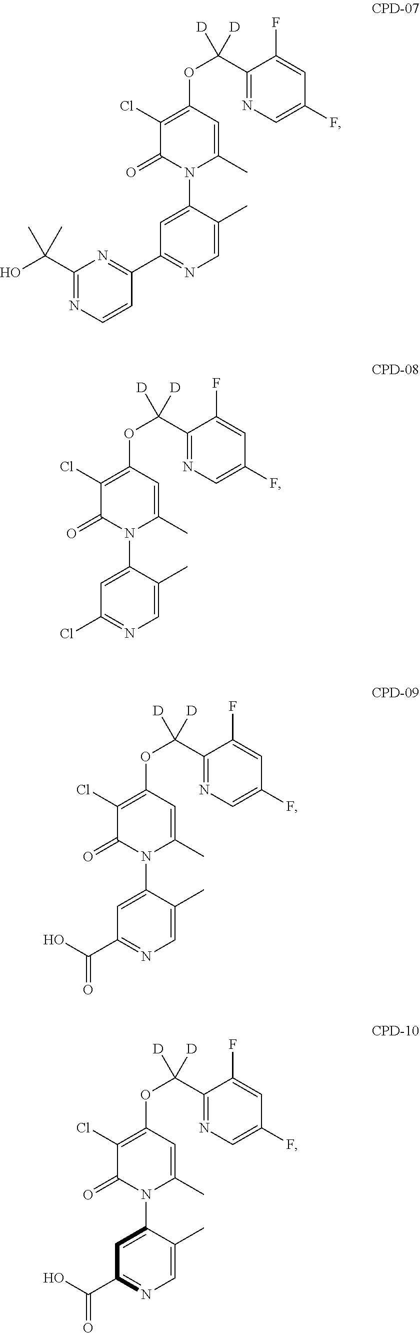 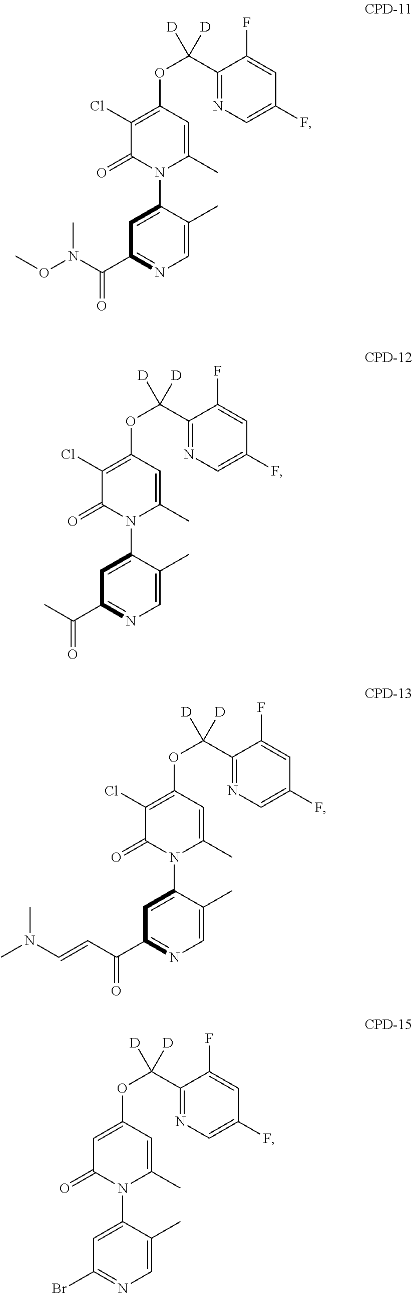 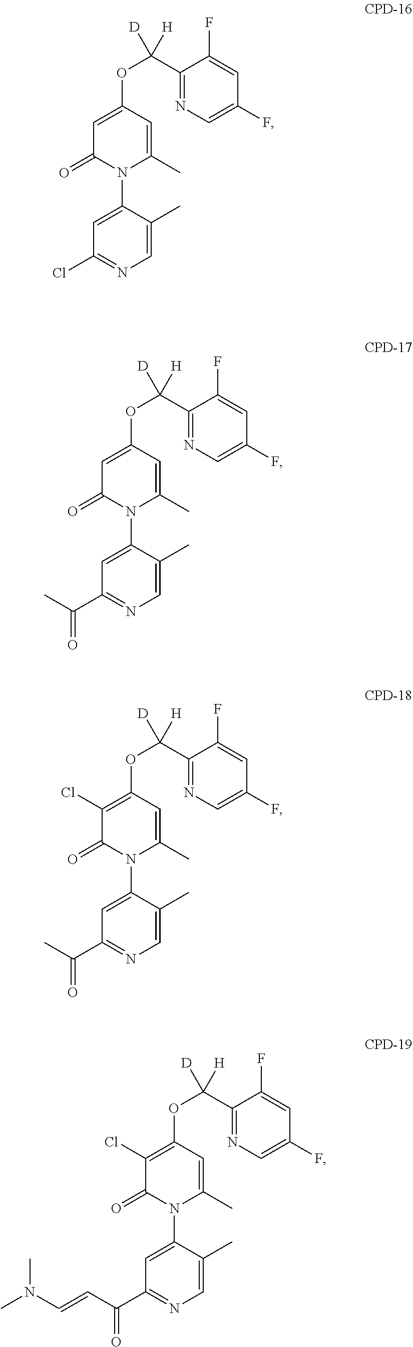 [SMILES: [2H]C(Oc1cc(C)n(-c2cc(Cl)ncc2C)c(=O)c1)c1ncc(F)cc1F ; [2H]C(Oc1cc(C)n(-c2cc(C(C)=O)ncc2C)c(=O)c1)c1ncc(F)cc1F ; [2H]C(Oc1cc(C)n(-c2cc(C(C)=O)ncc2C)c(=O)c1Cl)c1ncc(F)cc1F ; [2H]C(Oc1cc(C)n(-c2cc(C(=O)/C=C/N(C)C)ncc2C)c(=O)c1Cl)c1ncc(F)cc1F] 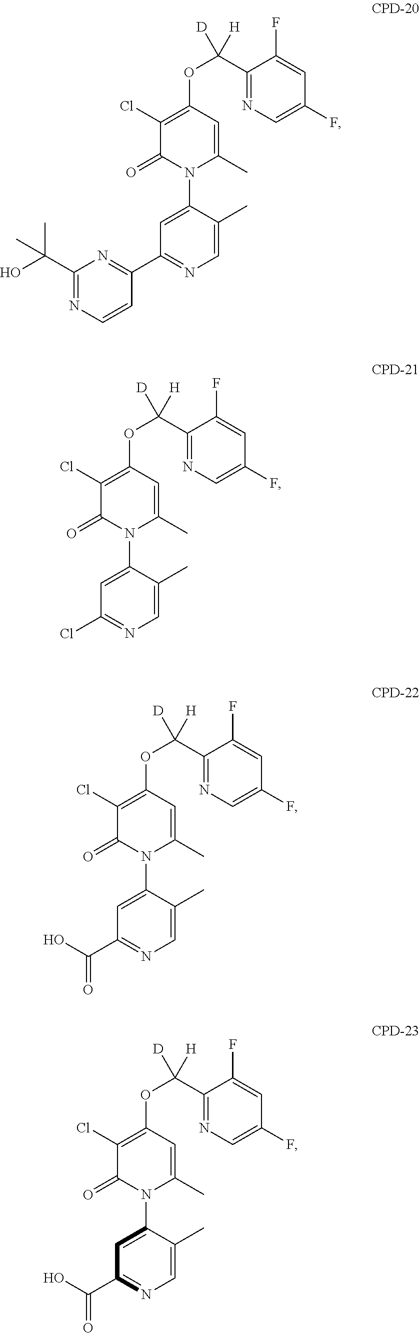 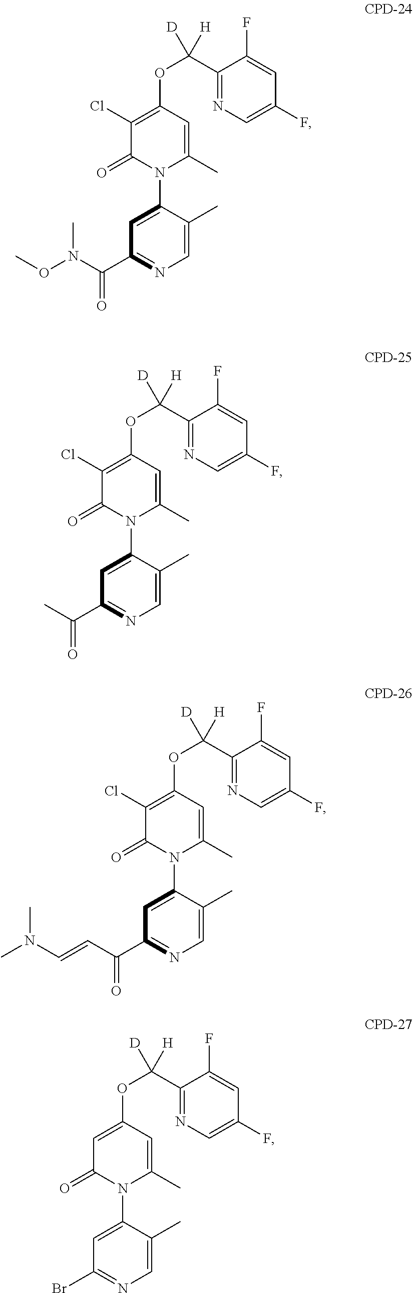 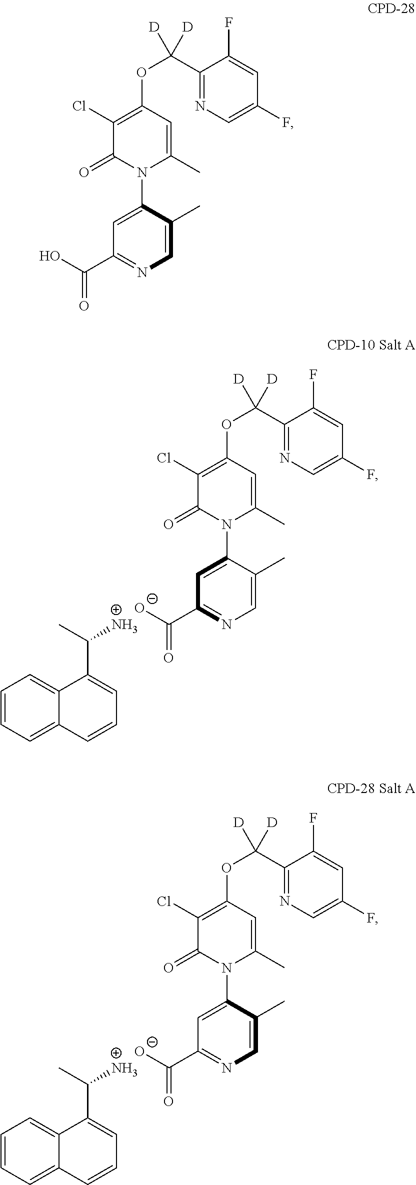 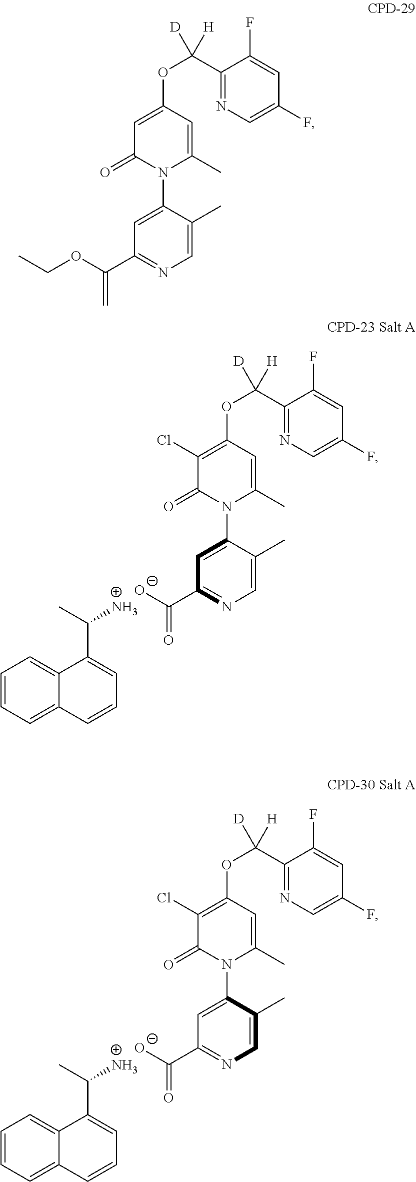 [chemical structure]
